(19) United States
(12) Patent Application Publication
(10) Pub. No.: US 2025/0090698 A1
(43) Pub. Date: Mar. 20, 2025

(54) SMALL MOLECULE INHIBITORS FOR EARLY DIAGNOSIS OF PROSTATE SPECIFIC MEMBRANE ANTIGEN CANCERS AND NEURODEGENERATIVE DISEASES

(71) Applicant: INDIAN INSTITUTE OF TECHNOLOGY INDORE, Indore (IN)

(72) Inventors: Venkatesh Chelvam, Indore (IN); Sagnik Sengupta, Indore (IN); Mena Asha Krishnan, Indore (IN); Amit Pandit, Indore (IN)

(21) Appl. No.: 18/943,338

(22) Filed: Nov. 11, 2024

Related U.S. Application Data
(63) Continuation-in-part of application No. 16/695,851, filed on Nov. 26, 2019, now abandoned.

(30) Foreign Application Priority Data
Nov. 27, 2018 (IN) 201821044594

(51) Int. Cl. A61K 51/04; C07B 59/00

(52) U.S. Cl. CPC A61K 51/0497; A61K 51/0402; C07B 59/001; A61K 2121/00; A61K 2123/00; C07B 2200/05

(57) ABSTRACT

Accordingly, embodiments herein disclose a conjugate D-E-F having binding affinity≤60 nM; wherein D comprises AAPT ligand or derivative thereof, E comprises a spacer comprising AA1, AA2, AA3 and/or AA4 and F is a chelating group. The conjugate also comprising AAPT ligand conjugated arene chelating linker for delivery of ^{99m}Tc radioisotope. Another embodiment also discloses a AAPT-PCa DOTA bioconjugate. An embodiment also discloses method of solid phase synthesis of AAPT-ligand. It also discloses a method of solid phase synthesis of AAPT-PCa DOTA bioconjugate for delivering of radioisotopes.

### CROSS-REFERENCE TO RELATED APPLICATIONS

The present application is a Continuation-in-part (CIP) of U.S. application Ser. No. 16/695,851 and claims priority from an Indian Application Number 201821044594 filed on Nov. 27, 2018, the disclosure of which is hereby incorporated by reference herein

### FIELD OF INVENTION

Biologically active molecules that are conjugated to ligands capable of binding to prostate specific membrane antigen (PSMA) via a linker may be useful for diagnosis, imaging and treatment of related diseases that involve pathogenic cell populations over-expressing PSMA. The present disclosure described herein pertains to compounds and methods for diagnosis and treatment of malignancy arising out of prostate, brain, breast, bladder tissues and few of the neurodegenerative diseases like schizophrenia and ALS.

The embodiments of the present disclosure described herein pertain to the synthesis of several biologically active conjugates which comprises of a novel PSMA inhibitor or ligand that binds with nano-molar affinity to PSMA over-expressed on malignant or pathological cells, a peptidic spacer with a strategic handle for introduction of NIR fluorescent tag for intra-operative imaging and a linker with hydrophobic spacer for interaction with the third hydrophobic pocket of PSMA which is further bonded with a chelating core for tagging radioisotopes for radionuclear imaging and treatment of cancers, especially in prostate, brain, breast, bladder and few of the neurodegenerative diseases.

The present disclosure relates to small molecule inhibitors or ligands for diagnosis and treatment of cancers such as prostate, brain, breast, etc., and other neurodegenerative diseases.

### BACKGROUND OF INVENTION

In recent years, there has been a surge in the development of both diagnostics and therapeutics that take advantage of the expression and activity of PSMA/GCPII. Prostate cancer (PCa) and breast cancer ranks first in terms of cancer-related deaths among men and women in the United States whereas it is lung followed by breast in Indian population in 2017 with prostate ranking third among all cancers in Indian men accounting for 7% of all cases. As per the American Cancer Society report in 2017, about 1,688,780 new cancer cases are expected to be diagnosed with cancer becoming the leading cause of mortality surpassing cardiovascular disease worldwide. It is estimated that 22 million people would be diagnosed for cancer by 2030 resulting in 13 million deaths every year.

PSMA or GCPII's exact function in cancer is unknown. However, many studies have linked its role to tumour progression and carcinogenesis. In the brain, PSMA or GCPII metabolizes the neurotransmitter NAAG and it has now been identified as a target for therapeutic interventions and diagnostics in various neurological disorders and also in cancers of prostate, breast, bladder, brain etc.

Early diagnosis of prostate cancer is most commonly achieved by blood test for prostate specific antigen (PSA), or a prostate biopsy. Later stages of prostate cancer can be detected by performing a biopsy which is extremely painful and expensive. The PSA diagnostic test has been widely criticized due to its inaccuracy in diseases like benign prostatic hypertrophy (BPH) and prostatitis. Axumin is a recently approved radioactive diagnostic agent by the Food and Drug Administration (FDA) for prostate carcinoma. It is generally used during positron emission tomography (PET) imaging in men based on elevated prostate specific antigen (PSA) levels who are suspected with recurring cases of prostate cancer after undergoing treatment.

Another FDA approved diagnostic agent for prostate cancer, marketed as ProstaScint, is an ¹¹¹In-labeled anti-PSMA monoclonal antibody [¹¹¹In]7E11-C5 imaging agent for detection of metastasis in patients with prostate cancer. However, long circulating half-life of monoclonal antibodies in plasma along with their low permeability in tumors limit the success of these antibodies in diagnosis of this kind of cancer. It has been discovered that biologically active compounds that are conjugated to inhibitors or ligands capable of binding to prostate specific membrane antigen (PSMA) via a linker may be useful for radio as well as optical imaging in diagnosis and treatment of cancers. Few small molecule inhibitors for PSMA are well documented in the literature with nanomolar binding affinity; 2-[3-(1,3-dicarboxypropyl)ureido]pentanedioic acid (DUPA), (S)-2-(3-((S)-5-amino-1-carboxypentyl)ureido) pentanedioic acid, which are now in various stages of clinical trials. This area therefore requires further research and development.

In existing diagnostic methods like digital rectal examination (DRE), blood test for prostate specific antigen (PSA) and biopsy are inconclusive, can give false positive results, and are highly inaccurate and insensitive for detection of prostate cancer. Although prostate enlargement and growth asymmetry can be examined by transrectal ultrasound jointly with magnetic resonance imaging (MRI) and computerized tomography (CT) these diagnostic tests are costly and difficult to afford on routine basis. The primary cause of PCa is the emergence of castration-resistant prostate cancer (CRPC) and subsequent metastasis followed by chemoresistance for which there is no known cure. Early stage prostate tumors can be treated by surgery, radiation, and androgen-deprivation therapy (ADT). However, recurrence takes place within 2-3 years and the mean survival time for CRPC is only 16-18 months.

During the last few decades there have been observed increase in the development of a variety of imaging techniques such as positron emission tomography scanning, magnetic resonance imaging and computed tomography. These techniques have greatly improved and facilitated the detection of cancers. However, the most important factor which facilitates the survival of patients suffering from cancer is the complete surgical resection of the tumor. The complete surgical removal of the tumor prevents chances of tumor recurrence and the most important aspect required for this is the differentiation between tumor and normal tissue during surgery. Visual inspection and evaluation of the tumor margins are now typically assessed intraoperatively. However, it is the possibility of a microinvasion of the cancer to surrounding tissues which increases the difficulty in determining an excision margin that is completely tumor free. This difficulty finally forces surgeons to perform wide excisions during surgery which lead to the damage of healthy tissues resulting in loss of functional structures. Once surgeons are able to visually evaluate the minimum safety margins during tumor excision clearly, it will be possible to completely remove the tumor. Therefore, there is a critical, unmet need for accurate cost effective diagnostic tool that can detect and treat early stages of prostate cancer or other related neuro degenerative diseases involving PSMA protein. In future, NIR fluorescent small molecule conjugates would be used as an intra-operative diagnostic tool for detection and surgical of PSMA expressing malignant masses during cancer surgery.

Moreover, in western countries few small molecule PSMA radiopharmaceuticals have reached human clinical trials in addition to three approved diagnostic agents (ProstaScint, Choline C11 and Axumin) by FDA for detection of PCa. The cost of FDA approved PCa diagnostics in the USA range from USD 9500 to 3700 per scan. Lack of sustained efforts to prepare PCa diagnostics have hampered affordable healthcare in India.

Therefore, developing new and indigenous radiopharmaceuticals that can be protected across the world for PCa. In this context, very little research progress has been achieved in India. In spite of this setback, for the past several years there have been some advances in prostate cancer diagnosis and radio therapy using reported and patented PSMA agents such as ⁶⁸Ga-PSMA-HBED-CC (PSMA-11) and 177Lu-PSMA-617 in clinical scenario for Indian patients. This will restrict us to play a significant role in the global arena for improving the quality of life of ailing patients from prostate malignancy.

Earlier, in 1996, FDA had approved the first diagnostic agent called ProstaScint (111In-labeled anti-PSMA monoclonal antibody) for detection of soft tissue metastases in patients with prostate cancer. However, there has been limited clinical success using this monoclonal antibody agent, because of its long circulating plasma half-life, low permeability in solid tumors, particularly for detection of metastatic disease to bone, high production cost (USD 9500), low shelf life, longer scanning sessions (˜four to five days) and possible immunoreaction. In 2012, FDA approved Choline 11C for PET imaging, but Choline 11C's short half-life limits its use to medical centers with on-site production capability. In the mid-2016, the U.S. Food and Drug Administration approved Axumin (USD 3700 per scan), a PET diagnostic agent derived from synthetic fluorocyclobutane amino acid, for detection of prostate carcinoma, the uptake of which is based on over-expression of amino acid transporters in PCa. This imaging agent is generally employed to detect suspected recurrent prostate cancer based on elevated prostate specific antigen (PSA) levels following treatment of initial disease.

The FDA approved imaging agents are very expensive, requires special facilities and unaffordable in the Indian clinical scenario whereas the diagnostics mentioned in this discovery are indigenous, cost effective, specific to PCa malignancy, short serum clearance time and few scanning sessions.

The cost of FDA approved PCa diagnostics are very expensive, imported and costs approximately USD 9500 to 3700 per scan. One of the biggest drawbacks of using patented agents will restrict us to play a significant role in the global arena for improving the quality of life of ailing patients from prostate malignancy. Developing new and indigenous radiopharmaceuticals that can be protected across the world for PCa without any issue is one of goal. The expected cost of our product is 1/00 to 1/50 times less expensive than that available in the international market.

FDA approved 111In-labeled monoclonal antibody (mAb), ProstaScint, (111In-labeled anti-PSMA monoclonal antibody) can only detect soft tissue metastases in patients with prostate cancer and fails for detection of solid tumors. Further mAb has long circulating plasma half-life, low permeability in solid tumors, particularly for detection of metastatic disease to bone, low shelf life (1-month at 4 degree), needs longer scanning sessions (˜four to five days) and suffers from human immunoreaction. The embodiment described in the present disclosure can detect solid tumors, has long shelf life (6-months at 4 degree), needs 2 h scan session and doesn't suffer from antigen-antibody reactions.

The FDA approved imaging agents requires special facilities and unaffordable in the Indian clinical scenario whereas the diagnostics mentioned in this discovery are indigenous, cost effective and doesn't require special facilities like cyclotron.

In the present disclosure the aforementioned drawbacks are overcome by discovery of new small molecule targeted NIR imaging agents for surgical removal of tumors, treating incurable mCRPC, production of diagnostics and therapeutics, inhibitors of neurodegenerative diseases that are affordable to Indian population as well as to compete in the world market.

In the present disclosure, bio-imaging tool specifically bind with high affinity to cancer cells overexpressing PSMA protein, allowing clear visibility of the exact tumor margins to be excised. Additionally, our newly developed inhibitors or ligands would be used for targeted drug delivery of radioisotopes such as ^{99m}Tc, ⁶⁸Ga, ⁶⁴Cu, ¹⁸F and ¹⁷⁷Lu for diagnosis and treatment of malignancy and neurodegenerative diseases which over-express PSMA protein.

Thus, it is desired to address the above-mentioned disadvantages or other shortcomings or at least provide a useful alternative.

The present disclosure herein solves the above problems by design of several small molecule inhibitors or ligands for diagnosis and treatment of cancers such as prostate, brain, breast, etc., and other neurodegenerative diseases.

### OBJECT OF INVENTION

The principal object of the embodiments herein is to provide a compound and method for diagnosis and treatment of malignancy arising out of prostate, brain, breast, bladder tissues and few of the neurodegenerative diseases like schizophrenia and ALS.

### SUMMARY OF INVENTION

Accordingly embodiments herein disclose a compound and method of small molecule inhibitors or ligands for diagnosis and treatment of cancers such as prostate, brain, breast, etc., and other neurodegenerative diseases. Rational structural design of several small molecule inhibitors based on binding with PSMA protein using in silico study. Design and execution of simple, mild and high yielding chemical synthetic strategy for the preparation of small molecules ligands or inhibitors. Strategy for introduction of fluorescent imaging moieties or cargos (anticancer drugs) using differentially protected lysine amino acid in a continuous process through solid phase peptide synthesis during the preparation of ligand conjugates. Sequential introduction of hydrophilic and hydrophobic amino acids by targeting first, second and third hydrophobic pockets present in the PSMA protein along with chelation moieties based on molecular docking studies and linker length calculations. Selective targeting of PSMA protein expressed on several cancers and neurodegenerative diseases. Preparation of targeting ligands in an affordable manner by a cost-effective process. Tagging of near-infra red fluorescent molecules or cargos for diagnosis, intra-operative surgery and therapy of PCa. Enhanced binding affinity to PSMA protein and tagging of radioisotopes for treatment of metastatic castration resistant prostate cancer (mCRPC) for which no cure is known.

In an embodiment of the present disclosure, there is disclosed a conjugate comprising: a) a ligand; b) a spacer; and c) a drug; wherein the ligand is a compound of Formula I

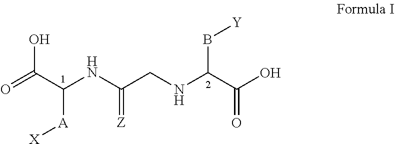

and stereoisomers thereof, wherein A and B are independently selected from a group consisting of hydrogen, optionally substituted C₁-C₇ alkyl, and optionally substituted aryl groups; X and Y are selected from the groups comprising of —H, —OH, and —COOH groups, Z is one of O or S groups. In another embodiment of the present disclosure, A and B are independently selected from a group consisting of hydrogen, C₁-C₃ alkyl, and aryl groups; X and Y are selected from the groups comprising of —H, —OH, and —COOH groups, Z is an O group. In another embodiment of the present disclosure, the ligand is at least one selected from

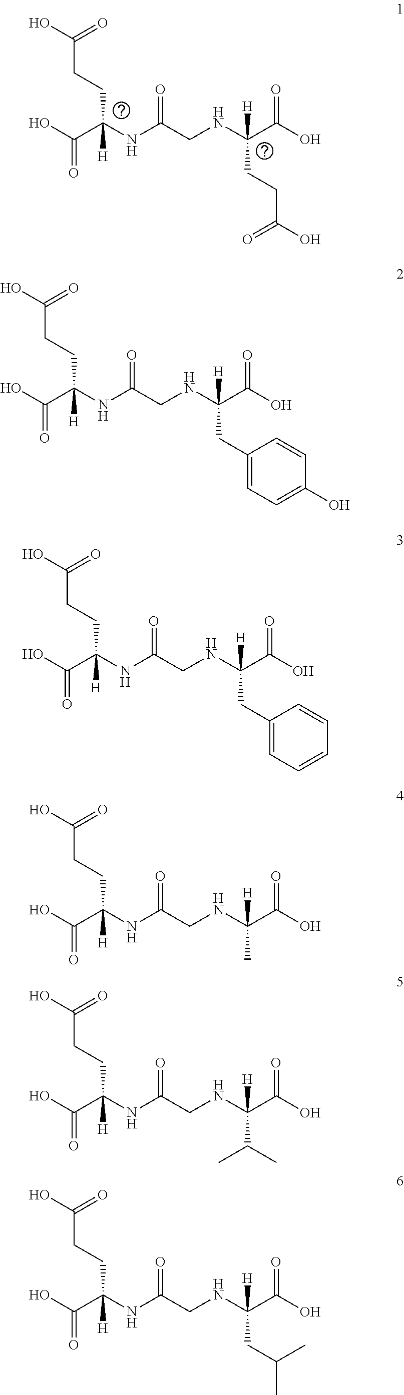

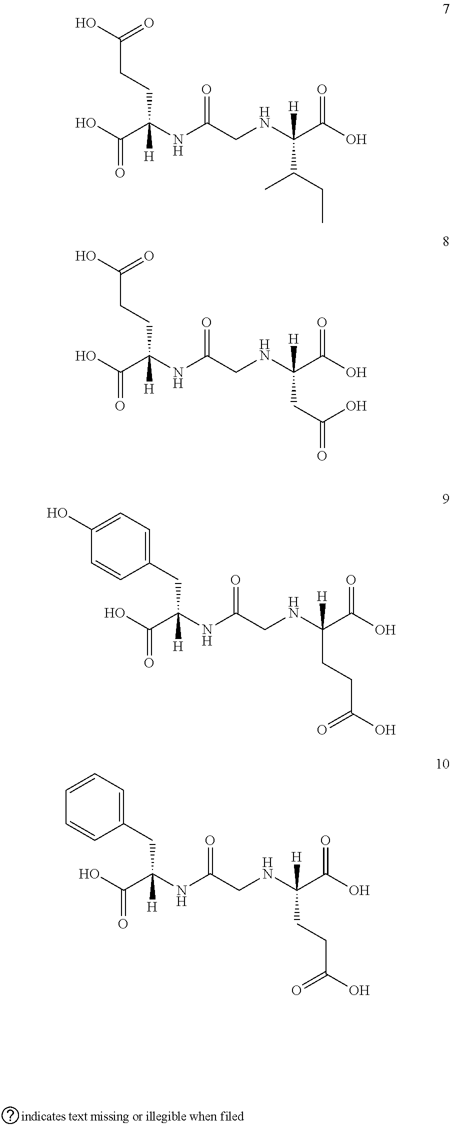

The components of the conjugates described herein may contain one or more chiral centers or may otherwise be capable of existing as multiple stereoisomers. It is to be understood that in one embodiment, the conjugates described herein are not limited to any particular stereochemical requirement, and may be optically pure, or may be any of a variety of stereoisomeric mixtures, including racemic and other mixtures of enantiomers, other mixtures of diastereomers, and the like. It is also to be understood that such mixtures of stereoisomers may include a single stereochemical configuration at one or more chiral centers, while including mixtures of stereochemical configuration at one or more other chiral centers. In an embodiment of the present disclosure, there is disclosed a conjugate of Formula I, wherein stereochemical configuration of the stereocenter 1 and 2 of the compound of Formula I is of S configuration.

In an embodiment of the present disclosure, there is disclosed a conjugate comprising: a) a ligand; b) a spacer; and c) a drug; wherein the ligand is a compound of Formula I, and wherein the spacer is a peptide comprising 2-20 amino acids. In an embodiment of the present disclosure, the spacer comprises at least two phenylalanine residues, each of which is optionally substituted, or aminocaprylic acid, or both.

In an embodiment of the present disclosure, there is disclosed a conjugate comprising: a) a ligand; b) a spacer; and c) a drug; wherein the drug is at least one of an imaging agent, anticancer drug or a radionuclide. In an embodiment of the present disclosure, the imaging agent is a fluorescent agent. Fluorescent agents include Oregon Green fluorescent agents, including but not limited to Oregon Green 488, Oregon Green 514, and the like, AlexaFluor fluorescent agents, including but not limited to AlexaFluor 488, AlexaFluor 647, and the like, fluorescein, and related analogs, BODIPY fluorescent agents, including but not limited to BODIPY FI, BODIPY 505, and the like, rhodamine fluorescent agents, including but not limited to tetramethylrhodamine, and the like, DyLight fluorescent agents, including but not limited to DyLight 680, DyLight 750, DyLight 800, and the like, CW 800, Texas Red, phycoerythrin, and others.

In an embodiment, the imaging agent is a radioactive isotope of a metal coordinated to a chelating group, where the radioactive isotope is selected from a group consisting of ^{99m}Tc, ⁶⁸Ga, ¹⁸F and ¹⁷⁷Lu. The chelating group has a formula II

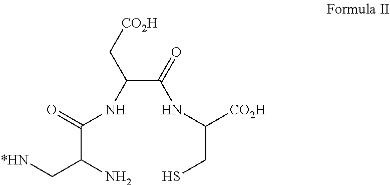

wherein * indicates the site of attachment to the spacer

In another aspect, the imaging agent is a PET imaging agent, or a FRET imaging agent. PET imaging agents include ¹⁸F, ⁶⁴Cu, ⁶⁸Ga, and the like. FRET imaging agents include ⁶⁴Cu, ⁶⁸Ca, and the like. In another aspect, the therapeutic agent is a cytotoxic compound. The cytotoxic compounds described herein operate by any of a large number of mechanisms of action. Generally, cytotoxic compounds disrupt cellular mechanisms that are important for cell survival and/or cell proliferation and/or cause apoptosis.

The drug (D) can be any therapeutic agent capable of modulating or otherwise modifying cell function, including pharmaceutically active compounds. Suitable therapeutic agents can include, but are not limited to: peptides, oligopeptides, retro-in verso oligopeptides, proteins, protein analogs in which at least one non-peptide linkage replaces a peptide linkage, apoproteins, glycoproteins, enzymes, coenzymes, enzyme inhibitors, amino acids and their derivatives, receptors and other membrane proteins; antigens and antibodies thereto; haptens and antibodies thereto; hormones, lipids, phospholipids, liposomes; toxins; cancer drugs including therapeutic agents.

Further, the drug (D) can be any drug known in the art which is cytotoxic, enhances tumor permeability, inhibits tumor cell proliferation, promotes apoptosis, decreases anti-apoptotic activity in target cells, enhances an endogenous immune response directed to the pathogenic cells, or is useful for treating a disease state caused by any type of cancer cells. Drugs (D) suitable for use in accordance with this invention include adrenocorticoids and corticosteroids, alkylating agents, antiandrogens, antiestrogens, androgens, aclamycin and aclamycin derivatives, estrogens, antimetabolites such as cytosine arabinoside, purine analogs, pyrimidine analogs, and methotrexate, busulfan, carboplatin, chlorambucil, cisplatin and other platinum compounds, taxanes, such as tamoxiphen, taxol, paclitaxel, paclitaxel derivatives,

Taxotere®, and the like, maytansines and analogs and derivatives thereof, cyclophosphamide, daunomycin, doxorubicin, rhizoxin, T2 toxin, plant alkaloids, prednisone, hydroxyurea, teniposide, mitomycins, discodermolides, microtubule inhibitors, epothilones, tubulysins, and analogs and derivatives thereof, cyclopropyl benz[e]indolone, secocyclopropyl benz[e]indolone, 0-Ac-secocyclopropyl benz[e]indolone, bleomycin and any other antibiotic, nitrogen mustards, nitrosureas, vinca alkaloids, vincristine, vinblastine, and analogs and derivative thereof such as deacetylvinblastine monohydrazide, colchicine, colchicine derivatives, allocolchicine, thiocolchicine, trityl cysteine, Halicondrin B, dolastatins such as dolastatin 10, amanitins such as a-amanitin, camptothecin, irinotecan, and other camptothecin derivatives thereof, geldanamycin and geldanamycin derivatives, estramustine, nocodazole, MAP4, colcemid, inflammatory and proinflammatory agents, peptide and peptidomimetic signal transduction inhibitors, and any other art-recognized drug.

Illustrative drugs and other therapeutic agents are described in U.S. Patent Application Publication Nos. US-2005-0002942-A1, US-2001-0031252-A1, and US-2003-0086900-A1. Illustrative imaging agents and diagnostic agents are described in U.S. Patent Application Publication No. US-2004-0033195-A1 and International Patent Application Publication No. WO 03/097647. The disclosures of each of the foregoing patent application publications are incorporated herein by reference.

In an embodiment of the present disclosure, the conjugate is a molecule of formula III

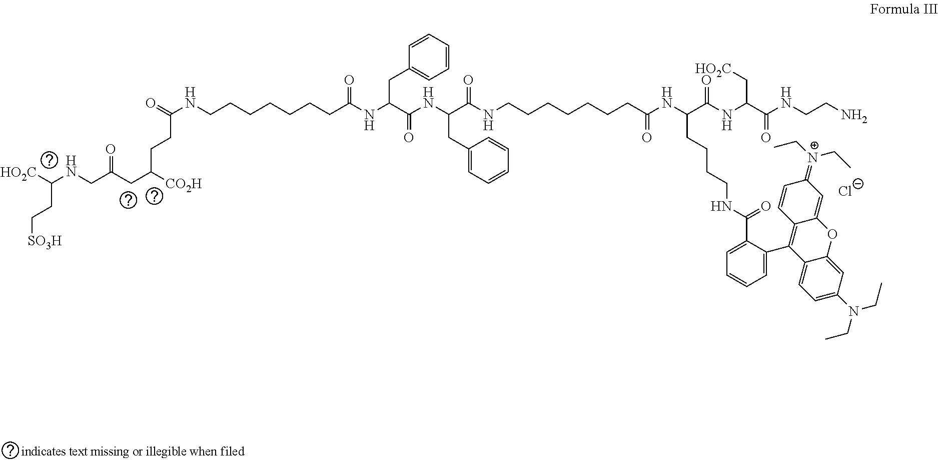 [SMILES: CCN(CC)c1ccc2c(-c3ccccc3C(=O)NCCCCC(NC(=O)CCCCCCCNC(=O)C(Cc3ccccc3)NC(=O)C(Cc3ccccc3)NC(=O)CCCCCCCNC(=O)CCC(CC(=O)CNC(CCS(=O)(=O)O)C(=O)O)C(=O)O)C(=O)NC(CC(=O)O)C(=O)NCCN)c3ccc(=[N+](CC)CC)cc-3oc2c1 ; [Cl-]]

In another embodiment of the present disclosure, the conjugate is a molecule of formula IV Formula IV

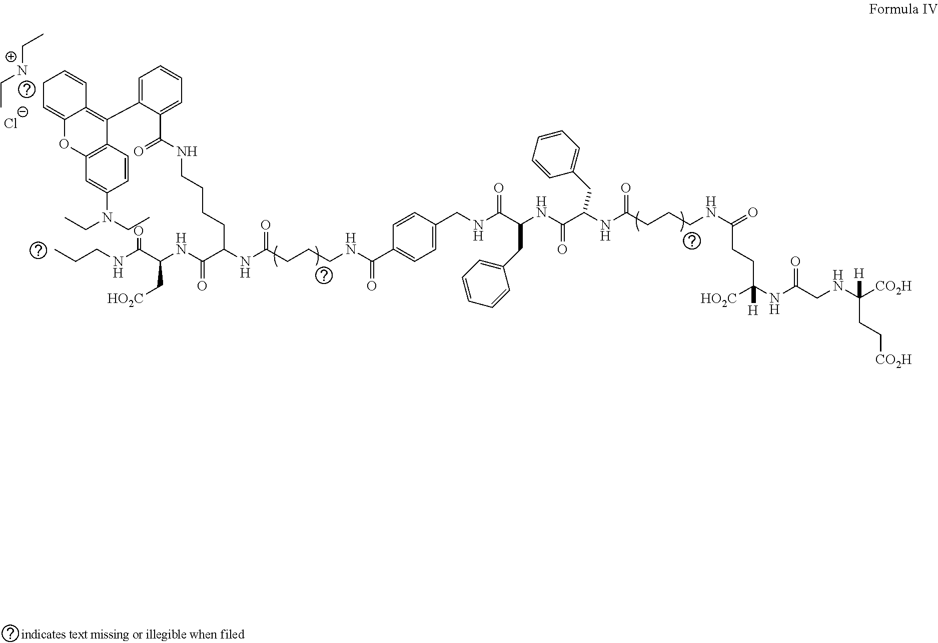

In yet another embodiment of the present disclosure the conjugate is a molecule of formula V

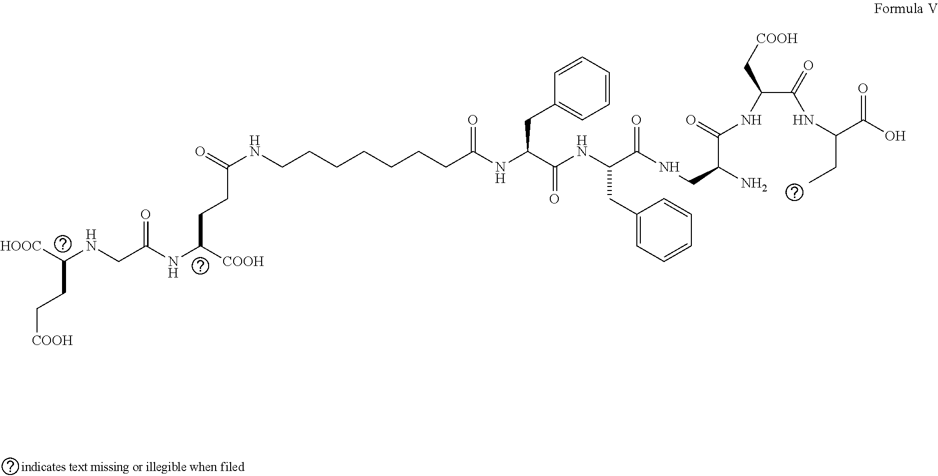 [SMILES: CCC(NC(=O)[C@H](CC(=O)O)NC(=O)[C@@H](N)CNC(=O)[C@H](Cc1ccccc1)NC(=O)[C@H](Cc1ccccc1)NC(=O)CCCCCCCNC(=O)CC[C@H](NC(=O)CN[C@@H](CCC(=O)O)C(=O)O)C(=O)O)C(=O)O]

In an embodiment of the present disclosure, the conjugate is a molecule of formula VI

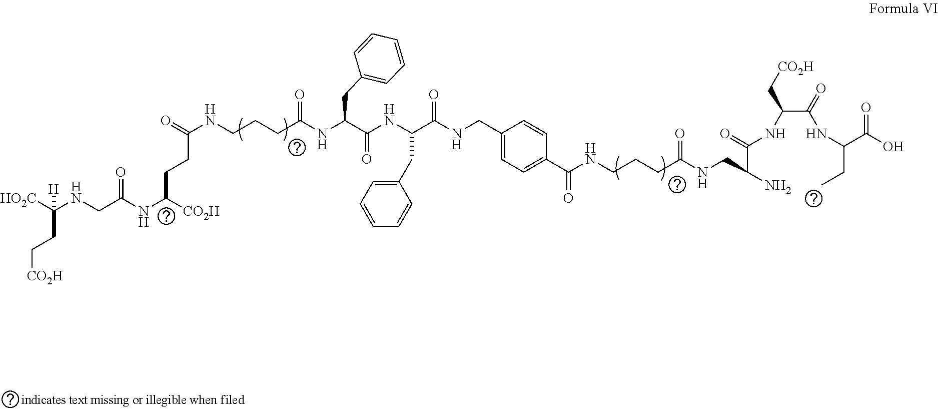

In an embodiment of the present disclosure, the conjugate is a molecule of Formula VII. The molecule is an arene DOTA conjugate used for PCa diagnosis.

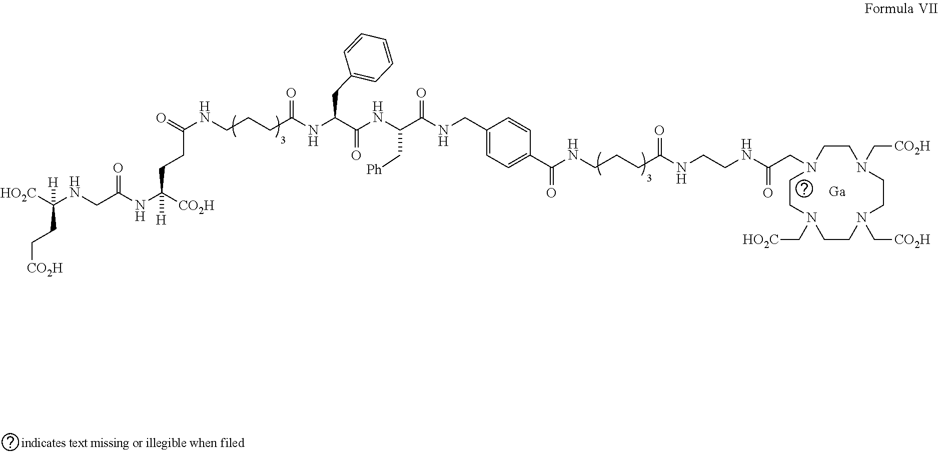

In each of the foregoing and following embodiments, it is to be understood that the formulae include and represent not only all pharmaceutically acceptable salts of the compounds in the conjugates, but also include any and all hydrates and/or solvates of the formulae. It is appreciated that certain functional groups, such as the hydroxy, amino, and like groups form complexes and/or coordination compounds with water and/or various solvents, in the various physical forms of the compounds in the conjugates described herein. Accordingly, the formulae described herein are to be understood to include and represent those various hydrates and/or solvates. In each of the foregoing and following embodiments, it is also to be understood that the formulae include and represent each possible isomer, such as stereoisomers and geometric isomers, both individually and in any and all possible mixtures. In each of the foregoing and following embodiments, it is also to be understood that the formulae include and represent any and all crystalline forms, partially crystalline forms, and non-crystalline and/or amorphous forms of the compounds in the conjugates described herein.

In another embodiment, pharmaceutical compositions are described herein, where the pharmaceutical composition includes the conjugates described herein in amounts effective to treat diseases and disease states, diagnose diseases or disease states, and/or image tissues and/or cells that are associated with pathogenic populations of cells expressing or over expressing PSMA. Illustratively, the pharmaceutical compositions also include one or more carriers, diluents, and/or excipients. Excipients may serve as a diluent, and can be solid, semi-solid, or liquid materials, which act as a vehicle, carrier, or medium for the active ingredient. Thus, the formulation compositions can be in the form of suspensions, emulsions, solutions, sterile injectable solutions, and sterile packaged powders. The compositions may contain anywhere from about 0.1% to about 99.9% active ingredients, depending upon the selected dose and dosage form. Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxybenzoates; sweetening agents; and flavoring agents. The compositions can be formulated so as to provide quick, sustained or delayed release of the active ingredient after administration to the patient by employing procedures known in the art. It is appreciated that the carriers, diluents, and excipients used to prepare the compositions described herein are advantageously GRAS (generally regarded as safe) compounds. Examples of emulsifying agents are naturally-occurring gums (e.g., gum acacia or gum tragacanth) and naturally occurring phosphatides (e.g., soybean lecithin and sorbitan monooleate derivatives). Examples of antioxidants are butylated hydroxy anisole (BHA), ascorbic acid and derivatives thereof, tocopherol, and derivatives thereof, butylated hydroxy anisole, and cysteine. Examples of preservatives are parabens, such as methyl or propyl p-hydroxybenzoate, and benzalkonium chloride. Examples of humectants are glycerin, propylene glycol, sorbitol, and urea. Examples of penetration enhancers are propylene glycol, DMSO, triethanolamine, N,N-dimethylacetamide, N,N-dimethylformamide, 2-pyrrolidone and derivatives thereof, tetrahydrofurfuryl alcohol, and AZONE. Examples of chelating agents are sodium EDTA, citric acid, and phosphoric acid. Examples of gel forming agents are CARBOPOL, cellulose derivatives, bentonite, alginates, gelatin and polyvinylpyrrolidone. Examples of ointment bases are beeswax, paraffin, cetyl palmitate, vegetable oils, sorbitan esters of fatty acids (Span), polyethylene glycols, and condensation products between sorbitan esters of fatty acids and ethylene oxide (e.g., polyoxyethylene sorbitan monooleate (TWEEN).

The term “therapeutically effective amount” as used herein, refers to that amount of active conjugate that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. The term “administering” as used herein includes all means of introducing the conjugates and compositions described herein to the patient, including, but not limited to intravenous (iv), intramuscular (im), subcutaneous (sc), transdermal, and the like. The conjugates and compositions described herein may be administered in unit dosage forms and/or formulations containing conventional nontoxic pharmaceutically-acceptable carriers, adjuvants, and vehicles.

An embodiment relates to a conjugate having a formula: D-E-F wherein D comprises AAPT ligand or a derivative thereof and having formula I

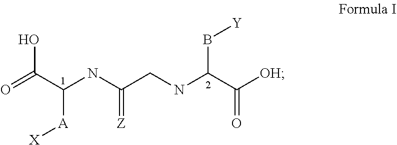

is a spacer comprising a chain of amino acid or its derivatives comprising AA1, AA2, AA3 and/or AA4;
F is a chelating group comprises formula II

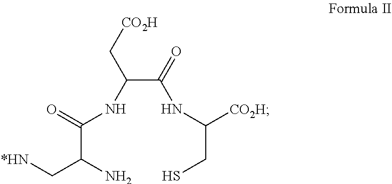

wherein A and B of formula I are independently selected from a group consisting of hydrogen, optionally substituted C₁-C₇ alkyl, and optionally substituted aryl groups; X and Y are selected from the groups comprising of —H, —OH, and —COOH groups, and Z is one of O or S groups;
wherein formula I comprises polar charged or polar uncharged amino acid or both;
wherein more than two of AA1, AA2, AA3 and/or AA4 are present; wherein * indicates the site of attachment to the spacer;
wherein the conjugate is configured to have binding affinity≤60 nM.

In an embodiment, formula I comprises polar negatively charged amino acid.

In an embodiment, AA1 comprises trans-4-aminomethylcyclohexanecarboxylic acid or 2-naphthylalanine.

In an embodiment, AA2 comprises 2-naphthylalanine or trans-4-aminomethylcyclohexanecarboxylic acid.

In an embodiment, AA3 comprises 2-naphthylalanine or phenylalanine or NH2-(CH2)2-PEG2/3-(CH2)2CO2H or NH2-(CH2)7-CO2H.

In an embodiment, AA4 comprises NH2-(CH2)2-PEG2/3-(CH2)2CO2H.

In an embodiment, AA4 comprises Polyethylene Glycol 3 (PEG3).

In an embodiment, the conjugate comprises AAPT ligand conjugated arene chelating linker for delivery of ^{99m}Tc radioisotope comprises:

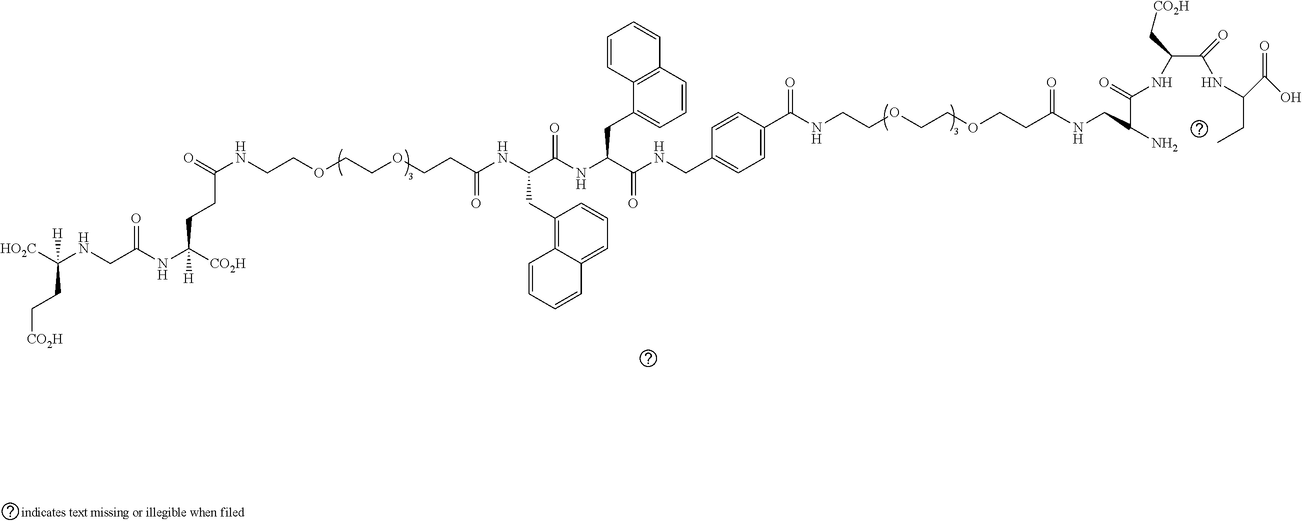

In an embodiment, the conjugates having formula:

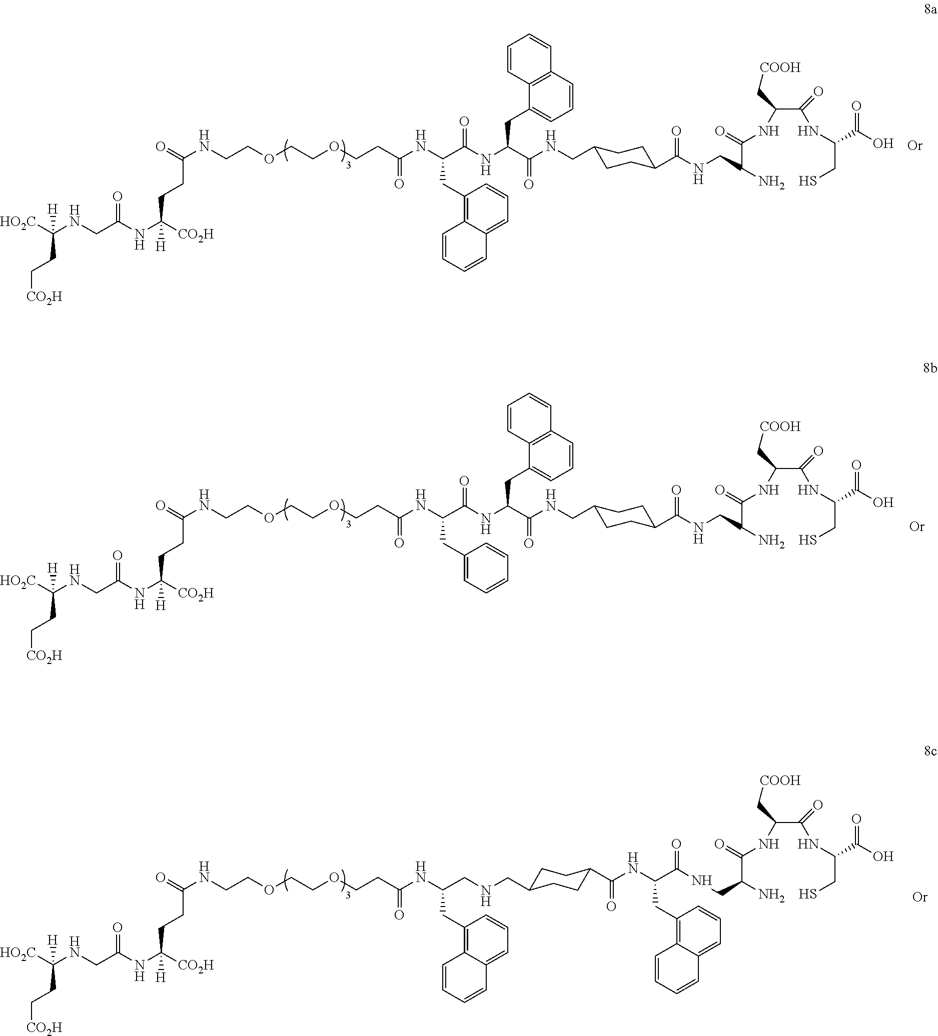

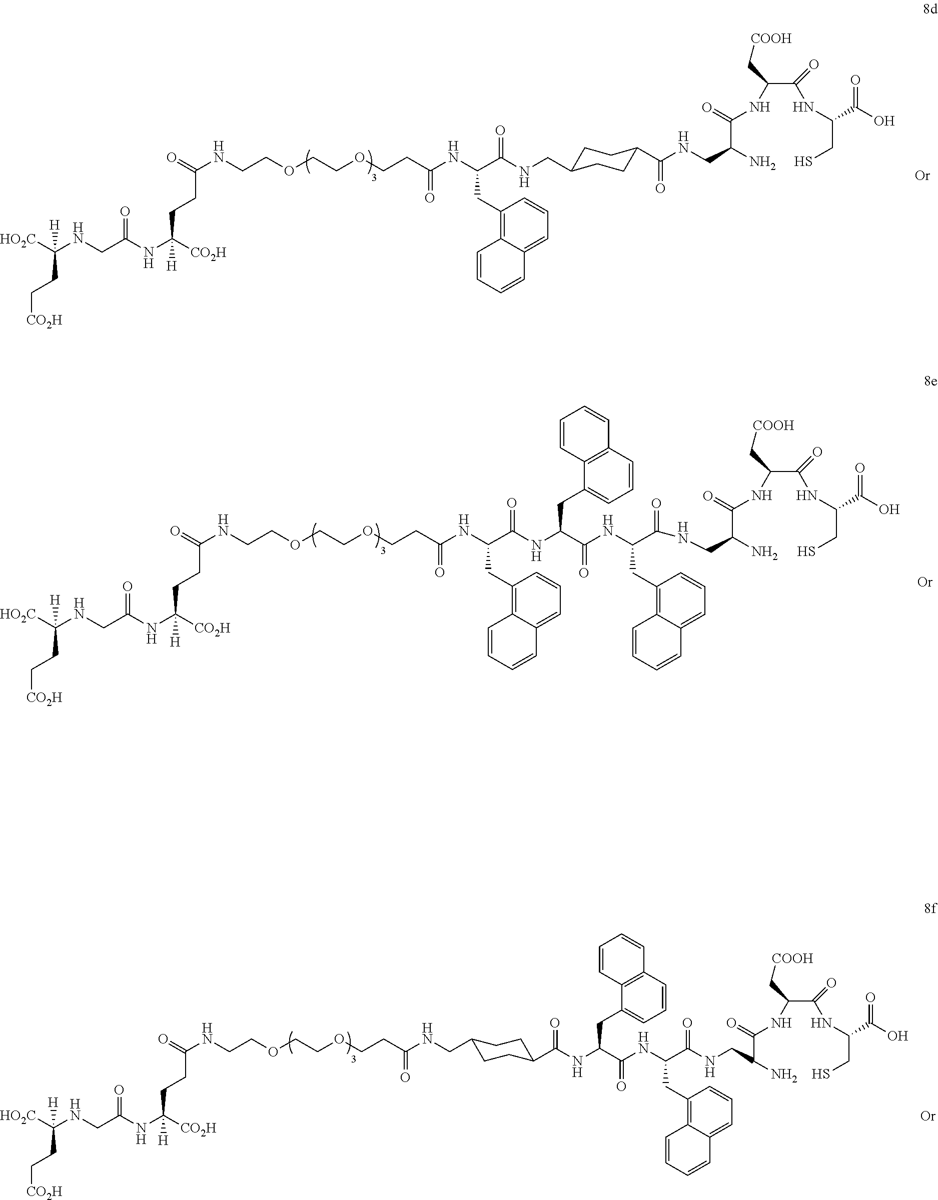

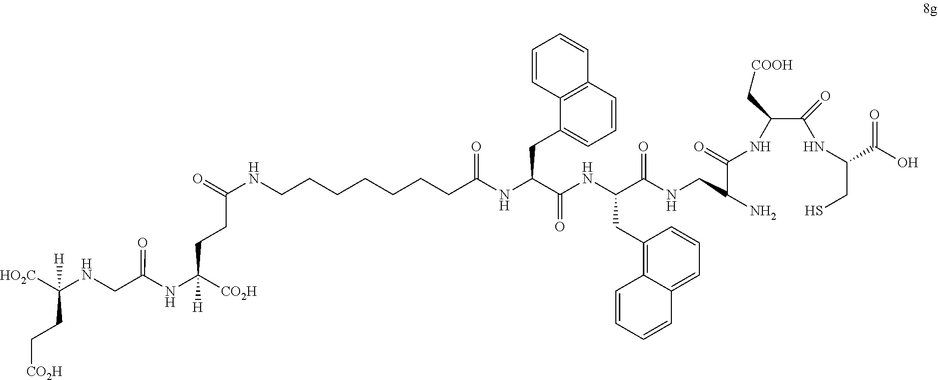

In an embodiment, the conjugate is configured to be used for targeted drug delivery of radioisotopes for diagnosis and treatment of malignancy and neurodegenerative diseases where PSMA protein is overexpressed.

In an embodiment, the amino acid residues of E are optionally substituted.

An embodiment relates to a method of preparation of precursor of AAPT ligand as following:
(a) stirring a solution of protected amino acids (1 equiv.) and NaHCO₃ (1.2 equiv.) in CH₂Cl₂:H₂O (5v:0.5v or 1v:1v) and cooling to 0° C. to get a reaction mixture;
(b) adding α-Chloroacetyl chloride (1.2 equiv.) dropwise over a period of 15 minutes at 0° C. to the reaction mixture of step (a) and stirring the reaction mixture for 1 h by allowing the temperature to raise to room temperature and further stirring for 1 h or 2 h;
(c) diluting the reaction mixture of step (b) with water and dichloromethane and separating an organic layer and washing it with sodium bicarbonate solution;
(d) drying the organic layer of step (c) over anhydrous sodium sulfate, filtering and concentrating under reduced pressure to get (S)-tert-Butyl 2-(2-chloroacetamido)-3-phenyl ester derivative in 78-96% yields;
(e) reacting (S)-tert-Butyl 2-(2-chloroacetamido)-3-phenyl ester derivative of step (d) with an amine derivative in the presence of potassium iodide and K₂CO₃ in refluxing dry THF overnight to get precursors of AAPT ligands in 82-94% yields;
(f) characterizing the precursors of AAPT ligand of using various spectroscopic techniques.

In an embodiment, the method of preparation of precursor of AAPT ligand further comprises a method of solid phase synthesis of AAPT ligand conjugated with a spacer is as following:
(a) swelling of H-Cys-2-ClTrt resin (0.80-0.100 g) in a resin beads in CH₂Cl₂ (5-10 mL) for 30 minutes by bubbling nitrogen and after draining CH₂Cl₂, the resin is swelled once again with DMF (5 mL×3) thrice for 15 minutes each and then (i) adding Fmoc-Asp(O^{t}Bu)-OH (90-100 mg), PyBOP (110-120 mg) and DIPEA (0.1-0.2 mL) in DMF (0.5 mL) to the peptide vessel and continuing the coupling reaction for 6 h and then washing the resin with DMF thrice following by isopropanol thrice to perform a reaction; (ii) confirming the completion of the reaction by performing Kaiser test and then adding 20% Piperidine in DMF at room temperature and bubbling the resin beads for 30 min;
(b) adding Fmoc-diaminopropionic (DAP) acid, PyBOP and DIPEA in DMF for 6 h and then adding 20% Piperidine in DMF at room temperature and bubbling the resin beads for 30 min;
(c) adding Fmoc-AA1-OH, PyBOP and DIPEA in DMF for 6 h and then adding 20% Piperidine in DMF at room temperature and bubbling the resin beads for 30 min;
(d) adding Fmoc-AA₂-OH, PyBOP and DIPEA in DMF for 6 h and then adding 20% Piperidine in DMF at room temperature and bubbling the resin beads for 30 min;
(e) adding Fmoc-AA₃-OH, PyBOP and DIPEA in DMF for 6 h and then adding 20% Piperidine in DMF at room temperature and bubbling the resin beads for 30 min;
(f) adding Fmoc-AA₄-OH, PyBOP and DIPEA in DMF for 6 h and then adding 20% Piperidine in DMF at room temperature and bubbling the resin beads for 30 min;
(g) adding AAPT(O^{t}Bu)₃-OH, PyBOP, and DIPEA in DMF to the peptide vessel of step (f) for 6 h;
(h) adding 5 ml of a mixture of TFA to the resin beads and bubbling nitrogen gas through resin beads for 30 min and then 2.5 ml of the mixture of TFA for 15 min twice and collecting a mother liquor in a round bottom flask and then evaporating the mixture of TFA from the mother liquor to form concentrated viscous mixture;
(i) precipitating the AAPT ligand conjugated with the spacer from the concentrated viscous mixture of step (h) in ice cold diethylether.

In an embodiment, a mixture of TFA comprises TFA/H₂O/TIPS/EDT (92.5:2.5:2.5:2.5) or TFA/H₂O/TIPS (95:2.5:2.5).

In an embodiment. (S)-tert-Butyl 2-(2-chloroacetamido)-3-phenyl ester derivative is any of the 13a-c (FIG. 21).

In an embodiment, an amine derivative in the method is 14a-j (FIG. 21).

An embodiment relates to a conjugate comprising:
a) a ligand;
b) a spacer; and
c) a chelating ligand
wherein the ligand is a compound of Formula I

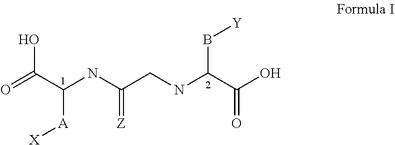

and stereoisomers thereof, wherein A and B are independently selected from a group consisting of hydrogen, optionally substituted C₁-C₇ alkyl, and optionally substituted aryl groups; X and Y are selected from the groups comprising of —H, —OH, and —COOH groups, and Z is one of O or S groups;
wherein the conjugate is a AAPT-PCa DOTA bioconjugate.

In an embodiment, the spacer is a peptide comprising at least 2-20 amino acids.

In an embodiment, the drug is at least one of imaging agents, anticancer drug or a radionuclide.

In an embodiment, the conjugate comprises AAPT-PCa DOTA bioconjugate having formula:

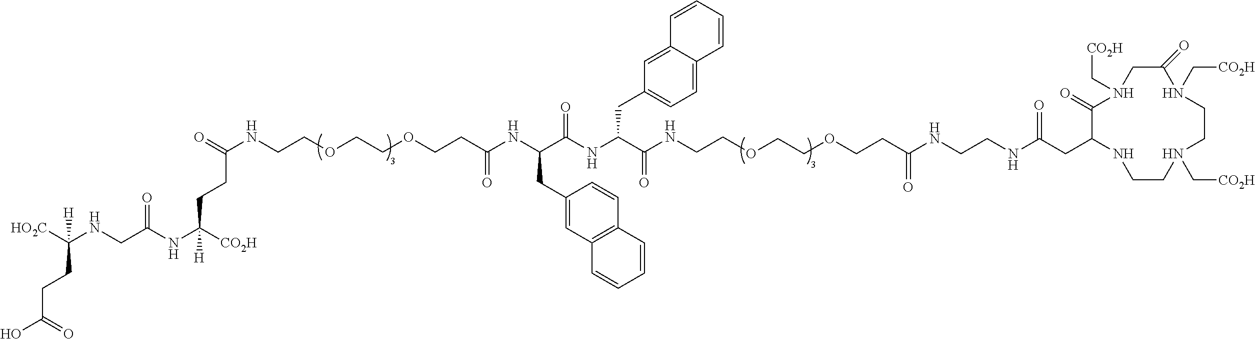

In an embodiment, a method of solid phase synthesis of AAPT-PCa DOTA bioconjugate, for delivering of radioisotopes comprising:
(a) swelling of Universal NovaTag resin in resin beads in a peptide vessel in 20% Piperdine in DMF at room temperature and bubbling the resin beads for 30 min and then adding Fmoc-NH—(PEG)₃-CO₂H, PyBOP and DIPEA in DMF to the peptide vessel and continuing the coupling reaction for 6 h and washing the resin with DMF thrice following by isopropanol thrice to perform a reaction;
(b) confirming the completion of the reaction by performing Kaiser test and then adding 20% Piperdine in DMF at room temperature and bubbling the resin beads for 30 min and adding Fmoc-2-Nal-OH twice, PyBOP and DIPEA in DMF for 6 h;
(c) confirming the completion of the reaction by performing Kaiser test and then adding 20% Piperdine in DMF at room temperature and bubbling the resin beads for 30 min and adding Fmoc-NH—(PEG)₃-COOH PyBOP and DIPEA in DMF for 6 h;
(d) adding 20% Piperdine in DMF at room temperature and bubbling the resin beads for 30 min and adding AAPT(O^{t}Bu)₃-OH, PyBOP, and DIPEA in DMF to the peptide vessel of step (c) for 6 h;
(e) adding 1M HOBt in DCM/TFE (1:1) for 10 min at room temperature and then adding DOTA-tris(tert-butyl ester), PyBOP and DIPEA in DMF for 6 h;
(f) adding 5 ml of a mixture of TFA to the resin beads and bubbling nitrogen gas through resin beads for 30 min and then 5 ml of the mixture of TFA for 5 min twice and collecting a mother liquor in a round bottom flask;
(g) evaporating the mixture of TFA from the mother liquor to form concentrated viscous mixture;
(h) precipitating the AAPT-PCa DOTA bioconjugate from the concentrated viscous mixture of step (g) in ice cold diethylether.

In an embodiment, the method for delivering of radioisotopes comprising 64Cu, 68Ga and 177Lu as radioisotopes.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF FIGURES

This method is illustrated in the accompanying drawings, throughout which like reference letters indicate corresponding parts in the various figures. The embodiments herein will be better understood from the following description with reference to the drawings, in which:

FIG. 1a illustrates four components or modules, according to an embodiment as disclosed herein;

FIG. 1b illustrates molecular modelling method for the inhibitors, according to an embodiment as disclosed herein;

FIG. 1c illustrates chemical synthesis of designed inhibitor, according to an embodiment as disclosed herein;

FIG. 1d illustrates design of bioconjugate handle in the linker for attaching cargos such as near-infrared fluorescent agents or anticancer drugs, according to an embodiment as disclosed herein;

FIG. 1e illustrates design of hydrophobic peptidic spacer for enhanced binding affinity to PSMA protein along with chelating moieties for tagging nuclear radioisotopes or nanoparticles that can be used for imaging and therapy, according to an embodiment as disclosed herein;

FIG. 2 illustrates design of lead aminoacetamide peptidomimetic 1 for PSMA enzyme inhibition, according to an embodiment as disclosed herein;

FIG. 3 illustrates design of library of peptidomimetics based on amino acetamide scaffold, according to an embodiment as disclosed herein;

FIG. 4 illustrates molecular docking study of JB7 with GCPII protein (PDB id-4NGM), according to an embodiment as disclosed herein;

FIG. 5 illustrates molecular docking study of ligand 1 designated as AAPT at GCPII active cavity (PDB id-4NGM), according to an embodiment as disclosed herein;

FIG. 6 illustrates computational docking of aminoacetamide inhibitor 3 in the active site of GCPII (PDB=4NGM), according to an embodiment as disclosed herein;

FIG. 7 illustrates hydrogen bonding interactions of aminoacetamide inhibitor 2 in the active site of GCPII (PDB=4NGM) in comparison to 3, according to an embodiment as disclosed herein;

FIG. 8 illustrates synthesis of precursors 15a-j for acetamide based GCPII ligands 1-10, according to an embodiment as disclosed herein;

FIG. 9 illustrates list of acetamide based GCPII inhibitor precursors 15a-j, according to an embodiment as disclosed herein;

FIG. 10 illustrates synthesis of acetamide based GCPII inhibitors 1-3 from precursors 15a-c, according to an embodiment as disclosed herein;

FIG. 11(a) illustrates inhibition curve of AAPT inhibitor 1 against PSMA protein isolated from prostate cancer cell LNCaP SD (n=1), according to an embodiment as disclosed herein;

FIG. 11(b) illustrates inhibition curve of PSMA inhibitors 2 against PSMA protein isolated from prostate cancer, LNCaP cells SD (n=3), according to an embodiment as disclosed herein;

FIG. 11(c) illustrates Inhibition curve of PSMA inhibitors 3 against PSMA protein isolated from prostate cancer, LNCaP cells SD (n=3), according to an embodiment as disclosed herein;

FIG. 12 illustrates dose-response curve of standard inhibitor, 2-PMPA, against PSMA protein isolated from prostate cancer cells, LNCaP; error bars represent SD (n=3), according to an embodiment as disclosed herein;

FIG. 13 illustrates solid phase synthetic strategy for preparation of AAPT rhodamine B conjugate 22 AAPT-C17201 reagents and conditions, according to an embodiment as disclosed herein; FIG. 14 illustrates laser scanning confocal microscopy uptake study of PSMA targeted AAPT-C17201 fluorescent conjugate 22 in prostate cancer cell LNCaP at various concentrations, according to an embodiment as disclosed herein;

FIG. 15 illustrates binding affinity constant KD, determination of fluorescent conjugate 22 AAPT-C17201 in prostate cancer cell LNCaP SD (n=3), according to an embodiment as disclosed herein;

FIG. 16 illustrates solid phase synthetic strategy for preparation of AAPT rhodamine B conjugate 25 reagents and conditions, according to an embodiment as disclosed herein;

FIG. 17 illustrates binding of AAPT-arene rhodamine conjugate 25 in PSMA+ LNCaP cells for a range of concentrations plotted against the mean fluorescence intensity to yield a dissociation constant KD of 130 nM, according to an embodiment as disclosed herein;

FIG. 18 illustrates synthesis of AAPT ligand conjugated chelating linker 31 reagents and conditions, according to an embodiment as disclosed herein;

FIG. 19 illustrates synthesis of AAPT ligand conjugated arene chelating linker 34 reagents and conditions, according to an embodiment as disclosed herein;

FIG. 20 illustrates structures of the PSMA scaffolds, glutamate urea heterodimers, glutamate phosphoramidates, 2 (phosphinylmethyl) pentanedioic acid and acetamide derivatives, according to an embodiment as disclosed herein.

FIG. 21 illustrates another method of synthesis of precursors 15a-j of acetamide based GCPII ligands 1-10.

FIG. 22 illustrates general SPPS method for preparation of AAPT_PCa bioconjugates 8a-f with various spacers.

FIG. 23 illustrates general structure of AAPT_PCa bioconjugates 8a-f with various spacers.

FIG. 24 illustrates binding affinity of AAPT-PCa bioconjugate 8a in PSMA+ LNCap cells.

FIG. 25 illustrates binding affinity of AAPT-PCa bioconjugate 8b in PSMA+ LNCap cells.

FIG. 26 illustrates binding affinity of AAPT-PCa bioconjugate 8c in PSMA+ LNCap cells.

FIG. 27 illustrates binding affinity of AAPT-PCa bioconjugate 8d in PSMA+ LNCap cells.

FIG. 28 illustrates binding affinity of AAPT-PCa bioconjugate 8e in PSMA+ LNCap cells.

FIG. 29 illustrates binding affinity of AAPT-PCa bioconjugate 8f in PSMA+ LNCap cells.

FIG. 30 illustrates binding affinity of AAPT-PCa bioconjugate 8g in PSMA⁺ LNCap cells.

FIG. 31 illustrates scheme 2: synthesis of AAPT ligand conjugated arene chelating linker 14.

FIG. 32 illustrates scheme 3: preparation of AAPT-PCa DOTA bioconjugate 20 using SPPS for delivery of radioisotopes such as 68Ga and 177Lu.

### DETAILED DESCRIPTION OF INVENTION

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. Also, the various embodiments described herein are not necessarily mutually exclusive, as some embodiments can be combined with one or more other embodiments to form new embodiments. The term “or” as used herein, refers to a non-exclusive or, unless otherwise indicated. The examples used herein are intended merely to facilitate an understanding of ways in which the embodiments herein can be practiced and to further enable those skilled in the art to practice the embodiments herein. Accordingly, the examples should not be construed as limiting the scope of the embodiments herein.

The accompanying drawings are used to help easily understand various technical features and it should be understood that the embodiments presented herein are not limited by the accompanying drawings. As such, the present disclosure should be construed to extend to any alterations, equivalents and substitutes in addition to those which are particularly set out in the accompanying drawings. Although the terms first, second, etc. may be used herein to describe various elements, these elements should not be limited by these terms. These terms are generally only used to distinguish one element from another.

Accordingly, embodiments describes a compound and method for diagnosis and treatment of malignancy arising out of prostate, brain, breast, bladder tissues and few of the neurodegenerative diseases like schizophrenia and ALS.

In an embodiment, the present disclosure describes a small molecule inhibitors or ligands for diagnosis and treatment of cancers such as prostate, brain, breast, etc., and other neurodegenerative diseases.

The term “alkyl” as used herein includes a chain of carbon atoms, which is optionally branched.

The term “aryl” as used herein includes molecular fragments or radicals comprising an aromatic mono or polycyclic ring of carbon atoms, such as phenyl, naphthyl, and the like.

The term “substituted aryl” as used herein includes molecular fragments or radicals comprising aryl with one or more substituents, such as alkyl, heteroalkyl, halo, hydroxy, amino, alkyl or dialkylamino, alkoxy, alkylsulfonyl, aminosulfonyl, carboxylate, alkoxycarbonyl, aminocarbonyl, cyano, nitro, and the like. It is to be understood that the alkyl groups in such substituents may be optionally substituted with halo.

The term “amino acid” as used herein includes molecular fragments or radicals comprising an aminoalkylcarboxylate, where the alkyl radical is optionally substituted with alkyl, hydroxy alkyl, sulfhydrylalkyl, aminoalkyl, carboxyalkyl, and the like, including groups corresponding to the naturally occurring amino acids, such as serine, cysteine, methionine, aspartic acid, glutamic acid, and the like.

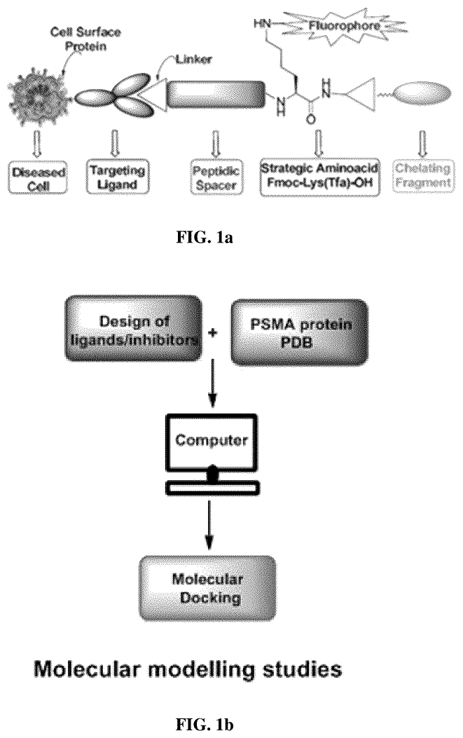

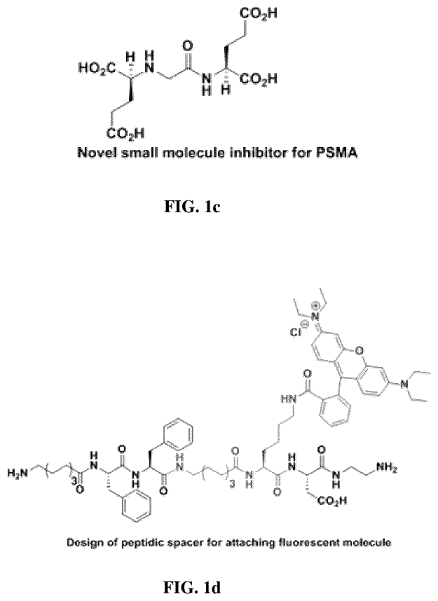

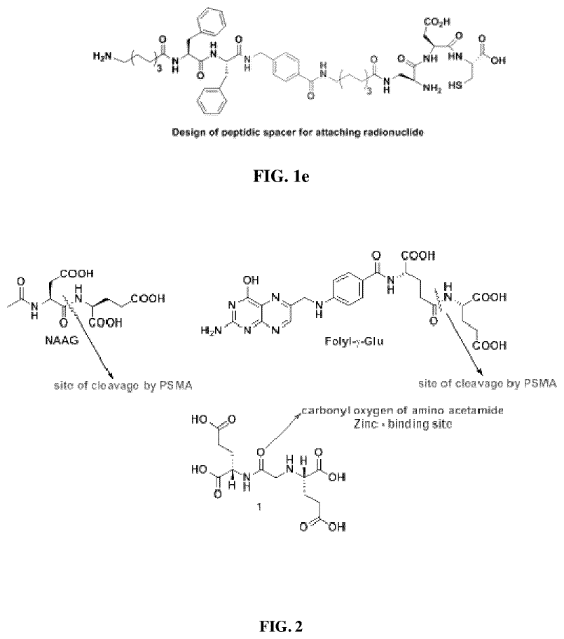

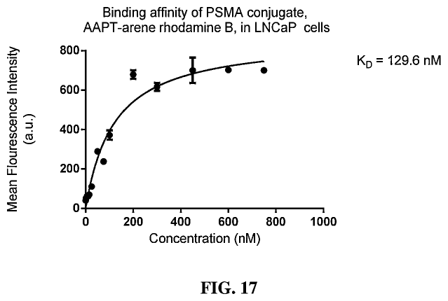

Referring now to the drawings and more particularly to FIGS. 1a through 19 is shown preferred embodiments.

FIG. 1a illustrates four components or modules, according to an embodiment as disclosed herein.

In an embodiment in FIG. 1a, the four components or modules include rational structural design of several small molecule inhibitors based on binding with PSMA protein using in silico study, design and execution of simple, mild and high yielding chemical synthetic strategy for the preparation of small molecules ligands or inhibitors, strategy for introduction of fluorescent imaging moieties or cargos (anticancer drugs) using differentially protected lysine amino acid in a continuous process through solid phase peptide synthesis during the preparation of ligand conjugates and sequential introduction of hydrophilic and hydrophobic amino acids by targeting first, second and third hydrophobic pockets present in the PSMA protein along with chelation moieties based on molecular docking studies and linker length calculations. Selective targeting of PSMA protein expressed on several cancers and neurodegenerative diseases. Preparation of targeting ligands in an affordable manner by a cost-effective process. Tagging of near-infra red fluorescent molecules or cargos for diagnosis, intra-operative surgery and therapy of PCa. Enhanced binding affinity to PSMA protein and tagging of radioisotopes for treatment of metastatic castration resistant prostate cancer (mCRPC) for which no cure is known.

FIG. 1b illustrates molecular modelling method for the inhibitors, according to an embodiment as disclosed herein.

FIG. 1c illustrates chemical synthesis of designed inhibitor (L-glutamic acid based amino acetamide inhibitors), according to an embodiment as disclosed herein.

FIG. 1d illustrates design of bioconjugate handle in the linker for attaching cargos such as near-infrared fluorescent agents or anticancer drugs, according to an embodiment as disclosed herein. The peptide spacer as can be observed in this figure comprises of 2 phenylalanine residues, and the fluorescent molecule herewith attached to the peptide spacer is rhodamine.

FIG. 1e illustrates design of hydrophobic peptidic spacer for enhanced binding affinity to PSMA protein along with chelating moieties for tagging nuclear radioisotopes or nanoparticles that can be used for imaging and therapy, according to an embodiment as disclosed herein. The peptide spacer as can be observed in this figure comprises of 3 phenylalanine residues.

In an embodiment in FIG. 1b, FIG. 1c, FIG. 1d and FIG. 1e, the molecules or inhibitors have been designed after carrying out extensive in silico molecular docking and quantitative structure activity relationship (QSAR) studies. The effect of substituents and structural activity relationship of the newly designed molecules with the binding cavity of the PSMA protein of interest has been thoroughly studied by modeling studies. After the rational design of the molecules or inhibitors or ligands, using computational methods a new, mild and simple chemical synthetic strategy is developed to synthesize a small library of new PSMA inhibitors. All the inhibitors or ligands or molecules were characterized by various spectroscopic techniques for structural confirmation. Finally, inhibitors which had shown the most promising theoretical binding affinity to PSMA protein labeled as AAPT were carried forward for in vitro and in vivo biological evaluations. The high binding affinity inhibitor AAPT is also conjugated with fluorescent tag such as rhodamine B through a peptidic spacer and the resultant fluorescent conjugate was evaluated for its uptake in malignant cell lines expressing the targeted protein PSMA of interest. The binding affinity constant KD of the fluorescent peptide conjugate to PSMA protein is determined to be in nano-molar concentration. In vitro evaluation studies have confirmed the selectivity and specificity of the molecules to bind to cancer cell lines over-expressing PSMA protein. The work now awaits in vivo evaluation in small animals such as mice, rat or guinea pigs which will be carried out in near future by tagging the inhibitors via chelating linkers to radioisotopes such as ^{99m}Tc, ⁶⁸Ga, ¹⁸F for diagnostic and therapeutic (¹⁷⁷Lu) applications.

FIG. 2 illustrates design of lead aminoacetamide peptidomimetic 1 for PSMA enzyme inhibition, according to an embodiment as disclosed herein.

In an embodiment in FIG. 2, the initial design of the small molecule inhibitor is based on the understanding of the catalytic active site of metalloprotease enzyme, PSMA. The site of cleavage of the substrates has been shown for both NAAG and folyl γ-glutamate by PSMA enzyme.

Keeping the architecture of endogenous PSMA substrates e.g. NAAG as well as folyl-γ-Glu constant, at P1′ pocket, an extra carbon has been inserted after the scission of amide bond, in the newly designed inhibitors, to enhance the number of new interactions in the binding pocket of PSMA. In our initial model, P1 pocket contains another L-glutamic acid residue which is strategically attached with an extra carbon atom to form the aminoacetamide moiety. The carbonyl oxygen of amino-acetamide moiety coordinates with the Zn atom in the active site of PSMA.

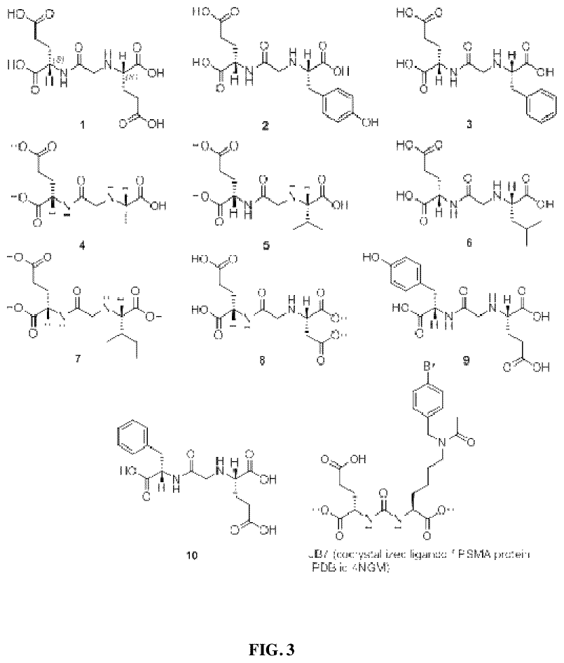

FIG. 3 illustrates design of library of peptidomimetics based on amino acetamide scaffold, according to an embodiment as disclosed herein.

In an embodiment in FIG. 3, further a small library of L-glutamic acid based amino acetamide inhibitors 1-10 which closely resembles NAAG as well as folyl-γ-Glu has been designed to examine the structural and functional requirements necessary for binding in the active site of the PSMA protein.

Table 1 shows results of docking study are reported in the form of docking score which depends on various parameters like hydrogen bonding, lipophilic and π-π interactions. Molecular docking study used in the present disclosure to examine ligand-protein interactions of designed ligands responsible for inhibition of PSMA enzyme. In the present disclosure, various amino-acetamide based ligands were designed and their protein binding affinity were analysed through molecular docking. After validation using standard ligand JB7, same docking protocol was applied for designed ligands 1-10 and the docking scores of ligands 1-10 and JB7 are shown in table 1. Surflex Dock module of Sybyl X 2.1.1. program was utilized to find the binding conformations of JB7 and newly designed amino-acetamide derivatives 1-10 at the active site of GCPII protein. It is well documented that the active site of the GCPII protein contains hydrophobic (S1 pocket) and hydrophilic pocket (S1′ pocket) and the interactions at S1′ site is believed to be more critical for better binding affinity. Literature report suggest that JB7 interact with several amino acids residues such as Tyr 700, Arg 210, Lys 699, Asn 257, Gly 518, Tyr 552, Glu 424, Asn 519, Arg 536 and Arg 534 through hydrogen bonds that are critical for better binding of ligand with the protein. The aforementioned aminoacid interactions should be taken into consideration while performing docking studies of newly designed ligands 1-10 with GCPII protein.

TABLE 1
Molecular docking scores of ligands 1-10
and JB7 with GCPII protein (PDB 4NGM)

| Rank | Ligand | Docking score |
|---|---|---|
| 1 | JB7 | 16.38 |
| 2 | 1 | 13.96 |
| 3 | 3 | 13.63 |
| 4 | 2 | 12.51 |
| 5 | 6 | 12.01 |
| 6 | 9 | 11.99 |
| 7 | 8 | 11.45 |
| 8 | 4 | 11.35 |
| 9 | 7 | 10.94 |
| 10 | 10 | 10.79 |
| 11 | 5 | 10.67 |

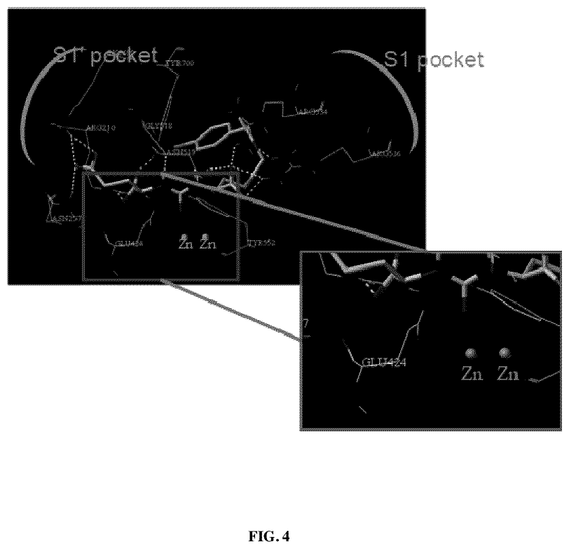

FIG. 4 illustrates molecular docking study of JB7 with GCPII protein (PDB id-4NGM), according to an embodiment as disclosed herein.

Table 2 shows a correlation between the amino acid residues of S1 and S1′ pockets present in PSMA protein and the newly designed amino-acetamide derivatives 1-10 interacting through hydrogen bonds. During the re-docking study of JB7 with the protein, similar amino acids interactions were observed as reported for JB7 with native protein (PDB 4NGM). In addition, an extra hydrogen bonding interaction with Lys 699 and Arg 536 residues were also observed. Moreover, in Surflex dock module of SYBYL, hydrogen bond distance more than 3 Å length are considered as weak and are not visible during docking study. As a result, hydrogen bonding interaction of JB7 with aminoacid residue Glu 424 of the protein was not observed which is of the order of 3.1 Å in length. After successful validation of docking procedure of JB7 with GCPII, similar protocol was applied for studying the docking interactions of newly designed amino-acetamide derivatives 1-10 with GCPII.

TABLE 2
Hydrogen bonding interactions between GCPII protein and ligands 1-10 along with bond distance of interaction in Å.

| | | JB7 H-bonding | JB7 Post or | | | | | | | | | | |
|---|---|---|---|---|---|---|---|---|---|---|---|---|---|
| | | interaction | re docking | | | | | | | | | | |
| | Amino | with GCPD in | interactions | | | | | | | | | | |
| | acid | Å from | with GCPII | H-bonding interactions of ligands 1-10 with GCPII protein in Å | | | | | | | | | |
| Site | residue | PDB 4NGM | in Å | 1 | 3 | 2 | 6 | 9 | 8 | 4 | 7 | 10 | 5 |
| S1′ site | Arg 210 | 2.8 | 2.3 | 2.72 | 1.89 | 1.87 | 1.74 | 2.03 | 2.12 | 2.34 | 2.18 | 2.03 | 1.86 |
| of GCPII | | | | 2.05 | 2.47 | | 2.41 | | | | | | 2.49 |
| (Hydrophilic | Asn 257 | 2.89 | 2.03 | 1.90 | 1.94 | | 1.89 | 1.92 | 2.72 | 2.32 | 2.00 | 1.78 | 1.93 |
| pocket) | | | | | 2.40 | | 2.70 | | 1.91 | 2.73 | 2.41 | | |
| | Lys 699 | 2.7 | 2.47, 2.06 | 1.83 | 1.87 | 2.08 | 1.98 | 2.59 | | 2.25, 2.55 | | 1.88 | 1.9 |
| | | | | | 2.52 | | | 1.79 | | | | | |
| | Tyr 552 | 2.63 | 2.65 | 1.72 | 1.89 | 2.66 | 2.33 | 2.64 | 2.27 | 1.7 | 2.35 | 2.45 | 2.16 |
| | | | | | | 1.79 | 2.54 | | | | | | |
| | Tyr 700 | 2.53 | 1.8 | 2.73 | | 1.84 | 2.2 | | 2.32 | 2.27 | | 2.69 | 2.72 |
| | | | | | | | | | 2.06 | | | | |
| | Glu 424 | 3.01 | | 2.06 | 2.04 | 2.79 | 2.67 | 2.41 | 2.1 | 1.91 | | | |
| | | | | 2.03 | 1.85 | 1.85 | 2.23 | 2.01 | | | | | |
| | Glu 425 | | | 2.01 | 2.13 | | 2 | | 2.55 | 2.55 | | | 2.13 |
| | | | | | | | | | | | | | 2.74 |
| | | | | | | | | | | | | | 2.44 |
| S1 site | Gly 51S | 3.04, 3.05 | 1.88 | 2.4 | 2.15 | | | | 1.76 | 2.44 | 1.94 | 1.9 | |
| of GCPII | | | | | | | | | | 1.85 | | | |
| (Hydrophobic | Asn 519 | 2.98 | 2.16 | 2.10 | 2.47 | | 1.99 | 1.81 | 2.04 | 2.28 | 2.08 | | |
| pocket) | | | | | 2.07 | | | | | | | | |
| | Arg 534 | 2.84 | 2.03 | 1.91 | 2.09 | | 2.21 | 2.03 | 2.03 | 1.83 | 2.04 | 2.53 | |
| | | | | 1.92 | | | | | | 2.06 | | | |
| | Arg 536 | 2.99, 3.0 | 2.32, 1.99, 1.96 | 2.21 | | | | | 2.68 | | | 2.61 | |
| | | | | 2.21 | | | | | 2.57 | | | 2.08 | |
| | | | | | | | | | 1.90 | | | 2.31 | |
| | Asp 453 | | | 2.51 | | 2.57 | | | | | | | |
| | Asp 387 | | | | | 2.56 | | | | | 2.37 | | |
| | | | | | | 2.72 | | | | | | | |
| | Ser 454 | | | | | 2.29 | | | | | 1.72 | | |
| | | | | | | | | | | | 2.30 | | |
| | | | | | | | | | | | 1.89 | | |
| | Tyr 549 | | | | | 2.34 | | 1.8 | | | 2 | | |
| | Ser 517 | | | | | 2.03 | | 2.23 | | 2.05 | | | |
| | Arg 463 | | | | | | | | 2.4 | | | | |
| Total number of | 6/6 | 6/6 | 9/7 | 10/4 | 7/6 | 11/2 | 7/4 | 8/7 | 9/6 | 4/8 | 5/5 | 9/0 | |
| hydrogen bonds | | | | | | | | | | | | | |
| at S1′/S1 | | | | | | | | | | | | | |

In an embodiment in FIG. 4, among all the newly designed amino-acetamide ligands, the docking score of ligand 1 is found to be highest followed by ligands 3 (second most active) and 2 (third most active) in the series (Table 1). The docking conformation of the most active ligand, 1, in the series show nine hydrogen bonding interactions at hydrophilic S1′ pocket with Tyr 700, Arg 210, Lys 699, Asn 257, Tyr 552, Glu 424 and Glu 425 (new hydrogen bonding interactions) residues. This fact suggested that the glutamate moiety of 1 interacts strongly with the protein active site compared to JB7. However, ligand 1 exhibits similar hydrogen bonding interaction as JB7 with hydrophobic S1 pocket (FIG. 5). Insertion of an extra methylene group after the amide bond leads to an increase in the number of hydrogen bond interactions with aminoacid residues such as Arg 210, Asn 257, Glu 424, Asn 519 and Arg 534. The carbonyl oxygen of amino acetamide ligand in 1 also interacts with the hydroxy group of Tyr 552 in S1′ pocket of PSMA with a bond length of 1.72 Å which is considerably less than the bonding interaction (2.65 Å) of urea carbonyl oxygen moiety of JB7 with OH group of Tyr 552. It is important to note that Tyr 552 is positioned near Zn atoms of GCPII protein, which is important for catalytic activity of PSMA.

FIG. 5 illustrates molecular docking study of ligand 1 designated as AAPT at GCPII active cavity (PDB id-4NGM), according to an embodiment as disclosed herein.

In an embodiment in FIG. 5, Strong interaction of 1 with Tyr 552 residue at the active site of the protein might be helpful in inhibiting the catalytic activity of GCPII. This strong hydrogen bonding interaction of 1 with GCPII could be responsible for higher binding affinity of 1 among other ligands in the series. Though ligand 1 forms more number of hydrogen bonds due to the presence of polar glutamate scaffold, the overall docking score of JB7 was found to be higher as compared to 1. This is because the efficacy of inhibitory activity of ligands not only depends on polar interactions but also on other non-polar interactions such as lipophilic and π-π stacking interactions due to the presence of benzyl group in the lysine moiety of JB7. Further, to analyze the effect of polar and non-polar substituents on the efficiency of ligands to inhibit activity of GCPII enzyme, several derivatives of amino acetamide ligands such as 2-10 have been designed and synthesized for evaluation. After glutamate ligand 1, phenylalanine (3) and tyrosine (2) derivatives were observed to have better docking score than the other designed analogs. The docking conformation of the second most active ligand (3) in series show ten hydrogen bonding interaction with the active site residues such as Arg 210, Asn 257, Lys 699, Tyr 552 and Glu 424 at the hydrophilic pocket of the enzyme. Slightly less activity of 3 as compared to 1 is due to less number of hydrogen bonding interactions (Table 2) of 3 at the hydrophobic pocket of GCPII enzyme.

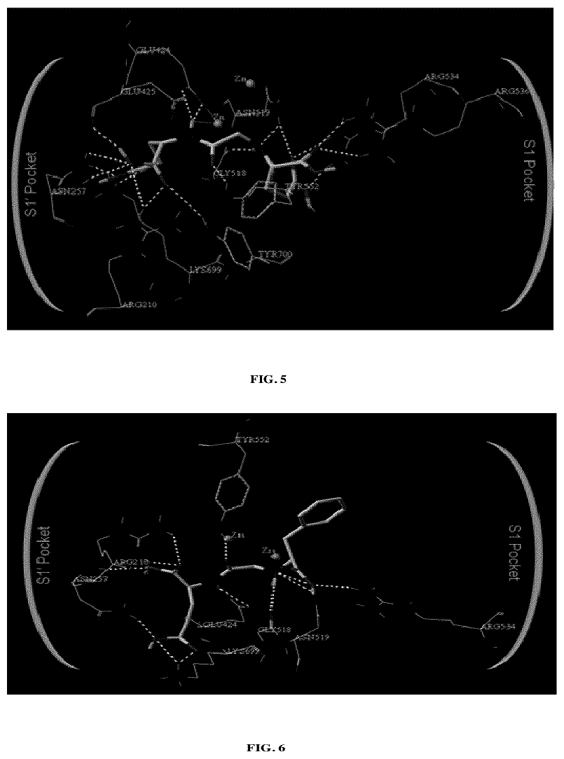

FIG. 6 illustrates computational docking of aminoacetamide inhibitor 3 in the active site of GCPII (PDB=4NGM), according to an embodiment as disclosed herein.

In an embodiment in FIG. 6, because of the presence of hydroxyl group of tyrosine moiety in 2, there is a change in the orientation of tyrosine moiety leading to change in the total number of amino acid interactions of 2 as compared to 3 with GCPII. At S1′ site (hydrophilic pocket), 3 forms seven hydrogen bonding interactions with Arg 210, Asn 257, Lys 699 and Glu 425 aminoacid residues, whereas 2 forms only two hydrogen bonding interactions with Arg 210 and Lys 699 and no interaction with Asn 257 and Glu 425 residues when compared to 3. However, 2 forms an additional H-bonding interaction with Tyr 552 along with a new interaction with Tyr 700 in the active site of GCPII in comparison to 3. Including two interactions of both 2 and 3 with Glu 424 residue, the total number interactions of 2 and 3 at hydrophilic pocket are seven and ten respectively.

FIG. 7 illustrates hydrogen bonding interactions of aminoacetamide inhibitor 2 in the active site of GCPII (PDB=4NGM) in comparison to 3, according to an embodiment as disclosed herein.

In an embodiment in FIG. 7, at S1 site (hydrophobic pocket), 3 forms four hydrogen bonding interactions with Gly 518, Asn 519 and Arg 534 which were absent in 2. Due to change in the orientation of 2, it forms six new interactions with Asp 453, Asp 387, Ser 454, Tyr 549 and Ser 517 aminoacid residues that were absent in 3. This study infers that at GCPII active site, 3 forms fourteen hydrogen bonding interactions while 2 forms only thirteen interactions. This may be the plausible reason for less docking score of ligand 2 as compared to 3. In summary, we have designed and developed amino acetamide derivatives as a new class of GCPII inhibitor through extensive molecular docking studies. Ten amino acetamide based ligands/inhibitors (1-10) have been rationally designed and through molecular docking study top three rank derivatives (1-3) were selected for detailed analysis of amino acid-ligand interactions. However, derivatives 4-10 were also studied for various interactions at the active site of GCPII without detailed interpretation of the theoretical data presented in table 2.

In an embodiment in FIG. 4, FIG. 5, FIG. 6 and FIG. 7, silico study and hydrogen bonding interactions between the designed inhibitors or ligands and different amino acids (bond length in A) present in the binding pockets of prostate specific membrane antigen (PSMA) enzyme. In the drug discovery, for prediction of binding mode of an active ligand over a protein, docking studies are usually performed. In the present disclosure, Glutamate Carboxy Peptidase II (GCPII) receptor or prostate specific membrane antigen (PSMA), complexed with urea-based inhibitor JB7 (PDB code 4NGM) was retrieved from protein data bank and docking studies were performed by surflex dock method using sybyl X2.1.1 software.

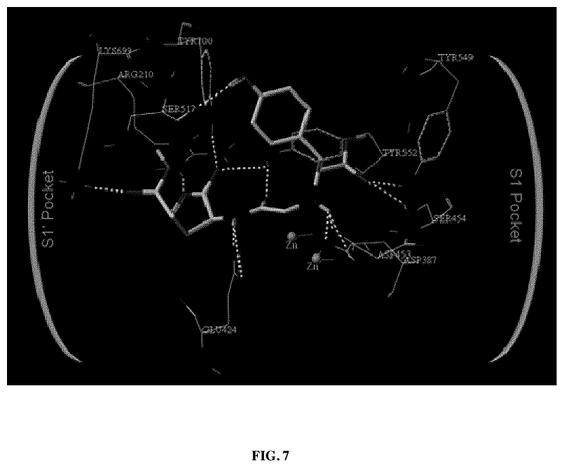

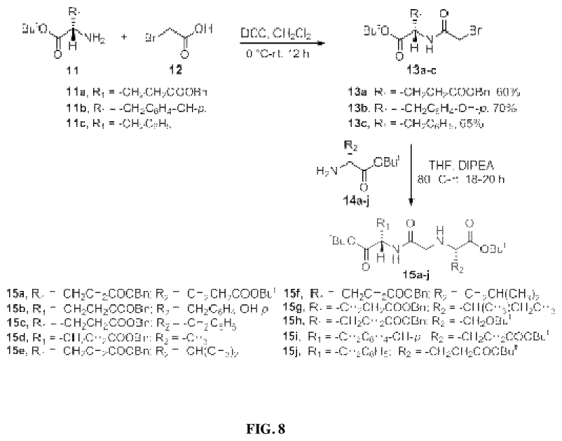

FIG. 8 illustrates synthesis of precursors 15a-j for acetamide based GCPII ligands 1-10, according to an embodiment as disclosed herein. The process for synthesis of the acetamide based GCPII inhibitors 1-3 from precursors 15a-c is provided in the examples section of the specification. The acetamide based GCPII inhibitor precursors 15a-15j synthesized by said process are illustrated in FIG. 9.

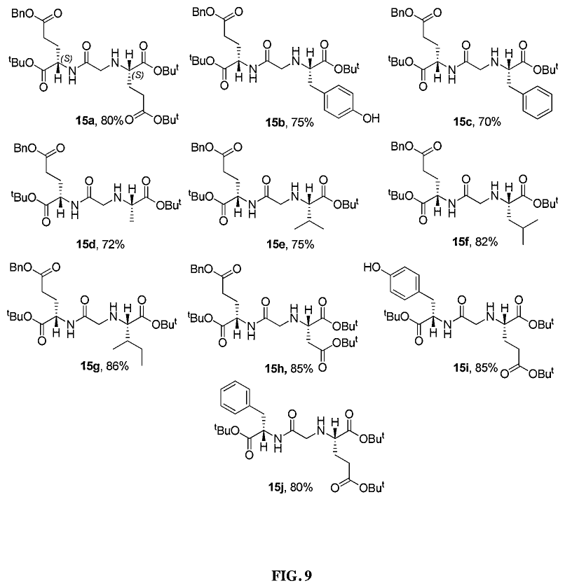

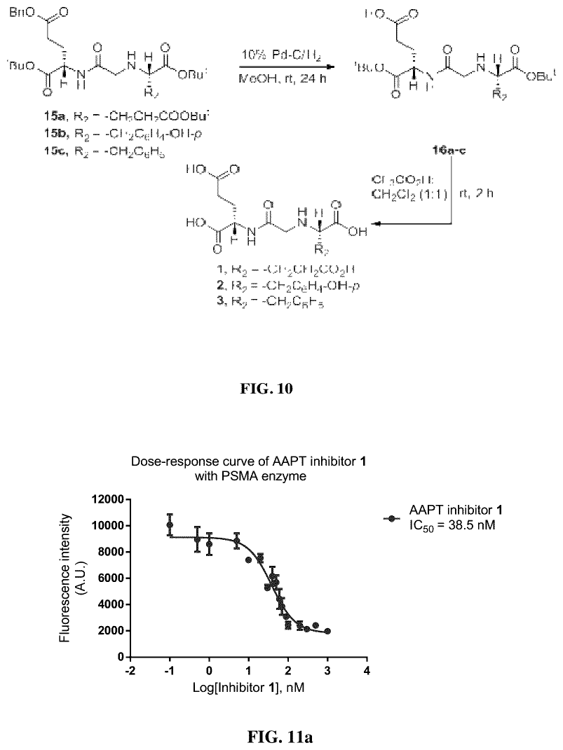

FIG. 10 illustrates synthesis of acetamide based GCPII inhibitors 1-3 from precursors 15a-c, according to an embodiment as disclosed herein. The process comprises catalytic reduction (Pd—C/H2) in the presence of methanol at room temperature for a period of 24 hours, followed by de-protection of t-butyl group on the carboxy terminus using CF3COOH in CH2Cl2 (1:1) at room temperature for 2 hours to obtain the acetamide based GCPII inhibitors 1-3.

In an embodiment in FIG. 8, FIG. 9 and FIG. 10, chemical synthetic strategy for the preparation of small molecule inhibitors and PSMA enzyme inhibition assay, chemical synthesis of inhibitors 1-3. After the completion of molecular docking study, inhibitors 1-3 were chemically synthesized by deprotection of carboxy protecting groups (benzyloxy and tertiary-butyl) from their precursors 15a-c and selected for further in vitro biological evaluation to inhibit GCPII enzyme activity. Carboxy protected acetamide precursors 15d-j of acetamide based GCPII inhibitors 4-10 were also synthesized and completely characterized for future studies if required. As per molecular docking studies GCPII inhibitors 4-10 (table 2) had poor docking scores when compared to inhibitors 1-3 and hence not synthesized chemically in the lab though their precursors 15d-j are synthesized, characterized using various spectroscopic techniques and readily available for further experimental studies. In vitro PSMA enzyme inhibition NAALADase assay for AAPT inhibitor 1. The new PSMA inhibitor 1, called as AAPT, was next analyzed for its ability to inhibit PSMA protein expressed on LNCaP cancer cell to hydrolyze a natural substrate N-acetylaspartylglutamate (NAAG) using fluorescence based Amplex Glutamate kit by competitive inhibition. The results of this study are presented in FIG. 11a-11c. Briefly, PSMA enzyme was extracted from membrane lysates of LNCaP cell lines after separating the soluble cytosolic PSM′ portion by ultracentrifugation at 100,000×g. The PSMA enzyme (8.3027 ng, 100 μL) free from cytosolic portion was incubated with various concentrations of the AAPT inhibitor (0.1 nM to 1000 nM, 100 μL) in the presence of NAAG (30 nM, 50 μL) for 60 min in a 24-well plate. The amount of glutamic acid released by the hydrolysis of NAAG during competitive inhibition by AAPT inhibitor was measured by incubating each well with a working solution of Amplex Red reagent (100 uM, 50 μL) for 30 min at 37° C. The fluorescence emission from each well, proportional to the amount of released glutamic acid, was measured using Synergy H1 multimode plate reader (BioTek Instruments, Inc., Winooski, VT, USA) at an excitation and emission wavelength of 530 nm and 590 nm respectively.

Further dose vs response curves were obtained from a plot of semi-log[conc] vs intensity of fluorescence emission and IC50 (concentration at which 50% of the enzymatic activity is inhibited) was calculated for AAPT inhibitor against PSMA enzyme using GraphPad Prism, version 7.02 for Windows (GraphPad Software, San Diego, CA). In the first trial, the enzyme inhibition analysis was performed by incubating PSMA enzyme with increasing concentrations of inhibitor, AAPT. The half maximal inhibitory concentration (IC50) of the inhibitor, AAPT, was determined to be 38.5 or 95 or 78 nM (FIG. 11a-11c).

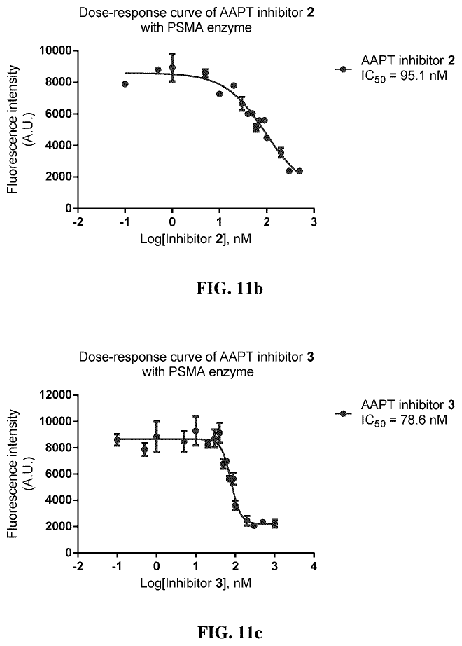

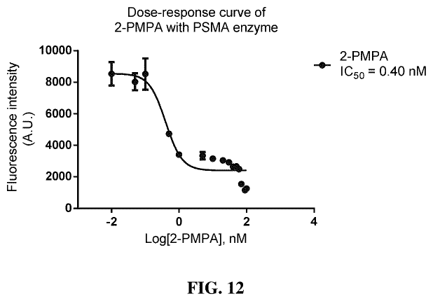

FIG. 12 illustrates dose-response curve of standard inhibitor, 2-PMPA, against PSMA protein isolated from prostate cancer cells, LNCaP; Error bars represent SD (n=3). For this purpose, GCPII enzyme inhibition assay was performed with a standard PSMA inhibitor, 2-(phosphonomethyl) pentanedioic acid (PMPA) whose IC50 is reported to be 0.28 nM and experimentally we have observed a value of IC50=0.40 nM (FIG. 12) validating our assay for inhibiting GCPII with PSMA inhibitors 1-3.

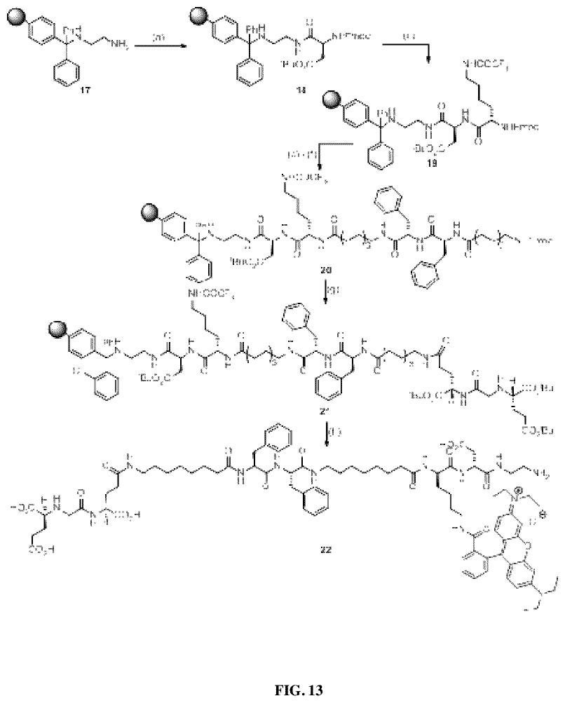

FIG. 13 illustrates solid phase synthetic strategy for preparation of AAPT rhodamine B conjugate 22 AAPT-C17201 reagents and conditions, according to an embodiment as disclosed herein.

In an embodiment in FIG. 13, Strategy for introduction of fluorescent imaging moiety or cargo (anticancer drugs) using differentially protected lysine amino acid in a continuous process through solid phase peptide synthesis (SPPS). Solid phase peptide synthesis of AAPT rhodamine B conjugate 22. 1,2-diaminoethanetrityl resin (0.050 g, 0.0525 mmol) was swelled initially with CH2Cl2 (5 mL) for 30 minutes by bubbling nitrogen and after draining CH2Cl2, the resin is swelled once again with DMF (5 mL) thrice for 15 minutes each. Fmoc-Asp(OtBu)-OH (0.054 g, 0.1312 mmol), PyBOP (0.068 g, 0.1312 mmol) and DIPEA (0.091 mL, 0.525 mmol) in DMF (0.5 mL) was added to the peptide vessel and the coupling reaction was continued for 6 h. The resin was washed with DMF (3.0 mL×3) followed by isopropanol (3.0 mL×3). The completion of reaction was confirmed by performing the Kaiser test. A solution of 20% piperidine in DMF (4 mL) was added to the peptide vessel and the resin beads were bubbled for 10 minutes. The procedure was repeated twice (3 mL×2) to ensure complete deprotection of Fmoc protecting group from the coupled amino acid. The resin beads were washed with DMF (3.0 mL×3) and isopropanol (3.0 mL×3) and the formation of free amine was confirmed by performing the Kaiser test. Consecutively, Fmoc-Lys(Tfa)-OH, Fmoc-8-aminocaprylic acid, Fmoc-Phe-OH, Fmoc-Phe-OH and Fmoc-8-aminocaprylic acid were attached to the growing peptide chain in sequence as mentioned before. After deprotection of Fmoc group from the last amino acid, Fmoc-8-aminocaprylic acid, tris-tert butyl protected AAPT ligand 16a (0.040 g, 0.079 mmol), PyBOP (0.068 g, 0.1312 mmol) and DIPEA (0.091 mL, 0.525 mmol) in DMF (0.5 mL) was added to the resin beads and mixed for 6 h. The completion of coupling reaction was confirmed by performing the Kaiser test. Finally, the trifluoroacetyl (Tfa) protecting group of lysine amino acid was cleaved with 2M aqueous piperidine (10 mL) at room temperature for 6-12 h (depending on completion of the reaction) and the deprotection of Tfa group was confirmed by the Kaiser test. Rhodamine B dye (0.038 g, 0.079 mmol), PyBOP (0.068 g, 0.1312 mmol) and DIPEA (0.091 mL, 0.525 mmol) in DMF (0.5 mL) was added to the peptide vessel and reacted for 6 h at room temperature. The completion of the coupling reaction was confirmed by performing the Kaiser test. A mixture of 9.5 mL trifluoroacetic acid (TFA), 0.25 mL triisopropylsilane (TIPS), and 0.25 mL H2O was prepared in a 15 mL centrifuge tube, and 5 mL of this cocktail solution was added to the resin beads and nitrogen gas was bubbled through the solution for 30 minutes. The cocktail with cleaved rhodamine peptide conjugate in peptide vessel was collected to a round bottom flask (25 mL). The resin beads were treated again with the cocktail solution twice (2.5 mL×2) for 15 minutes each and the mother liquor was collected in the same round bottom flask (25 mL). The pooled cocktail mixture with cleaved peptide conjugate 22 was transferred to a 15 mL centrifuge tube, fitted with a septum and concentrated under reduced pressure to obtain a viscous liquid. Ice cold ether (5 mL) was added to the concentrated viscous mixture to precipitate rhodamine conjugate 22 as a bright red solid and the solid was washed thrice with ice cold ether (5 mL×3). The crude product 22 was purified by RP-HPLC using pentafluorophenyl preparative column (5 μm, 10 mm×150 mm) [λ=555 nm; solvent gradient 1% B to 70% B in 25 min, 80% B wash 15 min; A=0.1% TFA, pH=2; B=acetonitrile (ACN)]. Acetonitrile was removed reduced pressure from HPLC fractions, and the pure fractions were freeze-dried to yield peptide rhodamine conjugate 22 as pink solid. The purity of rhodamine conjugate 22 was confirmed by analytical RP-HPLC and the molecular weight is determined by HRMS (+ ESI) calcd for [M-Cl]+(C86H118N13O18)+: 1620.8718 found 1620.8727.

In an embodiment in FIG. 13, Solid phase synthetic strategy for preparation of AAPT rhodamine B conjugate 22, AAPT-C17201, Reagents and conditions (a) Fmoc-Asp(OtBu)-OH, PyBOP, DIPEA, DMF, 6 h (b) (i) 20% Piperidine in DMF, rt, 30 min (ii) Fmoc-Lys(Tfa)-OH, PyBOP, DIPEA, DMF, 6 h (c) (i) 20% Piperidine in DMF, rt, 30 min (ii) Fmoc-8-aminocaprylic acid, PyBOP, DIPEA, DMF, 6 h (d) (i) 20% Piperidine in DMF, rt, 30 min (ii) Fmoc-Phe-OH, PyBOP, DIPEA, DMF, 6 h (e) (i) 20% Piperidine in DMF, rt, 30 min (ii) Fmoc-Phe-OH, PyBOP, DIPEA, DMF, 6 h (f) (i) 20% Piperidine in DMF, rt, 30 min (ii) Fmoc-8-aminocaprylic acid, PyBOP, DIPEA, DMF, 6 h (g) (i) 20% Piperidine in DMF, rt, 30 min (ii) 16a, PyBOP, DIPEA, DMF, 6 h (h) (i) 2M Piperidine in water, rt, 6-12 h (ii) Rhodamine B, PyBOP, DIPEA, DMF, 6 h (iii) TFA/TIS/H2O (95.0:2.5:2.5) (1×5 mL, 30 min; 2×2.5 mL, 15 min each) (iv) Evaporate TFA (v) Precipitate in ice cold diethylether.

FIG. 14 illustrates laser scanning confocal microscopy uptake study of PSMA targeted AAPT-C17201 fluorescent conjugate 22 in prostate cancer cell LNCaP at various concentrations, according to an embodiment as disclosed herein.

In an embodiment in FIG. 14, Uptake study of ligand conjugated fluorescent conjugate 22, AAPT-C17201, in prostate cancer cells, LNCaP, using laser scanning confocal microscopy. The selective uptake of the newly synthesized PSMA targeted near-infra red fluorescent conjugate, AAPT-C17201, was evaluated by studying the ability of AAPT inhibitor or ligand to deliver diagnostics or fluorescent cargos to PSMA protein expressing cancer cell lines such as LNCaP cells. The fluorescent conjugate, AAPT-C17201, was synthesized by tethering a high affinity AAPT inhibitor via a peptidic spacer to Rhodamine B using standard solid phase peptide synthesis methodology. The purified PSMA targeted ligand conjugate, AAPT-C17201, was evaluated in prostate cancer cells (LNCaP) expressing PSMA protein using laser scanning confocal microscopy for wide range of low nanomolar concentrations-5, 10, 25 nM. Laser scanning confocal microscopy uptake study of PSMA targeted AAPT-C17201 fluorescent conjugate 22 in prostate cancer cells, LNCaP, at various concentrations (i) 5 nM, (ii) 10 nM, (iii) 25 nM for 1 h incubation and (iv-vi) differential interference contrast (DIC) images of PCa cells.

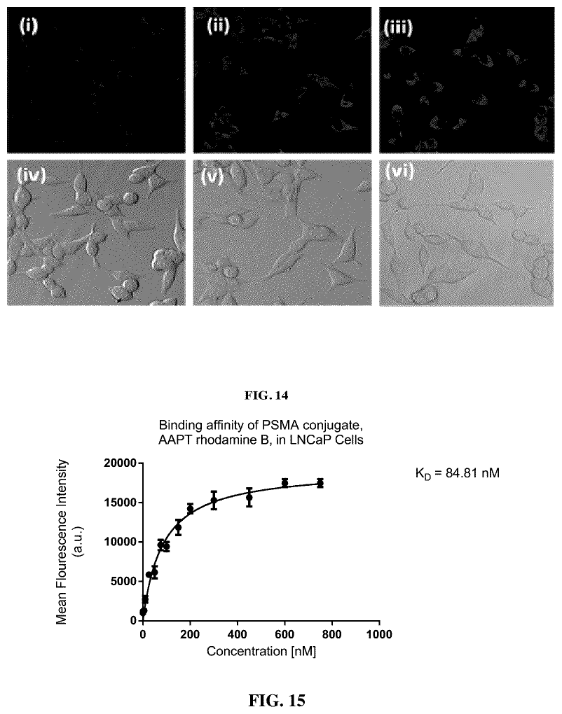

FIG. 15 illustrates binding affinity constant KD, determination of fluorescent conjugate 22 AAPT-C17201 in prostate cancer cell LNCaP SD (n=3), according to an embodiment as disclosed herein.

In an embodiment in FIG. 15, Evaluation of binding affinity of PSMA targeted fluorescent conjugate 22, AAPT-C17201, using flow cytometry analysis. The binding affinity constant of PSMA targeted fluorescent conjugate, AAPT-C17201, to target PSMA was determined in vitro in LNCaP cells by measuring the mean fluorescence intensity per cell for different concentrations of the fluorescent conjugate in triplicate using Fluorescence Activated Cell Sorting (FACS) study. A hyperbolic curve of different concentrations of fluorescent conjugates 22 or 25 against the mean fluorescence intensity in the PSMA+ LNCaP cells yields a dissociation constant (KD) of 85 nM or 130 nM, respectively. The high dissociation constant value (˜85 nM) gives us undisputable evidence for the high affinity of AAPT-C17201 fluorescent conjugate to PSMA protein and its perfect fit to the 20 Å protein tunnel present in PSMA.

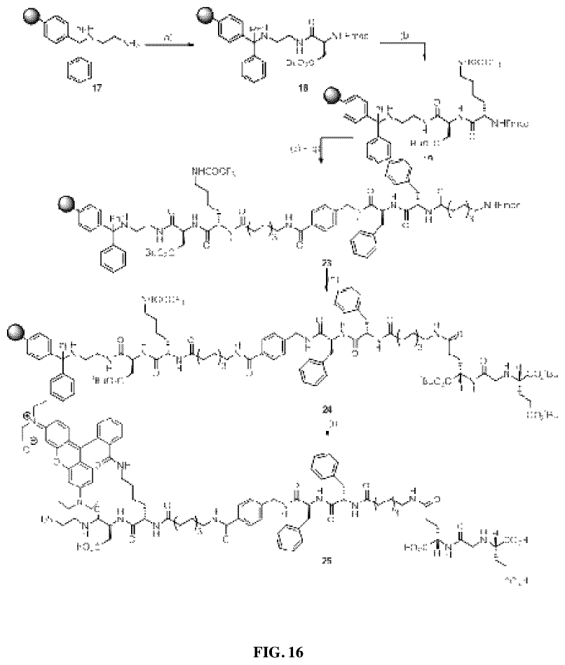

FIG. 16 illustrates solid phase synthetic strategy for preparation of AAPT rhodamine B conjugate 25 reagents and conditions, according to an embodiment as disclosed herein.

In an embodiment in FIG. 16, Solid phase peptide synthesis of AAPT rhodamine B conjugate 25. The arene rhodamine B conjugate 25 is synthesized using SPPS by introduction of 4-carboxylic acid benzylamine in the peptide spacer to enhance the binding affinity to PSMA protein. 1,2-diaminoethanetrityl resin (0.050 g, 0.0525 mmol) was swelled initially with CH2Cl2 (5 mL) for 30 minutes by bubbling nitrogen and after draining CH2Cl2, the resin is swelled once again with DMF (5 mL) thrice for 15 minutes each. Fmoc-Asp(OtBu)-OH (0.054 g, 0.1312 mmol), PyBOP (0.068 g, 0.1312 mmol) and DIPEA (0.091 mL, 0.525 mmol) in DMF (0.5 mL) was added to the peptide vessel and the coupling reaction was continued for 6 h. The resin was washed with DMF (3.0 mL×3) followed by isopropanol (3.0 mL×3). The completion of reaction was confirmed by performing the Kaiser test. A solution of 20% piperidine in DMF (4 mL) was added to the peptide vessel and the resin beads were bubbled for 10 minutes. The procedure was repeated twice (3 mL×2) to ensure complete deprotection of Fmoc protecting group from the coupled amino acid. The resin beads were washed with DMF (3.0 mL×3) and isopropanol (3.0 mL×3) and the formation of free amine was confirmed by performing the Kaiser test. Consecutively, Fmoc-Lys(Tfa)-OH, Fmoc-8-aminocaprylic acid, Fmoc benzylamine-4-carboxylic acid, Fmoc-Phe-OH, Fmoc-Phe-OH and Fmoc-8-aminocaprylic acid were attached to the growing peptide chain in sequence as mentioned before. After deprotection of Fmoc group from the last amino acid, Fmoc-8-aminocaprylic acid, tris-tert butyl protected AAPT ligand 16a (0.040 g, 0.079 mmol), PyBOP (0.068 g, 0.1312 mmol) and DIPEA (0.091 mL, 0.525 mmol) in DMF (0.5 mL) was added to the resin beads and mixed for 6 h. The completion of coupling reaction was confirmed by performing the Kaiser test. Finally, the trifluoroacetyl (Tfa) protecting group of lysine amino acid was cleaved with 2M aqueous piperidine (10 mL) at room temperature for 6-12 h (depending on completion of the reaction) and the deprotection of Tfa group was confirmed by the Kaiser test. Rhodamine B dye (0.038 g, 0.079 mmol), PyBOP (0.068 g, 0.1312 mmol) and DIPEA (0.091 mL, 0.525 mmol) in DMF (0.5 mL) was added to the peptide vessel and reacted for 6 h at room temperature. The completion of the coupling reaction was confirmed by performing the Kaiser test. A mixture of 9.5 mL trifluoroacetic acid (TFA), 0.25 mL triisopropylsilane (TIPS), and 0.25 mL H2O was prepared in a 15 mL centrifuge tube, and 5 mL of this cocktail solution was added to the resin beads and nitrogen gas was bubbled through the solution for 30 minutes. The cocktail with cleaved rhodamine peptide conjugate in peptide vessel was collected to a round bottom flask (25 mL). The resin beads were treated again with the cocktail solution twice (2.5 mL×2) for 15 minutes each and the mother liquor was collected in the same round bottom flask (25 mL). The pooled cocktail mixture with cleaved peptide arene rhodamine B conjugate 25 was transferred to a 15 mL centrifuge tube, fitted with a septum and concentrated under reduced pressure to obtain a viscous liquid. Ice cold ether (5 mL) was added to the concentrated viscous mixture to precipitate arene rhodamine B conjugate 25 as a bright red solid and the solid was washed thrice with ice cold ether (5 mL×3). The crude product 25 was purified by RP-HPLC using pentafluorophenyl preparative column (5 μm, 10 mm×150 mm) [λ=555 nm; solvent gradient 1% B to 70% B in 25 min, 80% B wash 15 min; A=0.1% TFA, pH=2; B=acetonitrile (ACN)]. Acetonitrile was removed reduced pressure from HPLC fractions, and the pure fractions were freeze-dried to yield peptide arene rhodamine B conjugate 25 as pink solid. The purity of arene rhodamine B conjugate 25 was confirmed by analytical RP-HPLC and the molecular weight is determined by HRMS (+ ESI) calcd for [M-Cl]+(C94H125N14019)+: 1753.9245 found 1753.9239.

In an embodiment in FIG. 16, Solid phase synthetic strategy for preparation of AAPT rhodamine B conjugate 25, Reagents and conditions (a) Fmoc-Asp(OtBu)-OH, PyBOP, DIPEA, DMF, 6 h (b) (i) 20% Piperidine in DMF, rt, 30 min (ii) Fmoc-Lys(Tfa)-OH, PyBOP, DIPEA, DMF, 6 h (c) (i) 20% Piperidine in DMF, rt, 30 min (ii) Fmoc-8-aminocaprylic acid, PyBOP, DIPEA, DMF, 6 h (d) (i) 20% Piperidine in DMF, rt, 30 min (ii) Fmoc benzylamine-4-carboxylic acid, PyBOP, DIPEA, DMF, 6 h (e) (i) 20% Piperidine in DMF, rt, 30 min (ii) Fmoc-Phe-OH, PyBOP, DIPEA, DMF, 6 h (f) (i) 20% Piperidine in DMF, rt, 30 min (ii) Fmoc-Phe-OH, PyBOP, DIPEA, DMF, 6 h (g) (i) 20% Piperidine in DMF, rt, 30 min (ii) Fmoc-8-aminocaprylic acid, PyBOP, DIPEA, DMF, 6 h (h) (i) 20% Piperidine in DMF, rt, 30 min (ii) 16a, PyBOP, DIPEA, DMF, 6 h (i) (i) 2M Piperidine in water, rt, 6-12 h (ii) Rhodamine B, PyBOP, DIPEA, DMF, 6 h (iii) TFA/TIS/H2O (95.0:2.5:2.5) (1×5 mL, 30 min 2×2.5 mL, 15 min each) (iv) Evaporate TFA (v) Precipitate in ice cold diethylether.

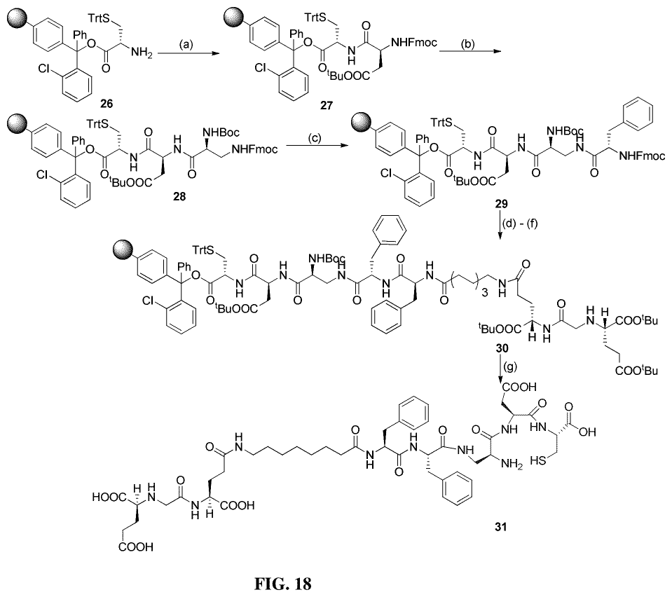

FIG. 18 illustrates synthesis of AAPT ligand conjugated chelating linker 31 reagents and conditions, according to an embodiment as disclosed herein.

In an embodiment in FIG. 18, Solid phase synthesis of AAPT ligand conjugated chelating linker 31.H-Cys-2-ClTrt resin (0.100 g, 0.09 mmol) was swelled in CH2Cl2 (5 mL) for 30 minutes by bubbling nitrogen and after draining CH2Cl2, the resin is swelled once again with DMF (5 mL×3) thrice for 15 minutes each. Fmoc-Asp(OtBu)-OH (93 mg, 0.225 mmol), PyBOP (116 mg, 0.225 mmol) and DIPEA (0.16 mL, 0.90 mmol) in DMF (0.5 mL) was added to the peptide vessel and the coupling reaction was continued for 6 h. The resin was washed with DMF (3.0 mL×3) followed by isopropanol (3.0 mL×3). The completion of reaction was confirmed by performing the Kaiser test. A solution of 20% piperidine in DMF (4 mL) was added to the peptide vessel and the resin beads were bubbled for 10 minutes. The procedure was repeated twice (3 mL×2) to ensure complete deprotection of Fmoc protecting group from the coupled amino acid. The resin beads were washed with DMF (3.0 mL×3) and isopropanol (3.0 mL×3) and the formation of free amine was confirmed by performing the Kaiser test. Consecutively, Boc-Dap(Fmoc)-OH, Fmoc-Phe-OH, Fmoc-Phe-OH and Fmoc-8-aminocaprylic acid were attached to the growing peptide chain in sequence as mentioned before. After deprotection of NHFmoc group from the last amino acid, Fmoc-8-aminocaprylic acid, tris-tertbutyl protected compound 16a (68 mg, 0.135 mmol), PyBOP (116 mg, 0.225 mmol) and DIPEA (0.16 mL, 0.90 mmol) in DMF (0.5 mL) were added to the vessel and mixed for 6 h. The completion of reaction was confirmed by the Kaiser test.

In an embodiment in FIG. 18, A mixture of 9.25 mL trifluoroacetic acid (TFA), 0.25 mL ethane dithiol, 0.25 mL triisopropylsilane (TIPS), and 0.25 mL H2O was prepared in a 15 mL centrifuge tube, and 5 mL of this cocktail solution was added to the resin beads and nitrogen gas was bubbled through the solution for 30 minutes. The cocktail with cleaved ligand targeted cysteine chelating linker 31 in peptide vessel was collected to a round bottom flask (25 mL). The resin beads were treated again with the cocktail solution twice (2.5 mL×2) for 15 minutes each and the mother liquor was collected in the same round bottom flask (25 mL). The pooled cocktail mixture with cleaved peptide conjugate 31 was transferred to a 15 mL centrifuge tube, fitted with a septum and concentrated under reduced pressure to obtain a viscous liquid. Ice cold ether (5 mL) was added to the concentrated viscous mixture to precipitate ligand targeted cysteine chelating linker 31 as a white solid and the solid was washed thrice with ice cold ether (5 mL×3). The crude product 31 was purified by RP-HPLC using pentafluorophenyl preparative column (5 μm, 10 mm×150 mm) [λ=254 nm; solvent gradient 0% B to 100% B in 45 min, 100% B wash 5 min; A=0.1% TFA, pH=2; B=0.1% TFA in acetonitrile (ACN)]. Acetonitrile was removed under reduced pressure from HPLC fractions, and the pure fractions were freeze-dried to yield ligand targeted cysteine chelating linker 31 as white solid. The purity of 31 was confirmed by analytical RP-HPLC and ESI-HRMS (m/z): [M+H]+ calcd. for C48H67N9017S, 1073.4463; found, 1073.4480. UV/vis: λmax=254 nm. Synthesis of AAPT ligand conjugated chelating linker 31; Reagents and conditions (a) Fmoc-Asp(OtBu)-OH, PyBOP, DIPEA, DMF, 6 h (b) (i) 20% piperidine/DMF, rt, 30 min (ii) Fmoc-diaminopropionic (DAP) acid, PyBOP, DIPEA, DMF, 6 h (c) (i) 20% Piperidine/DMF, rt, 30 min (ii) Fmoc-Phe-OH, PyBOP, DIPEA, DMF, 6 h (d) (i) 20% Piperidine/DMF, rt, 30 min (ii) Fmoc-Phe-OH, PyBOP, DIPEA, DMF, 6 h (e) (i) 20% Piperidine/DMF, rt, 30 min (ii) Fmoc-8-aminooctanoic (EAO) acid, PyBOP, DIPEA, DMF, 6 h (f) (i) 20% Piperidine/DMF, rt, 30 min (ii) 16a, PyBOP, DIPEA, DMF, 6 h (g) TFA/H2O/TIPS/EDT (92.5:2.5:2.5:2.5) (1×5 mL, 30 min 2×2.5 mL, 15 min) (iv) Evaporate TFA (v) Precipitate in ice cold diethylether.

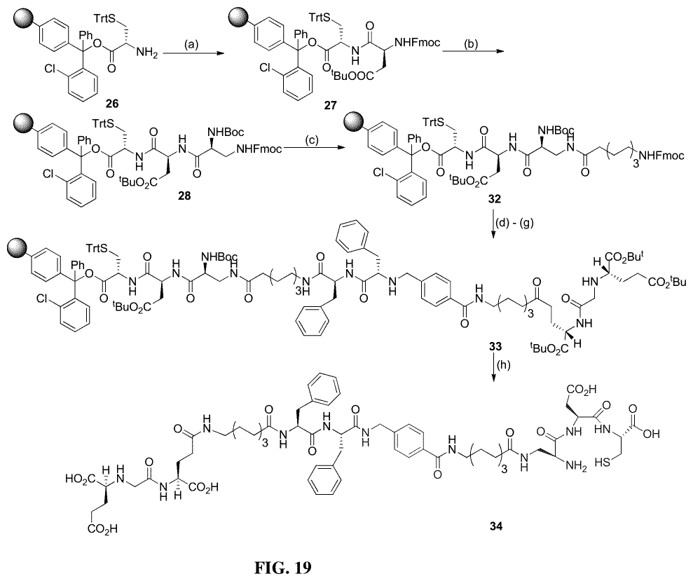

FIG. 19 illustrates synthesis of AAPT ligand conjugated arene chelating linker 34 reagents and conditions, according to an embodiment as disclosed herein.

In an embodiment in FIG. 19, Solid phase synthetic procedure for preparation of AAPT ligand conjugated arene chelating linker 34. H-Cys-2-ClTrt resin (0.100 g, 0.09 mmol) was swelled in CH2Cl2 (5 mL) for 30 minutes by bubbling nitrogen and after draining CH2Cl2, the resin is swelled once again with DMF (5 mL×3) thrice for 15 minutes each. Fmoc-Asp(OtBu)-OH (93 mg, 0.225 mmol), PyBOP (116 mg, 0.225 mmol) and DIPEA (0.16 mL, 0.90 mmol) in DMF (0.5 mL) was added to the peptide vessel and the coupling reaction was continued for 6 h. The resin was washed with DMF (3.0 mL×3) followed by isopropanol (3.0 mL×3). The completion of reaction was confirmed by performing the Kaiser test. A solution of 20% piperidine in DMF (4 mL) was added to the peptide vessel and the resin beads were bubbled for 10 minutes. The procedure was repeated twice (3 mL×2) to ensure complete deprotection of Fmoc protecting group from the coupled amino acid. The resin beads were washed with DMF (3.0 mL×3) and isopropanol (3.0 mL×3) and the formation of free amine was confirmed by performing the Kaiser test. Consecutively, Boc-Dap(Fmoc)-OH, Fmoc-8-aminocaprylic acid, 4-(Fmoc-aminomethyl) benzoic acid Fmoc-Phe-OH, Fmoc-Phe-OH and Fmoc-8-aminocaprylic acid were attached to the growing peptide chain in sequence as mentioned before. After deprotection of NHFmoc group from the last amino acid, Fmoc-8-aminocaprylic acid, tris-tertbutyl protected compound 16a (68 mg, 0.135 mmol), PyBOP (116 mg, 0.225 mmol) and DIPEA (0.16 mL, 0.90 mmol) in DMF (0.5 mL) were added to the vessel and mixed for 6 h. The completion of reaction was confirmed by the Kaiser test.

In an embodiment in FIG. 19, A mixture of 9.25 mL trifluoroacetic acid (TFA), 0.25 mL ethane dithiol, 0.25 mL triisopropylsilane (TIPS), and 0.25 mL H2O was prepared in a 15 mL centrifuge tube, and 5 mL of this cocktail solution was added to the resin beads and nitrogen gas was bubbled through the solution for 30 minutes. The cocktail with cleaved AAPT ligand conjugated arene chelating linker 34 in peptide vessel was collected to a round bottom flask (25 mL). The resin beads were treated again with the cocktail solution twice (2.5 mL×2) for 15 minutes each and the mother liquor was collected in the same round bottom flask (25 mL). The pooled cocktail mixture with cleaved AAPT ligand conjugated arene chelating linker 34 was transferred to a 15 mL centrifuge tube, fitted with a septum and concentrated under reduced pressure to obtain a viscous liquid. Ice cold ether (5 mL) was added to the concentrated viscous mixture to precipitate arene chelating linker 34 as a white solid and the solid was washed thrice with ice cold ether (5 mL×3). The crude product 34 was purified by RP-HPLC using pentafluorophenyl preparative column (5 μm, 10 mm×150 mm) [λ=254 nm; solvent gradient 0% B to 100% B in 45 min, 100% B wash 5 min; A=0.1% TFA, pH=2; B=0.1% TFA in acetonitrile (ACN)]. Acetonitrile was removed under reduced pressure from HPLC fractions, and the pure fractions were freeze-dried to yield AAPT ligand conjugated arene chelating linker 34 as white solid. The purity of 34 was confirmed by analytical RP-HPLC and ESI-HRMS (m/z): [M+H]+ calcd. for C64H89N11019S, 1347.6057; found, 1348.6048. UV/vis: λmax=254 nm. Synthesis of AAPT ligand conjugated arene chelating linker 34; Reagents and conditions (a) Fmoc-Asp(OtBu)-OH, PyBOP, DIPEA, DMF, 6 h (b) (i) 20% Piperidine/DMF, rt, 30 min (ii) Fmoc-diaminopropionic (DAP) acid, PyBOP, DIPEA, DMF, 6 h (c) (i) 20% Piperidine/DMF, rt, 30 min (ii) Fmoc-8-aminooctanoic (EAO) acid, PyBOP, DIPEA, DMF, 6 h (d) (i) 20% Piperidine/DMF, rt, 30 min (ii) 4-(Fmoc-aminomethyl)benzoic acid, PyBOP, DIPEA, DMF, 6 h (e) 20% Piperidine/DMF, rt, 30 min (ii) Fmoc-Phe-OH, PyBOP, DIPEA, DMF, 6 h (f) (i) 20% Piperidine/DMF, rt, 30 min (ii) Fmoc-Phe-OH, PyBOP, DIPEA, DMF, 6 h (g) (i) 20% Piperidine/DMF, rt, 30 min (ii) Fmoc-8-aminooctanoic (EAO) acid, PyBOP, DIPEA, DMF, 6 h (h) (i) 20% Piperidine/DMF, rt, 30 min (ii) 16a, PyBOP, DIPEA, DMF, 6 h (h) TFA/H2O/TIPS/EDT (92.5:2.5:2.5:2.5) (1×5 mL, 30 min; 2×2.5 mL, 15 min) (iv) Evaporate TFA (v) Precipitate in ice cold diethylether.

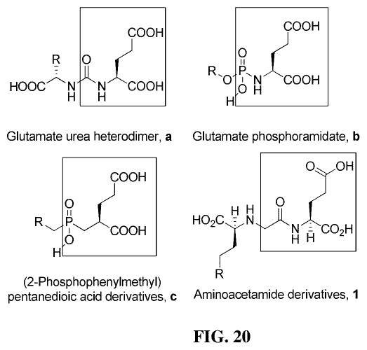

FIG. 20 illustrates structures of the PSMA scaffolds, glutamate urea heterodimers, glutamate phosphoramidates, 2-(phosphinylmethyl) pentanedioic acid and acetamide derivatives, according to an embodiment as disclosed herein.

In an embodiment in FIG. 20, based on the NAAG interaction in the binding pockets of PSMA three kinds of PSMA scaffold have been documented in literature which can inhibit the hydrolysis of NAAG, a substrate for PSMA. These molecules are named as (i) glutamate-urea heterodimers a, (ii) glutamate phosphoramidates b and (iii) 2-(phosphinylmethyl)pentanedioic acid derivatives c. Structures of the PSMA scaffolds: glutamate-urea heterodimers a, glutamate phosphoramidates b, 2-(phosphinylmethyl)pentanedioic acid c and acetamide derivatives 4.

Literature reports show that except these three classes of small molecule inhibitors, no other new small molecule inhibitors are known till date for PSMA. Research group has taken a step forward to invent a new class of ligands called as aminoacetamide 4 based PSMA inhibitors. This class of inhibitor 4 has been designed by extensive in silico studies. After optimizing docking studies with PSMA protein, we have discovered a novel, simple, mild and high yielding synthetic strategy for the preparation of 4 via retrosynthetic analysis. The newly synthesized inhibitors 4 were screened for PSMA enzyme inhibition by in vitro experiments using PSMA enzyme isolate from human prostate cancer lines. Experimental analysis shows that the newly designed inhibitors have similar inhibitory activity when compared to reported inhibitors. Fluorescent agent was tethered to 4 through a peptidic spacer to perform uptake study of the fluorescent conjugate in prostate cancer cell lines and the selectivity for PSMA+ cells was unequivocally proved.

In the present disclosure, small molecule aminoacetamide PSMA inhibitors has tremendous applications in healthcare sector to treat cancer and neurodegenerative diseases. The new inhibitors and their NIR fluorescent conjugates, radionuclear conjugates, targeted nanomaterials will be used for early diagnosis, intraoperative guided-surgery, MRI contrast imaging and treatment of prostate cancers that are resistant to hormone therapy such as metastatic castration resistant prostate cancers (mCRPC) at hospitals in India and other countries. Moreover, the inhibitors can be used to treat neurodegenerative diseases such as amyotrophic lateral sclerosis. The inhibitors or ligands and their conjugates have high potentiality to become commercial products after the preclinical evaluation.

An embodiment relates to a conjugate having a formula: D-E-F wherein D comprises AAPT ligand or a derivative thereof and having formula I

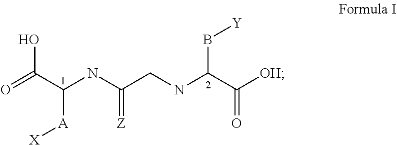

E is a spacer comprising a chain of amino acid or its derivatives comprising AA1, AA2, AA3 and/or AA4;
F is a chelating group comprises formula II

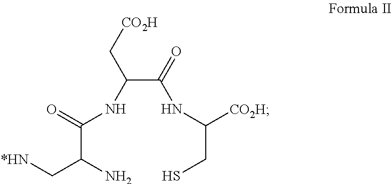

wherein A and B of formula I are independently selected from a group consisting of hydrogen, optionally substituted C₁-C₇ alkyl, and optionally substituted aryl groups; X and Y are selected from the groups comprising of —H, —OH, and —COOH groups, and Z is one of O or S groups;
wherein formula I comprises polar charged or polar uncharged amino acid or both;
wherein more than two of AA1, AA2, AA3 and/or AA4 are present;
wherein * indicates the site of attachment to the spacer;
wherein the conjugate is configured to have binding affinity≤60 nM.

In an embodiment, formula I comprises polar negatively charged amino acid.

In an embodiment, glutamic acid is present on both sides of the acetamide bond. In another embodiment, glutamic acid and aspartic acid is present on either side of the acetamide bond. In yet another embodiment, aspartic acid is present on either side of the acetamide bond.

In an embodiment, AA1 comprises trans-4-aminomethylcyclohexanecarboxylic acid or 2-naphthylalanine.

In an embodiment, AA2 comprises 2-naphthylalanine or trans-4-aminomethylcyclohexanecarboxylic acid.

In an embodiment, AA3 comprises 2-naphthylalanine or phenylalanine or NH₂—(CH₂)2-PEG_{2/3}-(CH₂)2CO₂H or NH₂—(CH₂)7-CO₂H.

In an embodiment, AA4 comprises NH₂—(CH₂)2-PEG_{2/3}-(CH₂)2CO₂H.

In an embodiment, AA4 comprises Polyethylene Glycol 3 (PEG₃).

In an embodiment, the conjugate comprises AAPT ligand conjugated arene chelating linker for delivery of ^{99m}Tc radioisotope comprises:

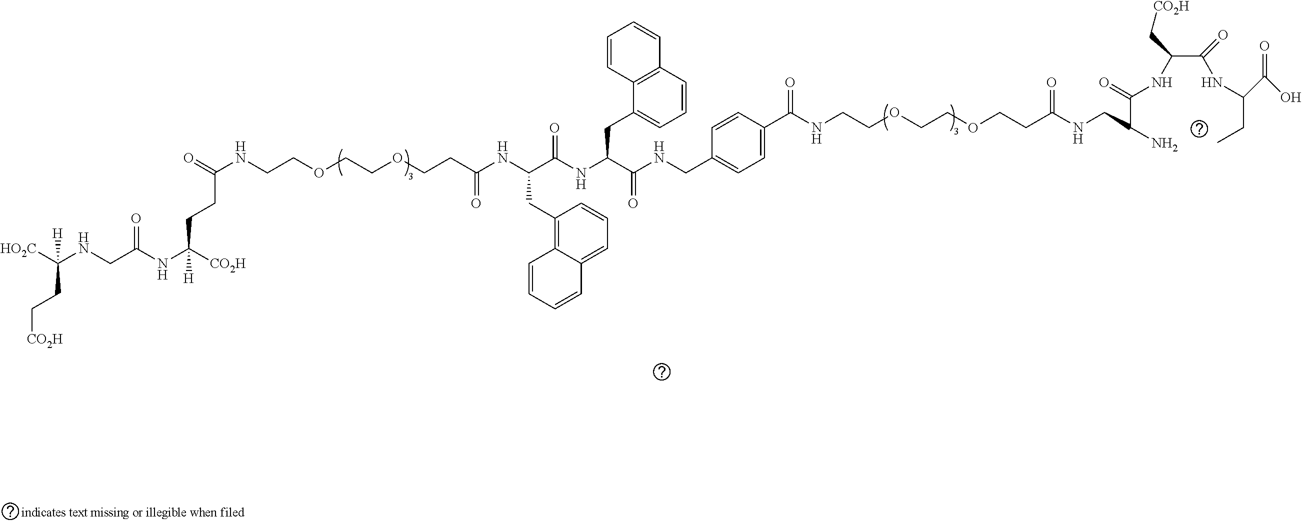

In an embodiment, in the targeting ligand of AAPT contains amino acetamide group. However, in the prior arts, in ligands e.g. DUPA ligand urea bond has been used, which has totally different properties. At first, it may seem that AAPT and DUPA only differ by —CH₂ bond which is incorrect as acetamide group is less basic than urea bond. In acetamide lone pair of nitrogen is not available for bonding. So, it has totally different dielectric properties. To even a person skilled in the art, the change in the properties dissuade to use acetamide bond or AAPT inhibitor as a candidate to make a bioconjugate for PSMA targeted cells. Further, in the AAPT-PCa bioconjugate, PEG (Polyethylene Glycol) has been used.

In an embodiment, JB7 (standard ligand) has lysine (basic amino acid), where as in the present invention glutamic acid (acidic amino acid) is present around acetamide group. In table 1 almost for all the amino acids the interaction of AAPT ligand 1 has more interaction than JB7. In an embodiment, the glutamate moiety of ligand 1 interacts strongly with the protein active site compared to JB7. However, ligand 1 exhibits similar hydrogen bonding interaction as JB7 with hydrophobic S1 pocket (FIG. 5). Insertion of an extra methylene group after the amide bond leads to an increase in the number of hydrogen bond interactions with aminoacid residues such as Arg 210, Asn 257, Glu 424, Asn 519 and Arg 534. The carbonyl oxygen of amino acetamide ligand in 1 also interacts with the hydroxy group of Tyr 552 in S1′ pocket of PSMA with a bond length of 1.72 Å which is considerably less than the bonding interaction (2.65 Å) of urea carbonyl oxygen moiety of JB7 with OH group of Tyr 552. This is because the efficacy of inhibitory activity of ligands not only depends on polar interactions but also on other non-polar interactions such as lipophilic and π-π stacking interactions due to the presence of benzyl group in the lysine moiety of JB7.

TABLE 3
Binding affinities of DUPA based
bioconjugate: From Kularatne et al. 2009
(Molecular Pharmaceutics, 6:3:790-800)

| | | K_{D} |
|---|---|---|
| Compound | Peptide Spacer (X) | (nM) |
| 3 | None | 176 |
| 7 | NH-Phe-Glu-Ala-Phe-Phe-CO | 102 |
| 8 | NH-PEG₆-Phe-CO | 338 |

Here, from the Table 2, it is clearly visible that only the presence of Phe-Phe does not make the bioconjugate having high affinity towards PSMA targeted cells. As in the present invention, with the AAPT targeting ligand and Phe-Phe, binding affinity is much better. In case of DUPA conjugates, it is mentioned that the binding affinity is lost when the chain length of aminocaprylic acid is modified or absence or presence of three phenyl rings in the spacer results in considerable loss of binding affinities for eg., (a) DUPA conjugate with spacer NH-Phe-Glu-Ala-Phe-Phe-CO has Kd=102 nM (compound 7 in Table 3) and (b) with spacer length NH-PEG₆-Phe-CO, Kd=338 nm (compound 8 in table 3) (c) no phenyl ring Kd=176 nm (compound 3 in Table 3).

This makes PEG an adverse candidate to choose to make a bioconjugate for PSMA targeted cells. However, in the present invention, in FIG. 22, AA1, AA2, AA3 and AA4, it has been shown that PEG is there, still the binding affinities of 8a-8g (FIG. 24-30) has been shown≤60 nM which is more than 6 times better binding affinity. This drastic change in the binding affinity is due to unique combination of amino acids and bonds in AAPT targeting ligand, spacer, linker or the chelating ligand.

In an embodiment, in FIG. 22, a general SPPS method for preparation of AAPT PCa bioconjugate 8a-8g has been shown.

In 8a, AA₁=trans-4-aminomethylcyclohexanecarboxylic acid, AA₂=2-naphthylalanine, AA₃=2-naphthylalanine, AA₄=NH₂—(CH₂)2-PEG₃-(CH₂)2CO₂H.

In 8b, AA₁=trans-4-aminomethylcyclohexanecarboxylic acid, AA₂=2-naphthylalanine, AA₃=phenylalanine, AA4=NH₂—(CH₂)2-PEG₃-(CH₂)2CO₂H.

In 8c, AA₁=2-naphthylalanine, AA₂=trans-4-aminomethylcyclohexanecarboxylic acid, AA₃=2-naphthylalanine, AA4=NH₂—(CH₂)2-PEG₃-(CH₂)2CO₂H.

In 8d, AA1=trans-4-aminomethylcyclohexanecarboxylic acid, AA₂=2-naphthylalanine, AA₃=NH₂—(CH₂)2-PEG₃-(CH₂)2CO₂H.

In 8e, AA1=2-naphthylalanine, AA₂=2-naphthylalanine, AA₃=2-naphthylalanine, AA₄=NH₂—(CH₂)2-PEG₃-(CH₂)2CO₂H.

In 8f, AA1=2-naphthylalanine, AA₂=2-naphthylalanine, AA₃=trans-4-aminomethylcyclohexanecarboxylic acid, AA₄=NH₂—(CH₂)2-PEG₃-(CH₂)2CO₂H.

In 8g, AA1=2-naphthylalanine, AA₂=2-naphthylalanine, AA₃=NH₂—(CH₂)7-(CH₂)2CO₂H

The conjugate of claim 1, wherein AA2 comprises 2-naphthylalanine or trans-4-aminomethylcyclohexanecarboxylic acid.

The conjugate of claim 1, wherein AA3 comprises 2-naphthylalanine or phenylalanine or NH₂—(CH₂)2-PEG_{2/3}-(CH₂)2CO₂H or NH₂—(CH₂)7-CO₂H.

The conjugate of claim 1, wherein AA4 comprises NH₂—(CH₂)2-PEG_{2/3}-(CH₂)2CO₂H.

In an embodiment, FIG. 23 represents the general structure of AAPT_PCa biocongugate with various spacers, wherein AA1, AA2, AA3 and/or AA4 amino acids differ from each other and therefore, different binding affinity.

In an embodiment, the conjugates having formula:

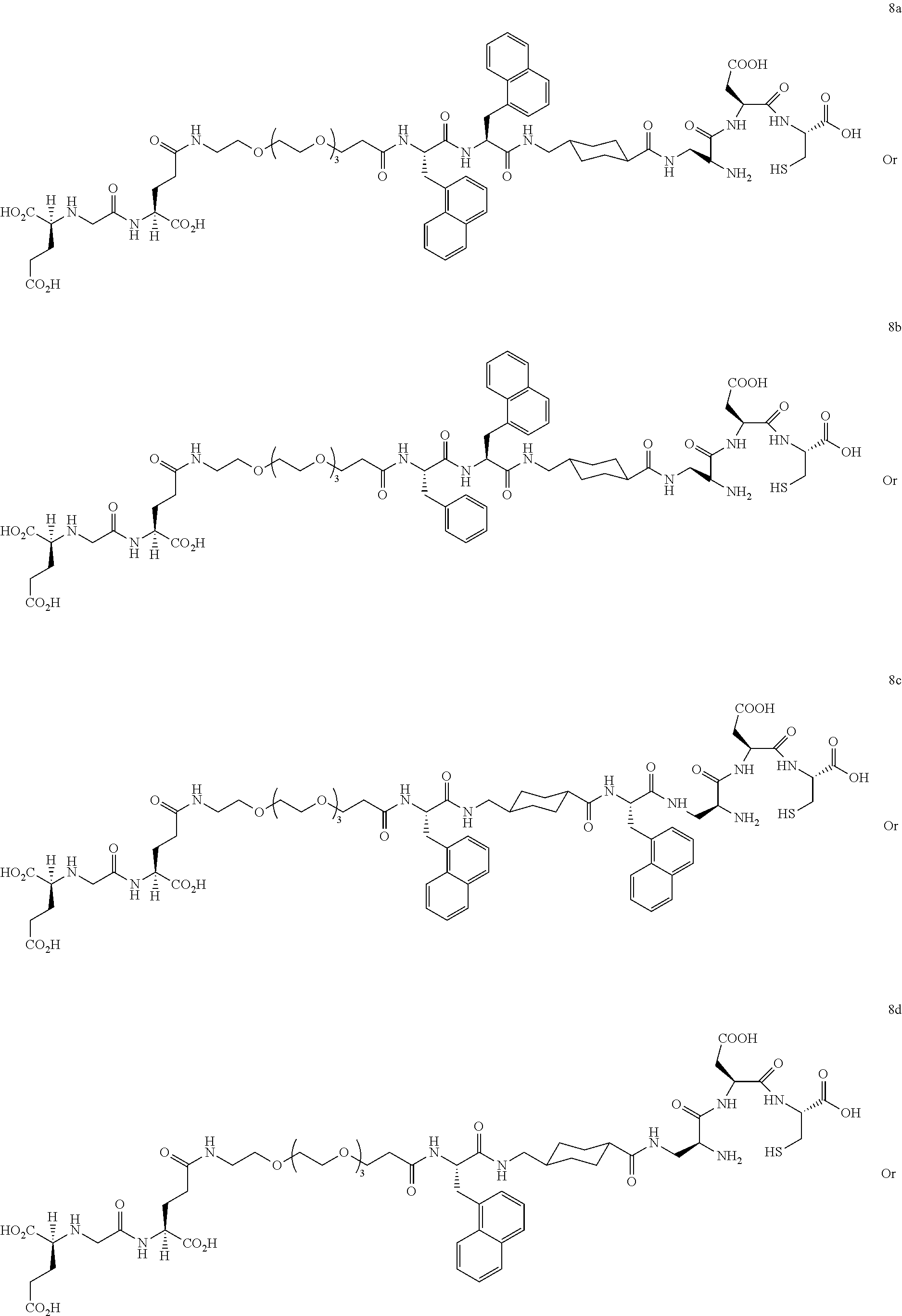

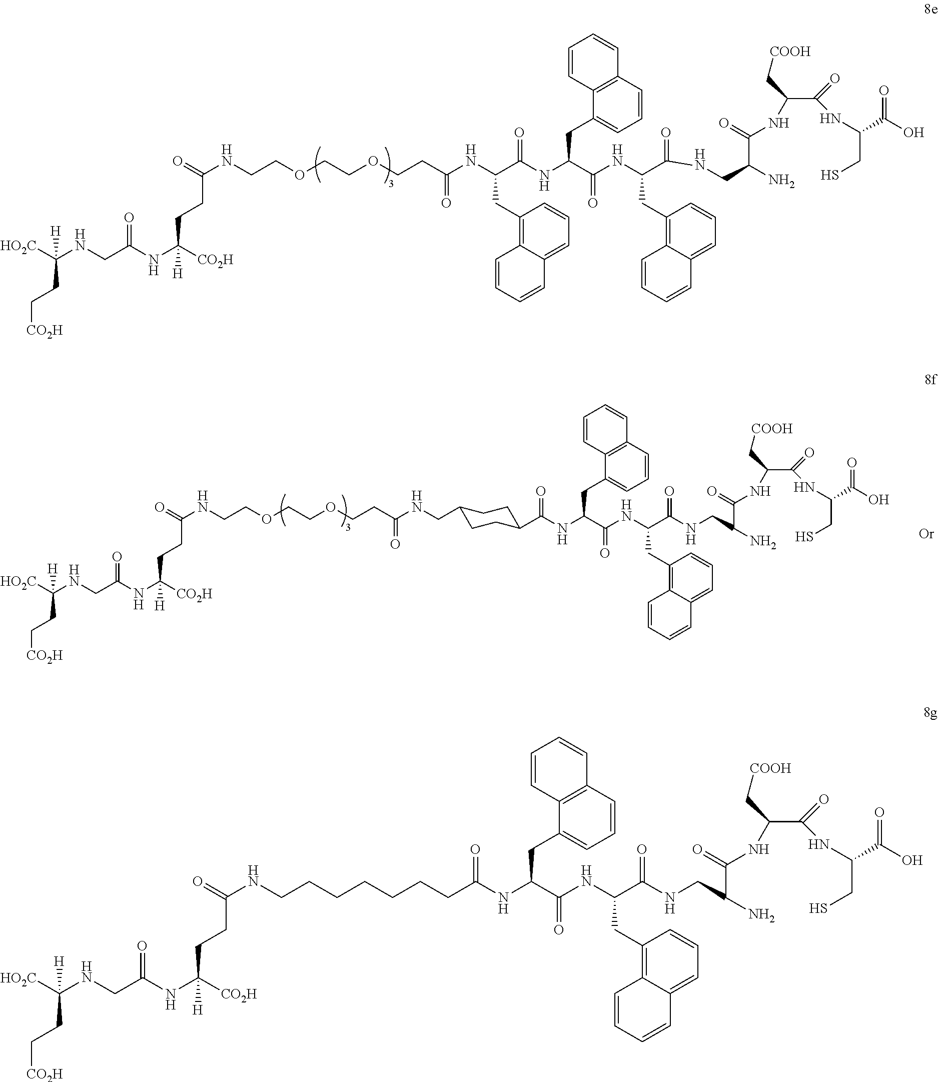

In an embodiment, the conjugate is configured to be used for targeted drug delivery of radioisotopes for diagnosis and treatment of malignancy and neurodegenerative diseases where PSMA protein is overexpressed.

In an embodiment, the amino acid residues of E are optionally substituted.

In an embodiment, FIG. 24-30 represents evaluation of binding affinity of AAPT-PCa Bioconjugate 8a-8g in PSMA+ LNCaP cells.

An embodiment relates to a method of preparation of precursor of AAPT ligand as following (FIG. 21):
(a) stirring a solution of protected amino acids (1 equiv.) and NaHCO₃ (1.2 equiv.) in CH₂Cl₂:H₂O (5v:0.5v or 1v:1v) and cooling to 0° C. to get a reaction mixture;
(b) adding α-Chloroacetyl chloride (1.2 equiv.) dropwise over a period of 15 minutes at 0° C. to the reaction mixture of step (a) and stirring the reaction mixture for 1 h by allowing the temperature to raise to room temperature and further stirring for 1 h or 2 h;
(c) diluting the reaction mixture of step (b) with water and dichloromethane and separating an organic layer and washing it with sodium bicarbonate solution;
(d) drying the organic layer of step (c) over anhydrous sodium sulfate, filtering and concentrating under reduced pressure to get (S)-tert-Butyl 2-(2-chloroacetamido)-3-phenyl ester derivatives 13a-c in 78-96% yields;
(e) reacting (S)-tert-Butyl 2-(2-chloroacetamido)-3-phenyl ester derivative of step (d) with an amino acid derivatives 14a-j in the presence of potassium iodide and K₂CO₃ in refluxing dry THF overnight to get precursors 15a-j of AAPT ligands in 82-94% yields;
(f) characterizing the precursors of AAPT ligand precursors of using various spectroscopic techniques.

In an embodiment. (S)-tert-Butyl 2-(2-chloroacetamido)-3-phenyl ester derivative is any of the 13a-c (FIG. 21).

In an embodiment, bromo derivative of 13a-c can also be used here.

In an embodiment, an amine derivative in the method is 14a-j (FIG. 21).

In an embodiment, in FIG. 21, R₁ group of 15a-j is from 13a-c (or initially from 11a-c) and R₂ group of 15a-j is from 14a-j.

In an embodiment, R₁ group is selected from CH₂CH₂COOBn, CH₂C6H₄—OH-p and CH₂C6H₅.

In an embodiment, R₂ group is selected from CH₂CH₂COOBu^{t}, CH₂C6H₄—OH-p, CH₂C6H₅, CH₃, CH(CH₃)₂, CH₂CH(CH₃)₂, CH(CH₃)CH₂CH₃, CH₂OBu^{t}, CH₂CH₂COOBu^{t}, CH₂CH₂COOBu^{t}.

After the completion of molecular docking study, inhibitors 1-3 were chemically synthesized by deprotection of carboxy protecting groups (benzyloxy and tertiary-butyl) from their precursors 15a-c and selected for further in vitro biological evaluation to inhibit GCPII enzyme activity. Carboxy protected acetamide precursors 15d-j of acetamide based GCPII inhibitors 4-10 were also synthesized and completely characterized for future studies if required. As per molecular docking studies GCPII inhibitors 4-10 (Table 2) had poor docking scores when compared to inhibitors 1-3 and hence not synthesized chemically in the lab though their precursors 15d-j are synthesized, characterized using various spectroscopic techniques and readily available for further experimental studies.

In an embodiment, the method of preparation of precursor of AAPT ligand further comprises a method of solid phase synthesis of AAPT ligand conjugated with a spacer (FIG. 22) is as following:
(a) swelling of H-Cys-2-ClTrt resin (0.80-0.100 g) in a resin beads in CH₂Cl₂ (5-10 mL) for 30 minutes by bubbling nitrogen and after draining CH₂Cl₂, the resin is swelled once again with DMF (5 mL×3) thrice for 15 minutes each and then (i) adding Fmoc-Asp(O^{t}Bu)-OH (90-100 mg), PyBOP (110-120 mg) and DIPEA (0.1-0.2 mL) in DMF (0.5 mL) to the peptide vessel and continuing the coupling reaction for 6 h and then washing the resin with DMF thrice following by isopropanol thrice to perform a reaction; (ii) confirming the completion of the reaction by performing Kaiser test and then adding 20% Piperidine in DMF at room temperature and bubbling the resin beads for 30 min;
(b) adding Fmoc-diaminopropionic (DAP) acid, PyBOP and DIPEA in DMF for 6 h and then adding 20% Piperdine in DMF at room temperature and bubbling the resin beads for 30 min;
(c) adding Fmoc-AA1-OH, PyBOP and DIPEA in DMF for 6 h and then adding 20% Piperidine in DMF at room temperature and bubbling the resin beads for 30 min;
(d) adding Fmoc-AA₂-OH, PyBOP and DIPEA in DMF for 6 h and then adding 20% Piperidine in DMF at room temperature and bubbling the resin beads for 30 min;
(e) adding Fmoc-AA₃-OH, PyBOP and DIPEA in DMF for 6 h and then adding 20% Piperidine in DMF at room temperature and bubbling the resin beads for 30 min;
(f) adding Fmoc-AA₄-OH, PyBOP and DIPEA in DMF for 6 h and then adding 20% Piperidine in DMF at room temperature and bubbling the resin beads for 30 min;
(g) adding AAPT(O^{t}Bu)₃-OH, PyBOP, and DIPEA in DMF to the peptide vessel of step (f) for 6 h;
(h) adding 5 ml of a mixture of TFA to the resin beads and bubbling nitrogen gas through resin beads for 30 min and then 2.5 ml of the mixture of TFA for 15 min twice and collecting a mother liquor in a round bottom flask and then evaporating the mixture of TFA from the mother liquor to form concentrated viscous mixture;
(i) precipitating the AAPT ligand conjugated with the spacer from the concentrated viscous mixture of step (h) in ice cold diethylether.

In an embodiment, a mixture of TFA comprises TFA/H₂O/TIPS/EDT (92.5:2.5:2.5:2.5) or TFA/H₂O/TIPS (95:2.5:2.5).

An embodiment relates to a conjugate comprising:
a) a ligand;
b) a spacer; and
c) a chelating ligand;
wherein the ligand is a compound of Formula I

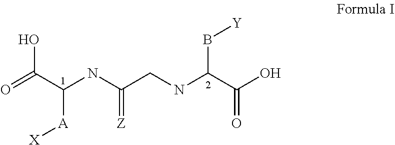

and stereoisomers thereof, wherein A and B are independently selected from a group consisting of hydrogen, optionally substituted C₁-C₇ alkyl, and optionally substituted aryl groups; X and Y are selected from the groups comprising of —H, —OH, and —COOH groups, and Z is one of O or S groups; wherein the conjugate is a AAPT-PCa DOTA bioconjugate.
wherein A and B are independently selected from a group consisting of hydrogen, C₁-C₃ alkyl, and aryl groups; X and Y are selected from the groups comprising of —H, —OH, and —COOH groups, Z is a O group.

The conjugate as claimed in claim 1, wherein stereochemical configuration of the stereocenter 1 and 2 of the compound of Formula I is of S configuration.

In an embodiment, the conjugate comprises targeting ligand or AAPT ligand, linker, spacer, and the chelating ligand.

In an embodiment, the linker is a PEG linker.

In an embodiment, the spacer is a peptide comprising at least 2-20 amino acids.

In an embodiment, the chelating ligand is attached to a drug which is at least one of imaging agents, anticancer drug or a radionuclide.

In an embodiment, the conjugate comprises AAPT-PCa DOTA bioconjugate having formula:

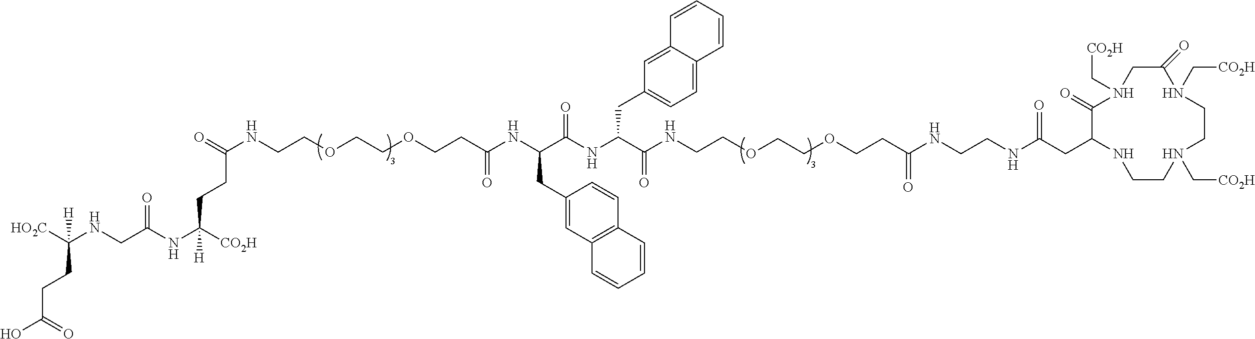

In an embodiment, a method of solid phase synthesis of AAPT ligand conjugated arene chelating linker (FIG. 31) is as following:
(a) swelling of H-Cys-2-ClTrt resin (0.100 g, 0.09 mmol) in resin beads in CH₂Cl₂ (5 mL) for 30 minutes by bubbling nitrogen and after draining CH₂Cl₂, the resin is swelled once again with DMF (5 mL×3) thrice for 15 minutes each;
(b) adding Fmoc-Asp(OtBu)-OH (93 mg, 0.225 mmol), PyBOP (116 mg, 0.225 mmol) and DIPEA (0.16 mL, 0.90 mmol) in DMF (0.5 mL) to the peptide vessel and continuing the coupling reaction for 6 h;
(c) washing the resin with DMF thrice following by isopropanol thrice to perform a reaction;
(d) confirming the completion of the reaction by performing Kaiser test and then adding 20% Piperidine in DMF at room temperature and bubbling the resin beads for 30 min;
(e) attaching Fmoc-diaminopropionic (DAP) acid, Fmoc-NH—(PEG)₃-COOH, 4-(Fmoc-aminomethyl)benzoic acid, Fmoc-2-Nal-OH, Fmoc-2-Nal-OH, Fmoc-NH—(PEG)₃-COOH by following step (b) to step (d) replacing Fmoc-Asp(OtBu)-OH.
(f) adding AAPT(OtBu)₃-OH, PyBOP, and DIPEA in DMF to the peptide vessel of step (e) for 6 h;
(g) adding 5 ml of a mixture of TFA to the resin beads and bubbling nitrogen gas through resin beads for 30 min and then 2.5 ml of the mixture of TFA for 15 min twice and collecting a mother liquor in a round bottom flask;
(h) evaporating the mixture of TFA from the mother liquor to form concentrated viscous mixture;
(i) precipitating the AAPT ligand conjugated with the spacer from the concentrated viscous mixture of step (h) in ice cold diethylether.

In an embodiment, a method of solid phase synthesis of AAPT-PCa DOTA bioconjugate (FIG. 32), for delivering of radioisotopes comprising:
(a) swelling of Universal NovaTag resin in resin beads in a peptide vessel in 20% Piperidine in DMF at room temperature and bubbling the resin beads for 30 min and then adding Fmoc-NH—(PEG)₃-CO₂H, PyBOP and DIPEA in DMF to the peptide vessel and continuing the coupling reaction for 6 h and washing the resin with DMF thrice following by isopropanol thrice to perform a reaction;
(b) confirming the completion of the reaction by performing Kaiser test and then adding 20% Piperidine in DMF at room temperature and bubbling the resin beads for 30 min and adding Fmoc-2-Nal-OH twice, PyBOP and DIPEA in DMF for 6 h;
(c) confirming the completion of the reaction by performing Kaiser test and then adding 20% Piperidine in DMF at room temperature and bubbling the resin beads for 30 min and adding Fmoc-NH—(PEG)₃-COOH PyBOP and DIPEA in DMF for 6 h;
(d) adding 20% Piperidine in DMF at room temperature and bubbling the resin beads for 30 min and adding AAPT(O^{t}Bu)₃-OH, PyBOP, and DIPEA in DMF to the peptide vessel of step (c) for 6 h;
(e) adding 1M HOBt in DCM/TFE (1:1) for 10 min at room temperature and then adding DOTA-tris(tert-butyl ester), PyBOP and DIPEA in DMF for 6 h;
(f) adding 5 ml of a mixture of TFA to the resin beads and bubbling nitrogen gas through resin beads for 30 min and then 5 ml of the mixture of TFA for 5 min twice and collecting a mother liquor in a round bottom flask;
(g) evaporating the mixture of TFA from the mother liquor to form concentrated viscous mixture;
(h) precipitating the AAPT-PCa DOTA bioconjugate from the concentrated viscous mixture of step (g) in ice cold diethylether.

In an embodiment, the method for delivering of radioisotopes comprising ⁶⁸Ga and ¹⁷⁷Lu as radioisotopes.

In an embodiment, different chelating linker for ⁶⁸Ga and ¹⁷⁷Lu are used.

### EXAMPLES

### Example 1: General Procedure for the Synthesis of 2-Bromoacetamide Intermediates 13a-c

Bromoacetic acid 12 (0.208 mg, 1.5 mmol), dicylohexylcarbodiimide (0.619, 3.0 mmol) were dissolved in freshly distilled dichloromethane (8 mL), and the resulting mixture was stirred at 0° C. for 30 min. A solution of 11a-c (1.0 mmol) in dichloromethane (5 mL) was added to the reaction mixture. The reaction mixture was stirred for 12 h at room temperature. The progress of the reaction was monitored by thin layer chromatography (TLC). After completion of the reaction, dichloromethane was evaporated under reduced pressure and ethyl acetate was added to the residue of the crude reaction mixture. Dicyclohexyl urea (DCU) was filtered off from the reaction mixture through glass funnel by using Whatman filter paper. The ethyl acetate layer was concentrated under reduced pressure and the crude products 13a-c were purified through column chromatography using distilled 15-25% ethyl acetate in hexane.

### Example 2: (S)-5-Benzyl 1-tert-butyl 2-(2-bromoacetamido) pentanedioate (13a)

Yellowish gummy liquid (yield=60%), Rf=0.56 (EtOAc: hexane=1:4); IR (CH2Cl2): 3322 (N—H), 3032, 2975 (═C—H), 2928 (C—H), 1729 (C═O), 1652 (N—H), 1537 (C═C), 1454 (C—H), 1166 (C—O), 750, 699 (═C—H) cm-1. ¹H NMR (400 MHz, CDCl₃): δ 7.37-7.31 (m, 5H), 7.05 (d, J=7.28 Hz, 1H), 5.13, 5.10 (ABquartet, J=13.28 Hz, 2H), 4.49 (ddd, J=7.28, 5.24, 5.14 Hz, 1H), 3.83 (s, 2H), 2.49-2.38 (m, 2H), 2.27-2.20 (m, 1H), 2.07-1.99 (m, 1H), 1.46 (s, 9H). ¹³C NMR (100 MHz, CDCl₃): δ 172.5, 170.3, 165.8, 135.6, 128.6, 128.3, 128.2, 83.0, 66.6, 52.8, 30.1, 28.6, 27.9, 27.3. HRMS (ESI) m/z [M+Na]+ calcd. for C₁₈H₂₄BrNO₅, 436.0730, found, 436.0766.

### Example 3: (S)-tert-Butyl-2-(2-bromoacetamido)-3-(4-hydroxyphenyl) propanoate (13b)

Colourless gummy liquid (yield=70%), Rf=0.4 (EtOAc hexane=1:4); IR (CH2Cl2): 3341 (0-H), 3275 (N—H), 2979 (=C—H), 2933 (C—H), 1733 (C═0), 1657 (N—H), 1518 (C═C), 1456 (C—H), 1155 (C-0), 750, 698 (=C—H) cm-1. ¹H NMR (400 MHz, CDCl₃): δ 7.03 (d, J=8.44 Hz, 2H), 6.93 (d, J=7.32 Hz, 1H), 6.73 (d, J=8.44 Hz, 2H), 5.96 (brs, 1H), 4.71-4.63 (m, 1H), 3.85, 3.81 (ABquartet, J=13.80 Hz, 2H), 3.09-2.96 (m, 2H), 1.43 (s, 9H). ¹³C NMR (100 MHz, CDCl₃): δ 170.1, 165.3, 155.2, 130.6, 127.2, 115.4, 82.9, 54.3, 37.1, 28.7, 27.9. HRMS (ESI) m/z [M+Na]+ calcd. for C₁₅H₂₀BrNO₄, 380.0468, found, 380.0479.

### Example 4: (S)-tert-Butyl 2-(2-bromoacetamido)-3-phenylpropanoate (13c)

Colourless gummy liquid (yield=65%), Rf=0.52 (EtOAc hexane=1:4); IR (CH₂Cl₂): 3298 (N—H), 2979 (═C—H), 2933 (C—H), 1734 (C═0), 1657 (N—H), 1528 (C═C), 1456 (C—H), 1155 (C-0), 740 (═C—H) cm-1. ¹H NMR (400 MHz, CDCl₃): δ 7.31-7.26 (m, 3H), 7.17-7.15 (m, 2H), 6.89 (d, J=6.52 Hz, 1H), 4.72 (ddd, J=6.52, 6.0, 4.52 Hz, 1H), 3.87, 3.83 (ABquartet, J=13.80 Hz, 2H), 3.12 (d, J=6.04 Hz, 2H), 1.42 (s, 9H). ¹³C NMR (100 MHz, CDCl₃): δ 169.9, 164.9, 135.7, 129.5, 128.4, 127.1, 82.8, 54.1, 37.9, 28.8, 27.9. HRMS (ESI) m/z [M+Na]+ calcd. for C₁₈H₂₀BrNO₃, 364.0519, found, 364.0522.

### Example 5: General Procedure for the Synthesis of Protected Amino Acetamide Derivatives 15a-j

Compounds 13a-c (1.0 mmol) and 14a-j (1.0 mmol) were dissolved in dry THF (5 mL) and DIPEA (0.52 mL, 3.0 mmol) was added in the reaction mixture. The reaction mixture was refluxed at 80° C. for 18-20 h. The reaction progress was monitored through TLC. After the completion of the reaction, THF was evaporated under reduced pressure and 30 mL of ethyl acetate was added to the crude reaction mixture. The organic layer was washed with distilled water (2×15 mL) and the resultant organic layer was dried over anhydrous sodium sulphate. The organic layer was concentrated under reduced pressure and the crude products 15a-j were purified through column chromatography using distilled 33% ethyl acetate and hexane mixture as eluent.

### Example 6: (S)-5-Benzyl-1-tert-butyl-2-(2-(((S)-1,5-di-tert-butoxy-1,5-dioxopentan-2-yl)amino)acetamido)pentanedioate (15a)

Yellowish gummy liquid (yield=75%), Rf=0.3 (EtOAc hexane=1:2), IR (CH₂Cl₂): 3349 (N—H), 2978 (═C—H), 2928 (C—H), 1729 (C═0), 1682 (N—H), 1517 (C═C), 1456 (C—H), 1155 (C-0), 750, 699 (═C—H) cm-1. ¹H NMR (400 MHz, CDCl₃): δ 7.70 (d, J=8.4 Hz, 1H), 7.40-7.26 (m, 5H), 5.10 (s, 2H), 4.52 (ddd, J=7.99, 5.66, 4.76, 1H), 3.39 (d, J=17.2 Hz, 1H), 3.13-3.05 (m, 1H), 3.00 (d, J=17.2 Hz, 1H), 2.48-2.32 (m, 4H), 2.28-2.18 (m, 1H), 2.04-1.91 (m, 3H), 1.87-1.75 (m, 1H), 1.45 (s, 9H), 1.44 (s, 9H), 1.42 (s, 9H). ¹³C NMR (100 MHz, CDCl₃): δ 173.7, 172.6, 171.3, 170.7, 135.9, 128.6, 128.3, 82.2, 81.9, 80.5, 66.4, 61.9, 51.7, 51.0, 32.4, 30.5, 28.7, 28.1, 28.0, 27.6. HRMS (ESI) m/z [M+Na]+ calcd. for C₃₁H₄₈N₂O₉, 615.3252, found, 615.3308.

### Example 7: (S)-5-Benzyl-1-tert-butyl-2-(2-(((S)-1-(tert-butoxy)-3-(4-hydroxyphenyl)-1-oxopropan-2-yl)amino)acetamido)pentanedioate (15b)

Gummy liquid (yield=75%), Rf=0.25 (EtOAc: hexane=1:2); IR (CH2Cl2): 3321 (0-H), 3279 (N—H), 3067, 2979 (═C—H), 2929, 2851 (C—H), 1731 (C═0), 1650 (N—H), 1537, 1517 (C═C), 1448 (C—H), 1154 (C-0), 750, 699 (═C—H) cm-1. ¹H NMR (400 MHz, CDCl₃): δ 7.40-7.32 (m, 5H), 7.07 (d, J=7.52 Hz, 3H), 6.70 (d, J=7.52 Hz, 2H), 5.15, 5.10 (ABquartet, J=12.28 Hz, 2H), 4.31-4.26 (m, 1H), 3.36 (d, J=17.56 Hz, 1H), 3.33-3.25 (m, 1H), 3.00 (d, J=17.56 Hz, 1H), 2.93 (dd, J=13.8, 4.52 Hz, 1H), 2.68 (dd, J=12.80, 9.28 Hz, 1H), 2.40-2.21 (m, 2H), 2.10-1.95 (m, 1H), 1.87-1.58 (m, 2H), 1.44 (s, 9H), 1.41 (s, 9H). ¹³C NMR (100 MHz, CDCl₃): δ 173.4, 172.4, 171.3, 170.6, 137.2, 135.8, 129.6, 128.6, 128.4, 128.3, 126.7, 82.1, 81.8, 66.4, 63.2, 51.5, 50.6, 39.9, 30.5, 28.0, 27.9, 27.3. HRMS (ESI) m/z [M+H]⁺ calcd. for C₃₁H₄₂N₂O₈, 571.3014, found, 571.3007.

### Example 8: (S)-5-Benzyl-1-tert-butyl-2-(2-(((S)-1-(tert-butoxy)-1-oxo-3-phenylpropan-2-yl)amino)acetamido)pentanedioate (15c)

White solid (yield=70%), Rf=0.35 (EtOAc: hexane=1:2); IR (CH2Cl2): 3326 (N—H), 2979 (═C—H), 2929 (C—H), 1723 (C═0), 1668 (N—H), 1520 (C═C), 1455 (C—H), 1154 (C-0), 735, 698 (═C—H) cm-1. 1H NMR (400 MHz, CDCl₃): δ 7.40-7.34 (m, 5H), 7.28-7.20 (m, 5H), 5.13, 5.09 (ABquartet, J=14.04 Hz, 2H), 4.45-4.35 (m, 1H), 3.41-3.34 (m, 2H), 3.07-2.84 (m, 3H), 2.43-2.24 (m, 2H), 2.18-2.06 (m, 1H), 2.04-1.85 (m, 1H), 1.82-1.70 (m, 1H), 1.46 (s, 9H), 1.37 (s, 9H), 1.25-1.21 (m, 1H). 13C NMR (100 MHz, CDCl₃): δ 173.7, 172.9, 171.9, 170.4, 155.0, 135.7, 130.6, 128.9, 128.6, 128.4, 128.3, 115.7, 82.1, 81.8, 66.6, 63.5, 51.7, 50.5, 38.9, 30.6, 28.0, 27.9, 26.9. HRMS (ESI) m/z [M+Na]⁺ calcd. for C₃₁H₄₂N₂O₇, 577.2884, found, 577.2888.

### Example 9: (S)-5-Benzyl-1-tert-butyl-2-(2-(((S)-1-(tert-butoxy)-1-oxopropan-2-yl)amino) acetamido)pentanedioate (15d)

White solid (yield=72%), Rf=0.32 (EtOAc: hexane=1:2); IR (CH2Cl2): 3338 (N—H), 2974 (═C—H), 2927 (C—H), 1730 (C═0), 1666 (N—H), 1523 (C═C), 1455 (C—H), 1152, 1064 (C-0), 734, 697 (═C—H) cm-1. 1H NMR (400 MHz, CDCl₃): δ 7.77 (d, J=8.52 Hz, 1H), 7.36-7.30 (m, 5H), 5.12, 5.08 (ABquartet, J=13.04 Hz, 2H), 4.53 (ddd, J=8.52, 5.78, 5.0 Hz, 1H), 3.37 (d, J=17.32 Hz, 1H), 3.16 (q, J=7.04 Hz, 1H), 3.04 (d, J=17.32 Hz, 1H), 2.52-2.32 (m, 2H), 2.27-2.15 (m, 1H), 2.04-1.92 (m, 1H), 1.45 (s, 9H), 1.44 (s, 9H), 1.28 (d, J=7.04 Hz, 3H), 1.24 (brs, 1H). 13C NMR (100 MHz, CDCl₃): δ 174.5, 172.6, 171.4, 170.8, 135.8, 128.6, 128.3, 128.2, 82.3, 81.5, 66.5, 57.5, 51.6, 50.8, 30.4, 28.0, 27.9, 27.8, 19.3. HRMS (ESI) m/z [M+Na]⁺ calcd. for C₂₅H₃₈N₂O₇, 501.2571, found, 501.2572.

### Example 10: (S)-5-Benzyl-1-tert-butyl-2-(2-(((S)-1-(tert-butoxy)-3-methyl-1-oxobutan-2-yl)amino)acetamido)pentanedioate (15e)

Yellowish gummy liquid (yield=75%), Rf=0.36 (EtOAc hexane=1:2); IR (CH2Cl2): 3363 (N—H), 2975 (═C—H), 2933 (C—H), 1733 (C═0), 1681 (N—H), 1513 (C═C), 1457 (C—H), 1157 (C-0), 746, 699 (═C—H) cm-1.1H NMR (400 MHz, CDCl₃): δ 7.73 (d, J=8.28 Hz, 1H), 7.38-7.32 (m, 5H), 5.12, 5.08 (ABquartet, J=12.56 Hz, 2H), 4.51 (ddd, J=8.28, 5.66, 5.24, 1H), 3.40 (d, J=17.32 Hz, 1H), 2.98-2.93 (m, 2H), 2.51-2.33 (m, 2H), 2.25-2.15 (m, 1H), 2.05-1.93 (m, 1H), 1.44 (s, 18H), 1.30-1.13 (m, 2H), 0.97-0.86 (m, 6H). ¹³C NMR (100 MHz, CDCl₃): δ 173.9, 172.6, 171.6, 170.7, 135.9, 128.6, *128.4, 82.3, 81.6, 68.0, 66.5, 51.8, 51.3, 31.6, 30.5, 28.2, 28.1, 27.9, 19.5, 18.4. HRMS (ESI) m/z [M+Na]⁺ calcd. for C₂₇H₄₂N₂O₇, 529.2884, found, 529.2882. (* indicates higher intensity carbon)

### Example 11: (S)-5-Benzyl-1-tert-butyl-2-(2-(((S)-1-(tert-butoxy)-4-methyl-1-oxopentan-2-yl)amino)acetamido)pentanedioate (15f)

Yellowish liquid (yield=82%), Rf=0.4 (EtOAc: hexane=1:2); IR (CH2Cl2): 3354 (N—H), 2959 (═C—H), 2934 (C—H), 1734 (C═0), 1681 (N—H), 1511 (C═C), 1456 (C—H), 1155, 1081 (C-0), 749, 699 (═C—H) cm-1.1H NMR (400 MHz, CDCl₃): δ 7.74 (d, J=8.28 Hz, 1H), 7.40-7.30 (m, 5H), 5.11, 5.08 (ABquartet, J=12.80 Hz, 2H), 4.51 (ddd, J=8.28, 5.40, 5.28, 1H), 3.37 (d, J=17.32 Hz, 1H), 3.09 (t, J=7.0 Hz, 1H), 2.99 (d, J=17.32 Hz, 1H), 2.51-2.32 (m, 2H), 2.26-2.15 (m, 1H), 2.04-1.92 (m, 1H), 1.83-1.71 (m, 3H), 1.44 (s, 9H), 1.43 (s, 9H) 1.29-1.22 (m, 1H), 0.92 (d, J=6.52 Hz, 3H), 0.90 (d, J=6.52 Hz, 3H). ¹³C NMR (100 MHz, CDCl₃): δ 174.7, 172.5, 171.4, 170.6, 135.8, 128.6, 128.3, 128.2, 82.2, 81.5, 66.4, 60.9, 51.7, 50.8, 42.9, 30.4, 28.1, 27.9, 27.8, 24.9, 22.6, 22.5. HRMS (ESI) m/z [M+Na]⁺ calcd. for C₂₈H₄₄N₂O₇, 521.3221, found, 521.3222.

### Example 12: (2S)-5-Benzyl-1-tert-butyl-2-(2-(((2S)-1-(tert-butoxy)-3-methyl-1-oxopentan-2-yl)amino)acetamido)pentanedioate (15g)

Yellowish solid (yield=86%), Rf=0.34 (EtOAc: hexane=1:2); IR (CH2Cl2): 3347 (N—H), 2977 (═C—H), 2933 (C—H), 1727 (C═0), 1668 (N—H), 1515 (C═C), 1458 (C—H), 1153 (C-0), 734, 698 (═C—H) cm-1.1H NMR (400 MHz, CDCl₃): δ 7.72 (d, J=8.52 Hz, 1H), 7.42-7.27 (m, 5H), 5.11, 5.08 (ABquartet, J=12.52 Hz, 2H), 4.51 (ddd, J=8.52, 5.38, 5.24, 1H), 3.40 (d, J=17.32 Hz, 1H), 2.95 (d, 1H, J=17.32 Hz), 2.86 (d, J=5.76 Hz, 1H), 2.51-2.32 (m, 2H), 2.26-2.14 (m, 1H), 2.04-1.87 (m, 4H), 1.44 (s, 18H), 1.28-1.23 (m, 1H), 0.99 (d, J=6.80 Hz, 3H), 0.95 (d, J=7.04 Hz, 3H). ¹³C NMR (100 MHz, CDCl₃): δ 173.6, 172.6, 171.5, 170.6, 135.8, 128.6, 128.3, 128.2, 82.2, 81.5, 66.8, 66.5, 51.7, 51.3, 38.5, 30.4, 28.2, 27.9, 27.8, 25.5, 15.7, 11.7. HRMS (ESI) m/z [M+Na]⁺ calcd. for C₂₈H₄₄N₂O₇, 521.3221, found, 521.3223.

### Example 13: (S)-5-Benzyl-1-tert-butyl-2-(2-(((S)-1,4-di-tert-butoxy-1,4-dioxobutan-2-yl)amino)acetamido)pentanedioate (15 h)

Yellowish liquid (yield=85%), Rf=0.32 (EtOAc: hexane=1:2); IR (CH2Cl2): 3327 (N—H), 2975 (═C—H), 2926 (C—H), 1723 (C═O), 1672 (N—H), 1519 (C═C), 1456 (C—H), 1149 (C—O), 750, 698 (═C—H) cm-1.1H NMR (400 MHz, CDCl₃): δ 8.00 (d, J=8.52 Hz, 1H), 7.40-7.27 (m, 5H), 5.11, 5.08 (ABquartet, J=12.80 Hz, 2H), 4.50 (ddd, J=8.52, 6.14, 4.76 Hz, 1H), 3.48-3.40 (m, 2H), 3.18-3.14 (d, J=17.32 Hz, 1H), 2.66 (dd, J=16.7, 4.24, Hz, 1H), 2.57-2.37 (m, 3H), 2.30-2.17 (m, 1H), 2.10-1.96 (m, 1H), 1.44 (s, 9H), 1.43 (s, 9H), 1.41 (s, 9H), 1.27-1.24 (m, 1H). ¹³C NMR (100 MHz, CDCl₃): δ 172.6, 172.5, 171.6, 170.7, 170.4, 135.9, 128.5, 128.3, 128.2, 82.0, 81.9, 81.5, 66.4, 58.1, 51.8, 50.9, 38.8, 30.6, 28.1, *27.9, 27.2. HRMS (ESI) m/z [M+Na]⁺ calcd. for C₃₀H₄₆N₂O₉, 578.3203, found, 579.3275. (* indicates higher intensity carbon).

### Example 14: (S)-Di-tert-butyl-2-(2-(((S)-1-(tert-butoxy)-3-(4-hydroxyphenyl)-1-oxopropan-2-yl)amino)-2-oxoethyl)amino) pentanedioate (15i)

White solid (yield=85%), Rf=0.25 (EtOAc: hexane=1:2); IR (CH2Cl2): 3349 (0-H), 3275 (N—H), 2979 (═C—H), 2933 (C—H), 1730 (C═O), 1660 (N—H), 1517 (C═C), 1456 (C—H), 1155 (C-0), 753 (═C—H) cm-1. ¹H NMR (400 MHz, CDCl₃): δ 7.57 (d, J=8.44 Hz, 1H), 7.01 (d, J=8.08 Hz, 2H), 6.72 (d, J=8.08 Hz, 2H), 4.75-4.65 (m, 1H), 3.35 (d, J 16.88 Hz, 1H), 3.10-2.90 (m, 4H), 2.37-2.20 (m, 2H), 1.90-1.67 (m, 2H), 1.43 (s, 18H), 1.42 (s, 9H), 1.27-1.22 (m, 1H). ¹³C NMR (100 MHz, CDCl₃): δ 173.6, 172.8, 171.2, 170.6, 155.3, 130.4, 127.7, 115.7, 82.1, 81.9, 80.8, 61.6, 53.3, 50.8, 37.2, 32.3, 28.7, 28.7, 28.09, 28.06, 28.0. HRMS (ESI) m/z [M+Na]⁺ calcd. for C₂₈H₄₄N₂O₈, 559.2990, found, 559.2983.

### Example 15: (S)-Di-tert-butyl-2-((2-(((S)-1-(tert-butoxy)-1-oxo-3-phenylpropan-2-yl)amino)-2-oxoethyl)amino)pentanedioate (15j)

White solid (yield=80%), Rf=0.30 (EtOAc: hexane=1:2); IR (CH2Cl2): 3350 (N—H), 2979 (═C—H), 2933 (C—H), 1732 (C═0), 1682 (N—H), 1518 (C═C), 1456 (C—H), 1155, 1080 (C-0), 741 (═C—H) cm-1. 1H NMR (400 MHz, CDCl₃): δ 7.54 (d, J=8.44 Hz, 1H), 7.30-7.15 (m, 5H), 4.80-4.65 (m, 1H), 3.36 (d, J 17.24 Hz, 1H), 3.15-3.07 (m, 2H), 3.07-3.01 (m, 1H), 2.98 (d, J=17.24 Hz, 1H), 2.35-2.17 (m, 2H), 1.92-1.83 (m, 1H), 1.78-1.67 (m, 2H), 1.44 (s, 9H), 1.43 (s, 9H), 1.39 (s, 9H). ¹³C NMR (100 MHz, CDCl₃): δ 173.7, 172.5, 170.8, 170.5, 136.5, 129.4, 128.4, 126.9, 82.0, 81.8, 80.4, 61.6, 53.2, 50.9, 37.9, 32.3, 28.6, 28.1, 28.0, 27.9. HRMS (ESI) m/z [M+H]⁺ calcd. for C₂₈H₄₄N₂O₇, 521.3221, found, 521.3226.

### Example 16: Deprotection of Carboxylic Benzylester Precursors 15a-c to Afford Tert-Butylcarboxylic Acids 16a-c

Compound 15a-c (1.0 mmol) was dissolved in MeOH (5 mL) in a 50 mL two-neck round bottom flask, 10% Pd/C (0.106 g, 0.1 mmol) was added in the solution. The reaction mixture was hydrogenated at 1 atm for 24 h at room temperature. After the completion of the reaction, Pd/C was filtered through a celite pad (sintered glass filter was half-filled with celite powder) and washed with ethyl acetate (3×20 mL). The ethyl acetate layer was concentrated under reduced pressure and the crude products 16a-c were purified through column chromatography by using distilled ethyl acetate to obtain pure 16a-c.

### Example 17: (S)-5-(Tert-butoxy)-4-(2-(((S)-1,5-di-tert-butoxy-1,5-dioxo-pentan-2-yl)amino)acetamido)-5-oxopentanoic acid (16a)

Colourless gummy liquid (yield=60%). Rf=0.58 (EtOAc Hexane=1:1); IR (CH2Cl2): 3350 (N—H), 2979 (═C—H), 2933 (C—H), 1732 (C═0), 1682 (N—H), 1518 (C═C), 1456 (C—H), 1155, 1080 (C-0), 741 (═C—H) cm-1. ¹H NMR (400 MHz, CDCl₃): δ 7.81 (d, J=8.44 Hz, 1H), 4.50 (ddd, J=7.79, 6.06, 4.76 Hz, 1H), 3.43 (d, J=16.84 Hz, 1H), 3.15-3.07 (m, 1H), 3.04 (d, J=16.84 Hz, 1H), 2.45-2.35 (m, 4H), 2.27-2.15 (m, 1H), 2.05-2.15 (m, 1H), 2.05-1.92 (m, 2H), 1.90-1.80 (m, 1H), 1.46 (s, 9H), 1.45 (s, 9H), 1.43 (s, 9H), 1.13-1.07 (brs, 1H). ¹³C NMR (100 MHz, CDCl₃): δ 175.9, 173.7, 172.9, 171.8, 170.6, 82.3, 82.0, 80.8, 61.7, 51.9, 50.8, 32.3, 30.5, 28.6, 28.1, 27.9, 27.6. HRMS (ESI) m/z calcd for C24H42N2O9 [M+H]⁺: 503.3004, found. 503.3011.

### Example 18: Procedure for the Deprotection of Tert-Butylcarboxylic Acids in 16a-c to Afford Inhibitors 1-3

Precursors 16a-c (1.0 mmol) was dissolved in CH2Cl2 (2 mL) in a 50 mL round bottomed flask. A mixture of trifluoroacetic acid (2.5 mL) and CH2Cl2 (2.5 mL) in the ratio 1:1 was added to the reaction mixture at room temperature and stirred for 2 h. After the completion of reaction, the mixture of trifluoroacetic acid and CH₂Cl₂ were removed under reduced pressure. The products 1-3 were precipitated by the addition of ice-cold ether (5 mL). The crude products 1-3 were washed (3×5 mL) with ether to remove excess trifluoroacetic acid and other non-polar impurities. The products 1-3 were purified through Buchi Reveleris preparative high-performance liquid chromatography using RP—PFP column (XSelect CSH Prep Fluorophenyl 5 μm OBD, 19 mm×150 mm). The purity of the products 1-3 was confirmed by analytical high-performance liquid chromatography and LC-MS. The purified inhibitors 1-3 were used for NAALADase or PSMA enzyme inhibition assay to determine the IC₅₀.

### Analytical HPLC Method

The purity of ligands 1-3 were analyzed using a Dionex HPLC-Ultimate 3000 system. Typically a solution of each ligand (20 μL, 1.0 mg/1.0 mL) in a mixture of CH₃CN:H₂O (1:1) was injected via autosampler and eluted using Dionex Acclaim® 120 C18, 5 m, 4.6 mm×250 mm analytical column at a flow rate of 1 mL/min (mobile phase, A=0.1% trifluoro acetic acid/H₂O and B=acetonitrile). An isocratic flow of 40% B (v/v) was used during the run for 0 to 4 min and gradually gradient of B was increased to 100% B (v/v) over a period of 40-min. The chromatogram of each ligand was recorded on the Ultimate 3000 RS variable wavelength detector at 225-280 nm.

### Example 19: Preparative HPLC Method

The purification of ligands 1-3 was performed using Buchi Reveleris Preparative HPLC System. Crude ligand (20 mg) was dissolved in 1:1 ratio of CH₃CN:H₂O (1 mL) and injected into the sample injector for elution using RP—PFP (Reverse Phase pentafluorophenyl) preparative column (XSelect CSH Prep Fluorophenyl 5 μm OBD, 19 mm×150 mm). A flow rate of 10 mL/min (mobile phase, A=0.1% trifluoro acetic acid/H2O and B=acetonitrile) is maintained throughout the run and the mobile phase gradient was increased from 1% B (v/v) to 50% B (v/v) over a period of 40 min. The mobile phase gradient was further increased to 80% B (v/v) in the next 15 min and the chromatogram was recorded at λ=200-254 nm as well as by ELSD detector. Pure fractions of 1-3 were collected using automatic fraction collector, acetonitrile was evaporated under reduced pressure, lyophilized to afford pure ligands 1-3. The pure ligands were further used for GCPII enzyme inhibition assay.

### Example 20: Procedure for PSMA or GCPII Enzyme Inhibition Assay

Fluorescent-based enzyme inhibition assay was performed to determine the IC₅₀ value of the newly synthesized GCPII inhibitors 1-3 (AAPT ligands). Amplex Glutamate kit was purchased from Invitrogen, and a working solution of Amplex Red reagent (5 mL, 100 μM) was prepared. Meanwhile, membrane portion of PSMA enzyme was extracted from PSMA+ LNCaP cell line by following a reported protocol. Briefly LNCaP cells (1 million) were harvested in HEPES buffer (1 mL) and lyzed twice using probe sonicator for 30 s. The lysate was ultracentrifuged at 100,000×g for 30 min, the supernatant was discarded, and the cell pellet was homogenized by addition of HEPES buffer (1 mL) and used for PSMA enzyme inhibition assay. The isolated enzyme (100 μL, 8.3027 ng) was incubated with different concentrations (1, 5, 10, 25, 50, 75, 90, 100, 200, 300, 500 and 1000 nM) of the inhibitor 1-3 (100 μL) in the presence of N-acetylaspartylglutamate (NAAG) (50 μL, 30 nM) for 60 min. The amount of glutamic acid released by the hydrolysis of NAAG was measured by incubating a working solution of Amplex Red reagent (50 μL, 100 μM) for 30 min at 37° C. The fluorescence emission after the oxidation of Amplex Red reagent was measured by using Synergy H1 multimode plate reader (BioTek Instruments, Inc., Winooski, VT, USA). The excitation wavelength was fixed at 530 nm, and the fluorescence emission is measured at 590 nm. Dose v/s response inhibition curve was obtained using semi-log plot of concentration of inhibitors 1-3 versus fluorescence intensity emission to provide experimental IC50 values and compared with a known standard GCPII inhibitor, PMPA by following a similar procedure. The data analysis was performed using GraphPad Prism, version 6.00 for Windows (GraphPad Software, San Diego, CA).

### Example 21: Chemical Synthetic Strategy for the Preparation of Small Molecule Inhibitors

### Chemical Synthesis of Inhibitor Precursor 15a-j (FIG. 21):

A stirred solution of protected amino acids 11a-c (1 equiv.) and NaHCO₃ (1.2 equiv.) in CH₂Cl₂:H₂O (5v:0.5v or 1v:1v) was cooled to 0° C. α-Chloroacetyl chloride 12 (1.2 equiv.) was added dropwise over a period of 15 minutes at 0° C. to the reaction mixture. The reaction mixture was stirred for 1 h by allowing the temperature to raise to room temperature and further stirred for 1 h or 2 h. The reaction mixture was diluted with water and dichloromethane and the organic layer was separated and washed with sodium bicarbonate solution. The organic layer was dried over anhydrous sodium sulfate, filtered and concentrated under reduced pressure to afford 13a-c in 78-96% yields. Next, 13a-c were reacted with 14a-j in the presence of potassium iodide and K₂CO₃ in refluxing dry THF overnight to afford precursors 15a-j in 82-94% yields. The precursors 15a-j were completely characterized using various spectroscopic techniques and readily available for further experimental studies. In FIG. 21, R1 group of 15 a-j is from 13a-c and R2 group is from 14a-j.

These precursors were then used to prepare ligands 1-10.

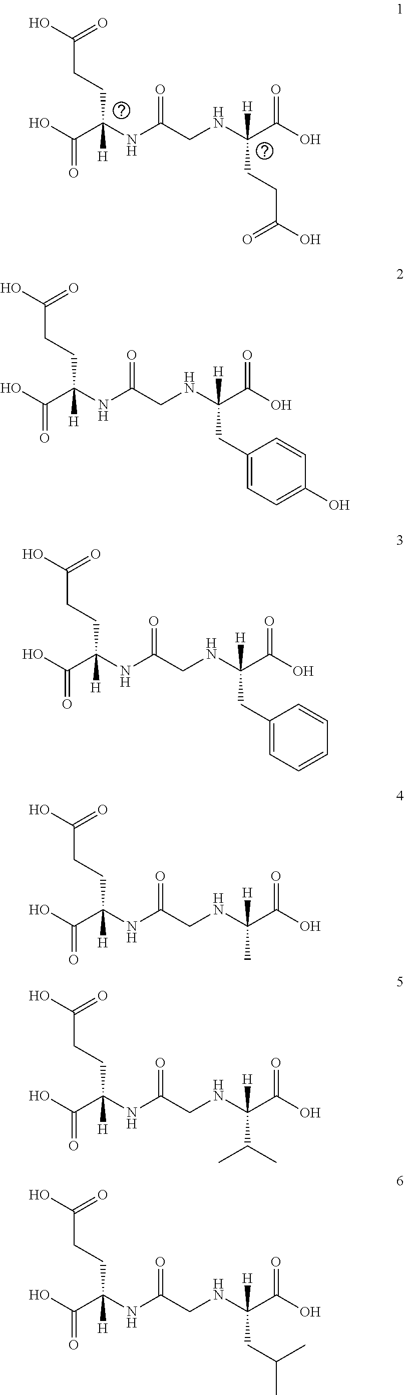

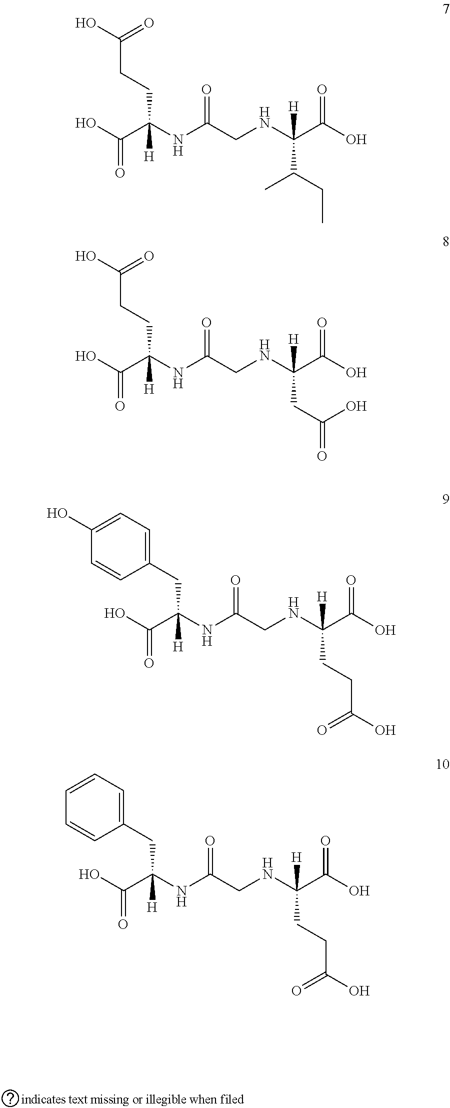

### Example 22: Evaluation of Binding Affinity of AAPT-PCa Bioconjugate 8a-g in PSMA⁺ LNCaP Cells

In an embodiment, FIG. 24, evaluation of binding affinity of AAPT-PCa Bioconjugate 8a in PSMA+ LNCaP cells was done, using gamma scintillation counter. The binding affinity constant of PSMA targeted conjugate, 8a, to target PSMA was determined in vitro in LNCaP cells by measuring the counts per minute for different concentrations of the ^{99m}Tc bioconjugate 8a in triplicate. A hyperbolic curve of different concentrations of ^{99m}Tc bioconjugates 8a against counts per minutes in the PSMA+ LNCaP cells yields a dissociation constant (KD) of 57 nM. The high dissociation constant value (57 nM) gives us undisputable evidence for the high affinity of 8a (atleast 6 times) as reported in Table 3 of the present invention of the prior art) in the radio ^{99m}Tc bioconjugate to PSMA protein and its perfect fit to the 20 Å protein tunnel present in PSMA.

Similarly, FIG. 25, FIG. 26, FIG. 27, FIG. 28, FIG. 29, FIG. 30 represents evaluation of binding affinity of AAPT-PCa bioconjugates 8b, 8c, 8d, 8e, 8f and 8g respectively in PSMA+ LNCaP cells. Bioconjugate 8b shows the binding affinity of 46 nM. bioconjugate 8c shows the binding affinity of 49 nM. bioconjugate 8d shows the binding affinity of 60 nM. bioconjugate 8e shows the binding affinity of 45 nM. bioconjugate 8f shows the binding affinity of 48 nM. bioconjugate 8g shows the binding affinity of 42 nM. xsPMPA has been used as control.

### Example 23: Procedure for Non-Radioactive Kit Preparation of Chelating Bioconjugates 8a-g

A solution of stannous chloride dihydrate (0.80 mg, 0.003 mmol) in 0.2 M hydrochloric acid (0.8 mL) was added to a solution of sodium-α-D-glucoheptonate dihydrate (800 mg, 2.815 mmol) in nitrogen-purged water. Chelating bioconjugate 8a-g (0.001 mmol) was added to the reaction mixture while continuous nitrogen purging. The pH of the solution was adjusted to 6.8 using 0.1 N sodium hydroxide solution. After the pH adjustment nitrogen-purged water was added to achieve a total volume of 10 mL. The solution was then dispensed in 5 mL vials (1 mL/vial) under nitrogen atmosphere and lyophilized for 48 h. The vials were then sealed under nitrogen atmosphere to yield the nonradioactive kits, which were stored at −20° C. until further use.

### Example 24: Procedure for Chelation of Radioisotope ^{99m}Tc with Bioconjugates 8a-g

In the Cold kit vial containing bioconjugate 8a-g, a solution of sodium pertechnate ^{99m}Tc (1 mL, 15 mCi) was added. The vial was kept for 18 min in boiling hot water rendering chelating bioconjugate, and it was cooled to room temperature before use.

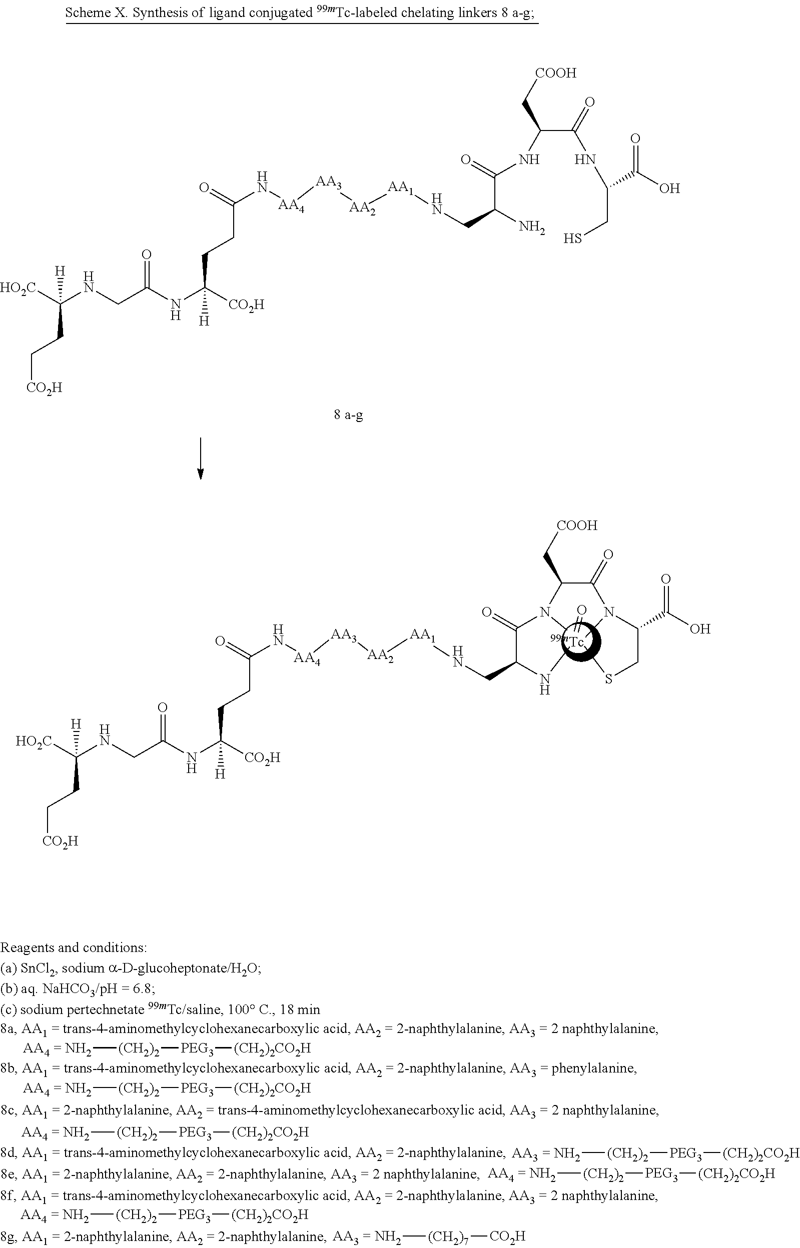

### Example 25: Procedure for Binding Affinity of Chelating Bioconjugates 8a-g

PSMA⁺ LNCaP cells were cultured in a T-75 flask for 48 h until reached 80%/confluency for binding affinity evaluation of bioconjugates 8a-g with PSMA protein. The cells were then gently dissociated with trypsin and centrifuge to obtain a pellet. Further, 1.2×10⁵′ cells/Eppendorf was added in 100 μL RPMI 1640 media and treated with varying concentrations of bioconjugates 8a-g (1215, 750, 300, 125, 100, 75, 50, 40, 30, 20, and 10 nM) in 400 μL of RPMI 1640 media and incubated in CO2 incubator for 1 h. For competitive experiments, cells were incubated with 2-PMPA (1215 nM×100-fold excess) for 1 h prior to treatment. After incubation, cells were pelleted down by centrifugation, and the spent medium was discarded. The treated cells were then washed twice with 500 μL of ice-cold FACS buffer (84 mg EDTA and 1 mL of 25 mM HEPES buffer in 50 mL 1× DPBS). The cell pellet free of unbound bioconjugate was resuspended in the ice-cold FACS buffer to evaluate cell-bound radioactivity using a gamma counter. The dissociation constant K_{d} was calculated using graph pad prism 7.04.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

All references, including granted patents and patent application publications, referred herein are incorporated herein by reference in their entirety.

1. A conjugate having a formula: D-E-F wherein
D comprises AAPT ligand or a derivative thereof and having formula I 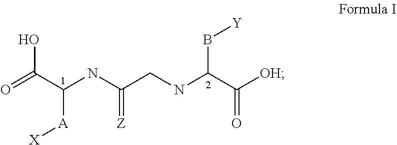
E is a spacer comprising a chain of amino acid or its derivatives comprising AA1, AA2, AA3 and/or AA4;
F is a chelating group comprises formula II 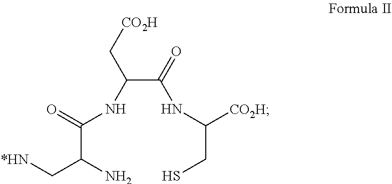
wherein A and B of formula I are independently selected from a group consisting of hydrogen, optionally substituted C₁-C₇ alkyl, and optionally substituted aryl groups; X and Y are selected from the groups comprising of —H, —OH, and —COOH groups, and Z is one of O or S groups;
wherein formula I comprises polar charged or polar uncharged amino acid or both;
wherein more than two of AA1, AA2, AA3 and/or AA4 are present;
wherein * indicates the site of attachment to the spacer;
wherein the conjugate is configured to have binding affinity≤60 nM.

2. The conjugate of claim 1, wherein formula I comprises polar negatively charged amino acid.

3. The conjugate of claim 1, wherein AA1 comprises trans-4-aminomethylcyclohexanecarboxylic acid or 2-naphthylalanine.

4. The conjugate of claim 1, wherein AA2 comprises 2-naphthylalanine or trans-4-aminomethylcyclohexanecarboxylic acid.

5. The conjugate of claim 1, wherein AA3 comprises 2-naphthylalanine or phenylalanine or NH₂—(CH₂)₂-PEG_{2/3}-(CH₂)₂CO₂H or NH₂—(CH₂)₇—CO₂H.

6. The conjugate of claim 1, wherein AA4 comprises NH₂—(CH₂)₂-PEG_{2/3}-(CH₂)₂CO₂H.

7. The conjugate of claim 1, wherein AA4 comprises Polyethylene Glycol 3 (PEG₃).

8. The conjugate of claim 1, comprising AAPT ligand conjugated arene chelating linker for delivery of ^{99m}Tc radioisotope comprises:

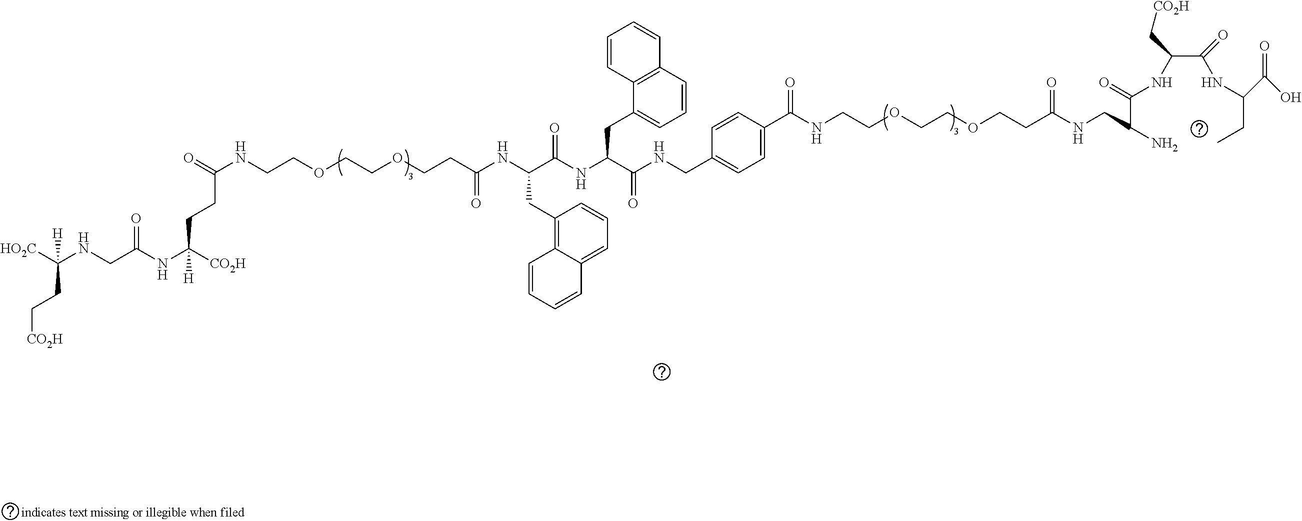

9. The conjugate of claim 1, wherein the conjugates having formula:

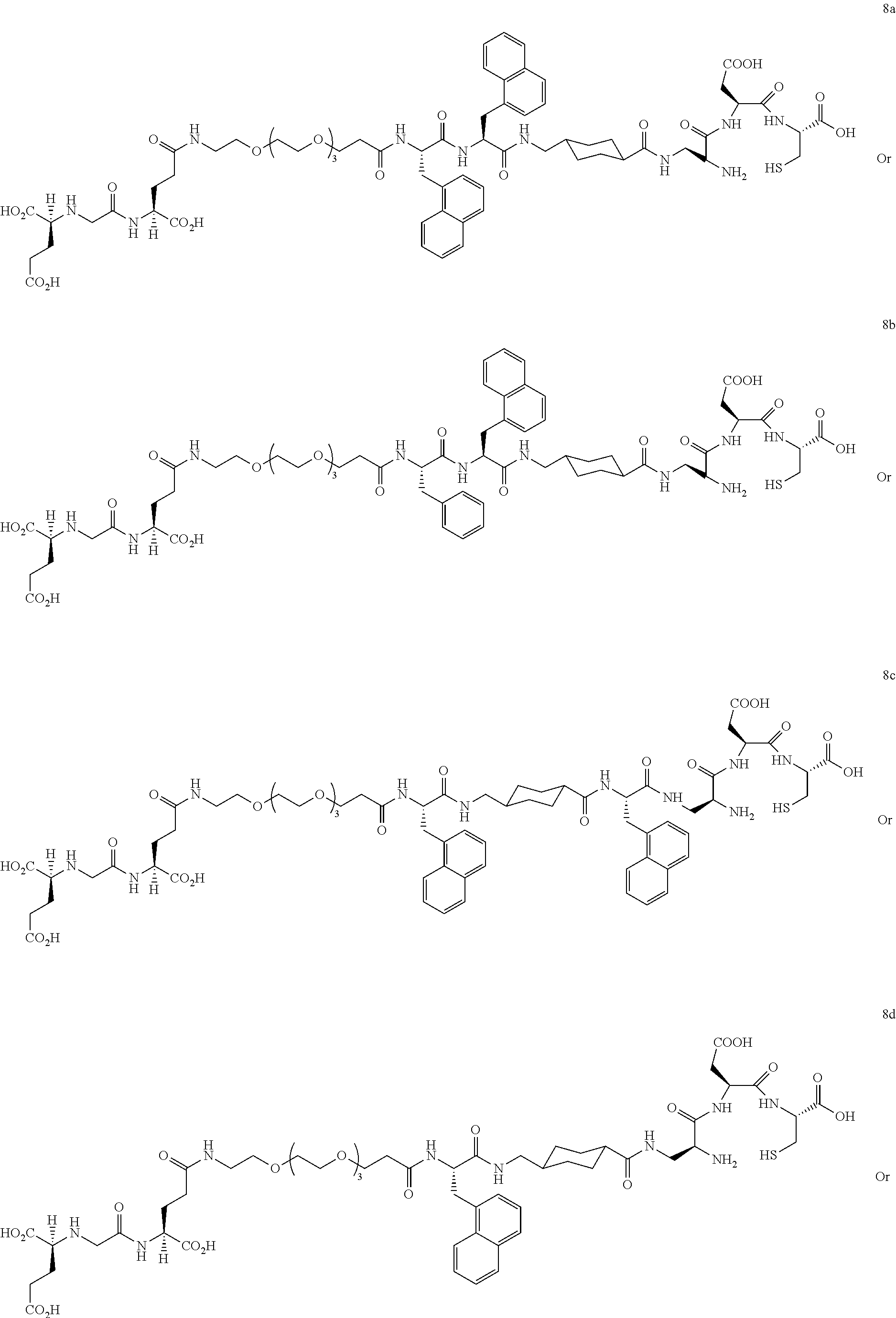

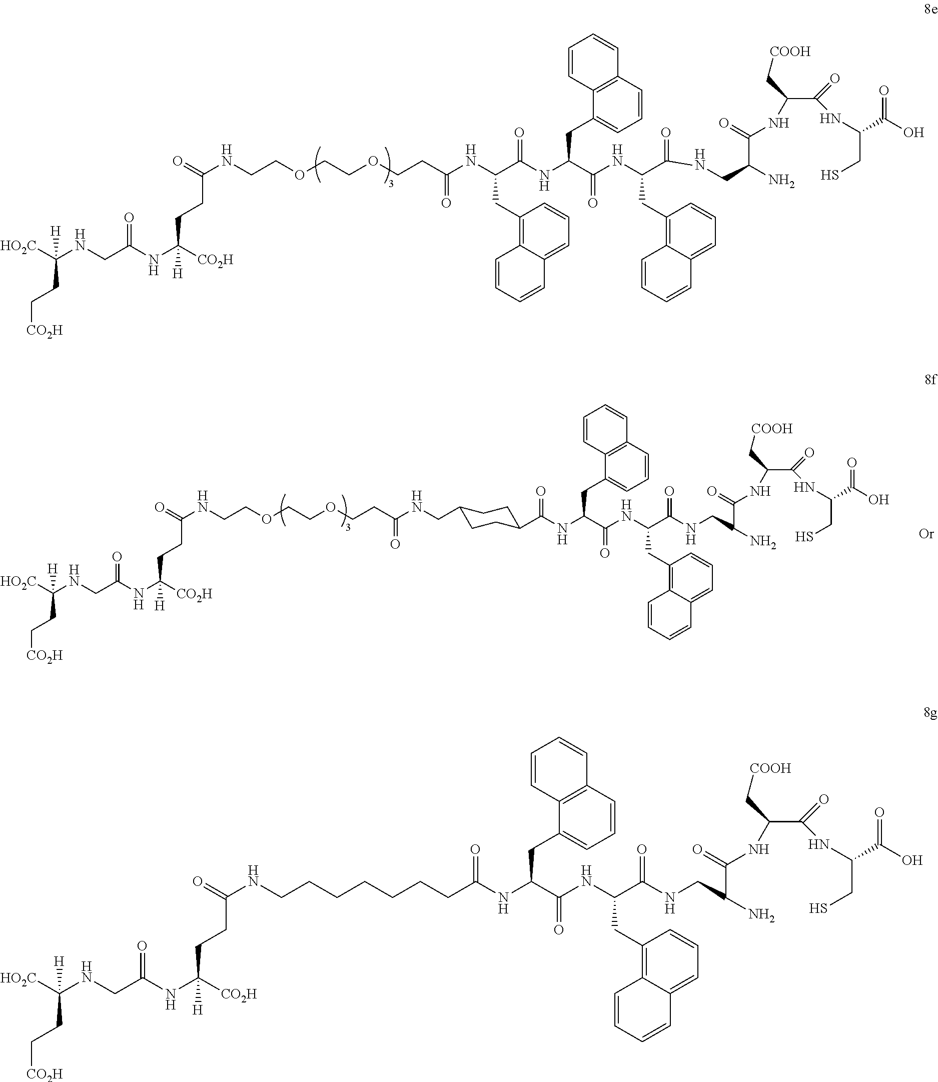

10. The conjugate of claim 1 is configured to be used for targeted drug delivery of radioisotopes for diagnosis and treatment of malignancy and neurodegenerative diseases where PSMA protein is overexpressed.

11. The conjugate of claim 1, wherein the amino acid residues of E are optionally substituted.

12. A method of preparation of precursor of AAPT ligand as following:
(a) stirring a solution of protected amino acids (1 equiv.) and NaHCO₃ (1.2 equiv.) in CH₂Cl₂:H₂O (5v:0.5v or 1v:1v) and cooling to 0° C. to get a reaction mixture;
(b) adding α-Chloroacetyl chloride (1.2 equiv.) dropwise over a period of 15 minutes at 0° C. to the reaction mixture of step (a) and stirring the reaction mixture for 1 h by allowing the temperature to raise to room temperature and further stirring for 1 h or 2 h;
(c) diluting the reaction mixture of step (b) with water and dichloromethane and separating an organic layer and washing it with sodium bicarbonate solution;
(d) drying the organic layer of step (c) over anhydrous sodium sulfate, filtering and concentrating under reduced pressure to get (S)-tert-Butyl 2-(2-chloroacetamido)-3-phenyl ester derivative in 78-96% yields;
(e) reacting (S)-tert-Butyl 2-(2-chloroacetamido)-3-phenyl ester derivative of step (d) with an amine derivative in the presence of potassium iodide and K₂CO₃ in refluxing dry THF overnight to get precursors of AAPT ligand in 82-94% yields;
(f) characterizing the precursors of AAPT ligand of using various spectroscopic techniques.

13. The method of preparation of precursor of AAPT ligand of claim 12, further comprises a method of solid phase synthesis of AAPT ligand conjugated with a spacer is as following:
(a) swelling of H-Cys-2-ClTrt resin (0.80-0.100 g) in a resin beads in CH₂Cl₂ (5-10 mL) for 30 minutes by bubbling nitrogen and after draining CH₂Cl₂, the resin is swelled once again with DMF (5 mL×3) thrice for 15 minutes each and then (i) adding Fmoc-Asp(O^{t}Bu)-OH (90-100 mg), PyBOP (110-120 mg) and DIPEA (0.1-0.2 mL) in DMF (0.5 mL) to the peptide vessel and continuing the coupling reaction for 6 h and then washing the resin with DMF thrice following by isopropanol thrice to perform a reaction; (ii) confirming the completion of the reaction by performing Kaiser test and then adding 20% Piperdine in DMF at room temperature and bubbling the resin beads for 30 min;
(b) adding Fmoc-diaminopropionic (DAP) acid, PyBOP and DIPEA in DMF for 6 h and then adding 20% Piperdine in DMF at room temperature and bubbling the resin beads for 30 min;
(c) adding Fmoc-AA1-OH, PyBOP and DIPEA in DMF for 6 h and then adding 20% Piperdine in DMF at room temperature and bubbling the resin beads for 30 min;
(d) adding Fmoc-AA₂-OH, PyBOP and DIPEA in DMF for 6 h and then adding 20% Piperdine in DMF at room temperature and bubbling the resin beads for 30 min;
(e) adding Fmoc-AA₃-OH, PyBOP and DIPEA in DMF for 6 h and then adding 20% Piperdine in DMF at room temperature and bubbling the resin beads for 30 min;
(f) adding Fmoc-AA₄-OH, PyBOP and DIPEA in DMF for 6 h and then adding 20% Piperdine in DMF at room temperature and bubbling the resin beads for 30 min;
(g) adding AAPT(O^{t}Bu)₃-OH, PyBOP, and DIPEA in DMF to the peptide vessel of step (f) for 6 h;
(h) adding 5 ml of a mixture of TFA to the resin beads and bubbling nitrogen gas through resin beads for 30 min and then 2.5 ml of the mixture of TFA for 15 min twice and collecting a mother liquor in a round bottom flask and then evaporating the mixture of TFA from the mother liquor to form concentrated viscous mixture;
(i) precipitating the AAPT ligand conjugated with the spacer from the concentrated viscous mixture of step (h) in ice cold diethylether.

14. The method of claim 13, wherein a mixture of TFA comprises TFA/H₂O/TIPS/EDT (92.5:2.5:2.5:2.5) or TFA/H₂O/TIPS (95:2.5:2.5).

15. A conjugate comprising:
a) a ligand;
b) a spacer; and
c) a chelating ligand;
wherein the ligand is a compound of Formula I 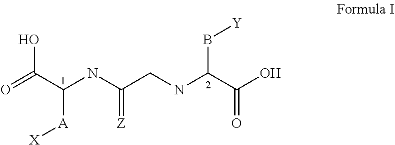
and stereoisomers thereof, wherein A and B are independently selected from a group consisting of hydrogen, optionally substituted C₁-C₇ alkyl, and optionally substituted aryl groups; X and Y are selected from the groups comprising of —H, —OH, and —COOH groups, and Z is one of O or S groups;
wherein the conjugate is a AAPT-PCa DOTA bioconjugate.

16. The conjugate of claim 15, wherein the spacer is a peptide comprising at least 2-20 amino acids.

17. The conjugate of claim 15, wherein the chelating ligand comprises a drug comprises at least one of imaging agents, anticancer drug or a radionuclide.

18. The conjugate of claim 15, wherein AAPT-PCa DOTA bioconjugate having formula:

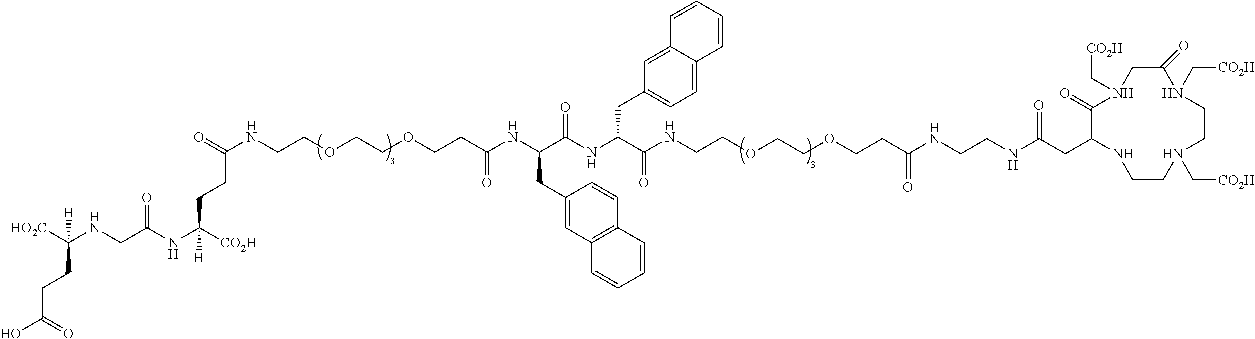

19. A method of solid phase synthesis of AAPT-PCa DOTA bioconjugate of claim 15, for delivering of radioisotopes comprising:
(a) swelling of Universal NovaTag resin in resin beads in a peptide vessel in 20% Piperidine in DMF at room temperature and bubbling the resin beads for 30 min and then adding Fmoc-NH—(PEG)₃-CO₂H, PyBOP and DIPEA in DMF to the peptide vessel and continuing the coupling reaction for 6 h and washing the resin with DMF thrice following by isopropanol thrice to perform a reaction;
(b) confirming the completion of the reaction by performing Kaiser test and then adding 20% Piperidine in DMF at room temperature and bubbling the resin beads for 30 min and adding Fmoc-2-Nal-OH twice, PyBOP and DIPEA in DMF for 6 h;
(c) confirming the completion of the reaction by performing Kaiser test and then adding 20% Piperidine in DMF at room temperature and bubbling the resin beads for 30 min and adding Fmoc-NH—(PEG)₃-COOH PyBOP and DIPEA in DMF for 6 h;
(d) adding 20% Piperidine in DMF at room temperature and bubbling the resin beads for 30 min and adding AAPT(O^{t}Bu)₃-OH, PyBOP, and DIPEA in DMF to the peptide vessel of step (c) for 6 h;
(e) adding 1M HOBt in DCM/TFE (1:1) for 10 min at room temperature and then adding DOTA-tris(tert-butyl ester), PyBOP and DIPEA in DMF for 6 h;
(f) adding 5 ml of a mixture of TFA to the resin beads and bubbling nitrogen gas through resin beads for 30 min and then 5 ml of the mixture of TFA for 5 min twice and collecting a mother liquor in a round bottom flask;
(g) evaporating the mixture of TFA from the mother liquor to form concentrated viscous mixture;
(h) precipitating the AAPT-PCa DOTA bioconjugate from the concentrated viscous mixture of step (g) in ice cold diethylether.

20. The method of claim 19 for delivering radioisotopes comprising ⁶⁴Cu, ⁶⁸Ga and ¹⁷⁷Lu as radioisotopes.
